(19) United States
(12) Patent Application Publication
(10) Pub. No.: US 2012/0208852 A1
(43) Pub. Date: Aug. 16, 2012

(54) Heterocyclic-substituted 2-acetamido-5-aryl-1,2,4-triazolones and use thereof

(75) Inventors: Chantal Fürstner, Mulheim/Ruhr (DE); Joerg Keldenich, Wuppertal (DE); Armin Kern, Wuppertal (DE); Martina Delbeck, Essen (DE); Peter Kolkhof, Wuppertal (DE); Axel Kretschmer, Wuppertal (DE); Elisabeth Pook, Wuppertal (DE); Carsten Schmeck, Mulheim (DE); Hubert Trübel, Wuppertal (DE)

(73) Assignee: BAYER PHARMA AKTIENGESELLSCHAFT, Berlin (DE)

(21) Appl. No.: 13/392,305

(22) PCT Filed: Aug. 24, 2010
(86) PCT No.: PCT/EP10/62354

§ 371 Date: May 3, 2012

(30) Foreign Application Priority Data
Aug. 27, 2009 (DE) 10 2009 028 929.1

(51) Int. Cl. A61K 31/4245; C07D 403/12; A61K 31/4196; A61P 9/00; A61P 1/16; A61P 7/00; A61P 5/10; C07D 401/12; A61K 31/4439; A61K 31/422; A61P 7/10; C07D 413/12; A61P 1/00

(52) U.S. Cl. 514/340; 548/132; 548/253; 548/131; 548/143; 514/364; 514/381; 548/235; 546/272.4; 548/263.2; 514/384; 514/374

(57) ABSTRACT

The present application relates to new, heterocyclyl-substituted 2-acetamido-5-aryl-1,2,4-triazolones, to processes for preparing them, to their use alone or in combinations for the treatment and/or prevention of diseases and also to their use for the production of medicaments for the treatment and/or prevention of diseases, more particularly for the treatment and/or prevention of cardiovascular disorders.

Heterocyclic-substituted 2-acetamido-5-aryl-1,2,4-triazolones and use thereof. The present application relates to new, heterocyclyl-substituted 2-acetamido-5-aryl-1,2,4-triazolones, to processes for preparing them, to their use alone or in combinations for the treatment and/or prevention of diseases and also to their use for the production of medicaments for the treatment and/or prevention of diseases, more particularly for the treatment and/or prevention of cardiovascular disorders.

The liquid content of the human body is subject to various physiological control mechanisms the purpose whereof is to keep it constant (volume homeostasis). In the process, both the volume filling of the vascular system and also the osmolarity of the plasma are continuously recorded by appropriate sensors (baroreceptors and osmoreceptors). The information which these sensors supply to the relevant centres in the brain regulate drinking behaviour and control fluid excretion via the kidneys by means of humoral and neural signals. The peptide hormone vasopressin is of central importance in this [Schrier R. W., Abraham, W. T., New Engl. J. Med. 341, 577-585 (1999)].

Vasopressin is produced in specialized endocrine neurones in the Nucleus supraopticus and N. paraventricularis in the wall of the third ventricle (hypothalamus) and transported from there along its neural processes into the posterior lobes of the hypophysis (neurohypophysis). There the hormone is released into the bloodstream according to stimulus. A loss of volume, e.g. as a result of acute bleeding, heavy sweating, prolonged thirst or diarrhoea, is a stimulus for intensified outpouring of the hormone. Conversely, the secretion of vasopressin is inhibited by an increase in the intravascular volume, e.g. as result of increased fluid intake.

Vasopressin exerts its action mainly via binding to three receptors, which are classified as V1a, V1b and V2 receptors and belong to the family of G protein-coupled receptors. V1a receptors are mainly located on the cells of the vascular smooth musculature. Their activation gives rise to vasoconstriction, as a result of which the peripheral resistance and blood pressure rise. Apart from this, V1a receptors are also detectable in the liver. V1b receptors (also named V3 receptors) are detectable in the central nervous system. Together with corticotropin-releasing hormone (CRH), vasopressin regulates the basal and stress-induced secretion of adrenocorticotropic hormone (ACTH) via the V1b receptor. V2 receptors are located in the distal tubular epithelium and the epithelium of the collecting tubules in the kidney. Their activation renders these epithelia permeable to water. This phenomenon is due to the incorporation of aquaporins (special water channels) in the luminal membrane of the epithelial cells.

The importance of vasopressin for the reabsorption of water from the urine in the kidney becomes clear from the clinical picture of diabetes insipidus, which is caused by a deficiency of the hormone, e.g. owing to hypophysis damage. Patients who suffer from this clinical picture excrete up to 20 litres of urine per 24 hours if they are not given replacement hormone. This volume corresponds to about 10% of the primary urine. Because of its great importance for the reabsorption of water from the urine, vasopressin is also synonymously referred to as antidiuretic hormone (ADH). Logically, pharmacological inhibition of the action of vasopressin/ADH on the V2 receptor results in increased urine excretion. In contrast to the action of other diuretics (thiazides and loop diuretics), however, V2 receptor antagonists cause increased water excretion, without substantially increasing the excretion of electrolytes. This means that by means of V2 antagonist drugs, volume homeostasis can be restored, without in the process affecting electrolyte homeostasis. Hence drugs with V2 antagonist activity appear particularly suitable for the treatment of all disease conditions which are associated with an overloading of the body with water, without the electrolytes being effectively increased in parallel. A significant electrolyte abnormality is measurable in clinical chemistry as hyponatraemia (sodium concentration <135 mmol/L); it is the most important electrolyte abnormality in hospital patients, with an incidence of ca. 5% or 250 000 cases per year in the USA alone. If the plasma sodium concentration falls below 115 mmol/L, comatose states and death are imminent.

Depending on the underlying cause, a distinction is made between hypovolaemic, euvolaemic and hypervolaemic hyponatraemia. The forms of hypervolaemia with oedema formation are clinically significant. Typical examples of this are syndrome of inappropriate ADH/vasopressin secretion (SIAD) (e.g. after craniocerebral trauma or as paraneoplasia in carcinomas) and hypervolaemic hyponatraemia in liver cirrhosis, various renal diseases and cardiac insufficiency [De Luca L. et al., Am. J. Cardiol. 96 (suppl.), 19L-23L (2005)]. In particular, patients with cardiac insufficiency, in spite of their relative hyponatraemia and hypervolaemia, often display elevated vasopressin levels, which is seen as the consequence of generally disturbed neurohumoral regulation in cardiac insufficiency [Francis G. S. et al., Circulation 82, 1724-1729 (1990)].

The disturbed neurohormonal regulation essentially manifests itself in an elevation of the sympathetic tone and inappropriate activation of the renin-angiotensin-aldosterone system. While the inhibition of these components by beta receptor blockers on the one hand and by ACE inhibitors or angiotensin receptor blockers on the other is now an inherent part of the pharmacological treatment of cardiac insufficiency, the inappropriate elevation of vasopressin secretion in advanced cardiac insufficiency is at present still not adequately treatable. Apart from the retention of water mediated by V2 receptors and the unfavourable haemodynamic consequences associated therewith in terms of increased backload, the emptying of the left ventricle, the pressure in the pulmonary blood vessels and cardiac output are also adversely affected by V1a-mediated vasoconstriction. Furthermore, on the basis of experimental data in animals, a direct hypertrophy-promoting action on the heart muscle is also attributed to vasopressin. In contrast to the renal effect of volume expansion, which is mediated by activation of V2 receptors, the direct action on the heart muscle is triggered by activation of V1a receptors.

For these reasons, substances which inhibit the action of vasopressin on the V2 and/or on the V1a receptor appear suitable for the treatment of cardiac insufficiency. In particular, compounds with combined activity on both vasopressin receptors (V1a and V2) should both have desirable renal and also haemodynamic effects and thus offer an especially ideal profile for the treatment of patients with cardiac insufficiency. The provision of such combined vasopressin antagonists also appears to make sense inasmuch as a volume diminution mediated solely via V2 receptor blockade can entail the stimulation of osmoreceptors and as a result a further compensatory increase in vasopressin release. As a result, in the absence of a component simultaneously blocking the V1a receptor, the harmful effects of the vasopressin, such as for example vasoconstriction and heart muscle hypertrophy, could be further intensified [Saghi P. et al., Europ. Heart J. 26, 538-543 (2005)].

WO 99/54315 discloses substituted triazolones with neuroprotective activity, and WO 2006/117657 describes triazolone derivatives as anti-inflammatory agents. Furthermore, EP 503 548-A1 and EP 587 134-A2 claim cyclic urea derivatives and their use for the treatment of thromboses. Substituted triazole thiones as ion channel modulators are disclosed in WO 2005/097112. WO 2007/134862 describes substituted imidazol-2-ones and 1,2,4-triazolones as vasopressin receptor antagonists for the treatment of cardiovascular disorders.

It is an object of the present invention to provide new compounds which act as potent, selective dual V1a/V2 receptor antagonists and are such as suitable for the treatment and/or prevention of diseases, more particularly for the treatment and/or prevention of cardiovascular disorders.

The present invention provides compounds of the general formula (I)

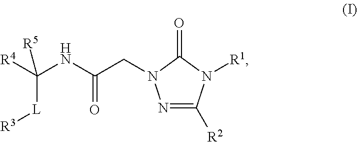

in which
L is a bond or —C(R^{6A}R^{6B})—*,
where
* is the attachment site to R³,
R^{6A} is hydrogen, (C₁-C₄) alkyl or trifluoromethyl,
R^{6B} is hydrogen or (C₁-C₄) alkyl,
   R¹ is (C₁-C₆) alkyl, (C₂-C₆) alkenyl, (C₂-C₆) alkynyl or (C₃-C₇) cycloalkyl,
where (C₁-C₆) alkyl, (C₂-C₆) alkenyl and (C₂-C₆) alkynyl may be substituted by 1 to 3 substituents independently of one another selected from the group consisting of halogen, cyano, oxo, hydroxyl, trifluoromethyl, (C₃-C₇) cycloalkyl, (C₁-C₆) alkoxy, trifluoromethoxy and phenyl,
   in which (C₃-C₇) cycloalkyl may be substituted by 1 or 2 substituents independently of one another selected from the group consisting of halogen, (C₁-C₄) alkyl, oxo, hydroxyl, (C₁-C₄) alkoxy and amino,
   and
   in which (C₁-C₆) alkoxy may be substituted by 1 or 2 substituents independently of one another selected from the group consisting of amino, hydroxyl, (C₁-C₄) alkoxy, hydroxycarbonyl and (C₁-C₄) alkoxycarbonyl,
   and
   in which phenyl may be substituted by 1 to 3 substituents independently of one another selected from the group consisting of halogen, cyano, nitro, (C₁-C₄) alkyl, trifluoromethyl, hydroxyl, hydroxymethyl, (C₁-C₄) alkoxy, trifluoromethoxy, (C₁-C₄) alkoxymethyl, hydroxycarbonyl, (C₁-C₄) alkoxycarbonyl, aminocarbonyl, mono-(C₁-C₄) alkylaminocarbonyl and di-(C₁-C₄) alkylaminocarbonyl,
and
where (C₃-C₇) cycloalkyl may be substituted by 1 or 2 substituents independently of one another selected from the group consisting of fluorine, (C₁-C₄) alkyl, (C₁-C₄) alkoxy, hydroxy, amino and oxo,
   R² is phenyl, thienyl or furyl,
where phenyl, thienyl and furyl may be substituted by 1 to 3 substituents independently of one another selected from the group consisting of halogen, cyano, nitro, (C₁-C₄) alkyl, trifluoromethyl, hydroxyl, (C₁-C₄) alkoxy and trifluoromethoxy,
   R³ is 5- or 6-membered heterocyclyl or 5- or 6-membered heteroaryl
where 5- or 6-membered heterocyclyl may be substituted by 1 to 3 substituents independently of one another selected from the group consisting of halogen, trifluoromethyl, (C₁-C₄) alkyl, hydroxyl, oxo, trifluoromethoxy, (C₁-C₄) alkoxy, amino, mono-(C₁-C₄)-alkylamino, di-(C₁-C₄)-alkylamino, (C₁-C₄) alkylthio and thiooxo,
and
where 5- or 6-membered heteroaryl may be substituted by 1 to 3 substituents independently of one another selected from the group consisting of halogen, trifluoromethyl, (C₁-C₄) alkyl, hydroxyl, trifluoromethoxy, (C₁-C₄) alkoxy, amino, mono-(C₁-C₄)-alkylamino, di-(C₁-C₄)-alkylamino and (C₁-C₄) alkylthio,
   R⁴ is phenyl, naphthyl or 5- to 10-membered heteroaryl,
where phenyl, naphthyl and 5- to 10-membered heteroaryl may be substituted by 1 to 3 substituents independently of one another selected from the group consisting of halogen, cyano, nitro, (C₁-C₄) alkyl, difluoromethyl, trifluoromethyl, hydroxyl, (C₁-C₄) alkoxy, difluoromethoxy and trifluoromethoxy,
   R⁵ is hydrogen, trifluoromethyl or (C₁-C₄) alkyl,
   and also their salts, solvates, and solvates of the salts.

Compounds according to the invention are the compounds of the formula (I) and their salts, solvates, and solvates of the salts; the compounds of the below-specified formulae embraced by formula (I), and their salts, solvates, and solvates of the salts; and also the compounds specified below as working examples and embraced by formula (I), and their salts, solvates, and solvates of the salts; in so far as the below-specified compounds embraced by formula (I) are not already salts, solvates, and solvates of the salts.

Depending on their structure, the compounds according to the invention may exist in stereoisomeric forms (enantiomers, diastereomers). The present invention therefore embraces the enantiomers or diastereomers and their respective mixtures. From such mixtures of enantiomers and/or diastereomers it is possible to isolate the stereoisomerically uniform constituents in a known way.

Where the compounds according to the invention are able to occur in tautomeric forms, the present invention embraces all of the tautomeric forms.

Salts preferred in the context of the present invention are physiologically unobjectionable salts of the compounds of the invention. Also embraced are salts which, while not themselves suitable for pharmaceutical applications, may nevertheless be used, for example, for the isolation or purification of the compounds of the invention.

Physiologically unobjectionable salts of the compounds of the invention embrace acid addition salts of mineral acids, carboxylic acids and sulphonic acids, examples being salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, maleic acid, citric acid, fumaric acid, maleic acid and benzoic acid.

Physiologically unobjectionable salts of the compounds of the invention also embrace salts with customary bases, such as—by way of example and preferably—alkali metal salts (e.g. sodium and potassium salts), alkaline earth metal salts (e.g. calcium and magnesium salts) and ammonium salts, derived from ammonia or from organic amines having 1 to 16 C atoms, such as—by way of example and preferably—ethylamine, diethylamine, triethylamine, ethyldiisopropylamine, monoethanolamine, diethanolamine, trisethanolamine, dicyclohexylamine, dimethylaminoethanol, procaine, dibenzylamine, N-methylmorpholine, arginine, lysine, ethylenediamine and N-methylpiperidine.

Solvates in the context of the invention are those forms of the compounds of the invention that form a complex in solid or liquid state by coordination with solvent molecules. Hydrates are one specific form of solvates, where the coordination is with water. Preferred solvates in the context of the present invention are hydrates.

Furthermore, the present invention also embraces prodrugs of the compounds of the invention. The term “prodrugs” embraces compounds which may themselves be biologically active or inactive but which during their residence time in the body are converted (metabolically or by hydrolysis, for example) into compounds of the invention.

In the context of the present invention, the substituents, unless otherwise specified, have the following definitions:

Alkyl in the context of the invention is a linear or branched alkyl radical having 1 to 6 or 1 to 4 carbon atoms. By way of example and for preference it includes the following: methyl, ethyl, n-propyl, isopropyl, n-butyl, isobutyl, 1-methylpropyl, tert-butyl, n-pentyl, isopentyl, 1-ethylpropyl, 1-methylbutyl, 2-methylbutyl, 3-methylbutyl, n-hexyl, 1-methylpentyl, 2-methylpentyl, 3-methylpentyl, 4-methylpentyl, 3,3-dimethylbutyl, 1-ethylbutyl and 2-ethylbutyl.

Cycloalkyl in the context of the invention is a monocyclic saturated alkyl radical having 3 to 7 or 3 to 6 carbon atoms. By way of example and for preference it includes the following: cyclopropyl, cyclobutyl, cyclopentyl, cyclohexyl and cycloheptyl.

Alkenyl in the context of the invention is a linear or a branched alkenyl radical having 2 to 6 carbon atoms and one or two double bonds. Preference is given to a straight-chain or branched alkenyl radical having 2 to 4 carbon atoms and one double bond. By way of example and for preference it includes the following: vinyl, allyl, isopropenyl and n-but-2-en-1-yl.

Alkynyl in the context of the invention is a linear or branched alkynyl radical having 2 to 6 carbon atoms and one triple bond. By way of example and for preference it includes the following: ethynyl, n-prop-1-yn-1-yl, n-prop-2-yn-1-yl, n-but-2-yn-1-yl and n-but-3-yn-1-yl.

Alkoxy in the context of the invention is a linear or branched alkoxy radical having 1 to 6 or 1 to 4 carbon atoms. By way of example and for preference it includes the following: methoxy, ethoxy, n-propoxy, isopropoxy, 1-methylpropoxy, n-butoxy, isobutoxy and tert-butoxy.

Alkoxycarbonyl in the context of the invention is a linear or branched alkoxy radical having 1 to 6 carbon atoms and a carbonyl group attached to the oxygen. By way of example and for preference it includes the following: methoxycarbonyl, ethoxycarbonyl, n-propoxycarbonyl, isopropoxycarbonyl and tert-butoxycarbonyl.

Mono-alkylaminocarbonyl in the context of the invention is an amino group which is linked via a carbonyl group and which has a linear or branched alkyl substituent having 1 to 4 carbon atoms. By way of example and for preference it includes the following: methylaminocarbonyl, ethylamino-carbonyl, n-propylaminocarbonyl, isopropylaminocarbonyl, n-butylaminocarbonyl and tert-butyl-aminocarbonyl.

Di-alkylaminocarbonyl in the context of the invention is an amino group which is linked via a carbonyl group and which has two identical or different linear or branched alkyl substituents each having 1 to 4 carbon atoms. By way of example and for preference it includes the following: N,N-dimethylaminocarbonyl, N,N-diethylaminocarbonyl, N-ethyl-N-methylaminocarbonyl, N-methyl-N-n-propylaminocarbonyl, N-n-butyl-N-methylamino carbonyl and N-tert-butyl-N-methylaminocarbonyl.

Heterocyclyl in the context of the invention is a saturated or partially unsaturated heterocycle having a total of 5 or 6 ring atoms, which comprises one to three ring heteroatoms from the series N, O and/or S, and is linked via a ring carbon atom or possibly a ring nitrogen atom. By way of example it includes the following: pyrrolidinyl, pyrazolidinyl, dihydropyrazolyl, imidazolidinyl, imidazolinyl, dihydrotriazolyl, tetrahydrofuranyl, oxazolidinyl, dihydroxazolyl, dihydrooxadiazolyl, dihydrothiazolidyl, piperidinyl, piperazinyl, tetrahydropyranyl, morpholinyl and thiomorpholinyl. Preference is given to a saturated or partially unsaturated heterocycle having a total of 5 ring atoms, which comprises one to three ring heteroatoms from the series N, O and/or S, and is linked via a ring carbon atom or possibly a ring nitrogen atom. By way of example and with preference it includes the following: imidazolinyl, oxazolidinyl, dihydrooxazolyl, dihydrotriazolyl, dihydrooxadiazolyl and dihydrothiazolidyl.

Heteroaryl in the context of the invention is a monocyclic or possibly bicyclic aromatic heterocycle (heteroaromatic) having a total of 5 to 10 ring atoms, which comprises up to three identical or different ring heteroatoms from the series N, O and/or S, and is linked via a ring carbon atom or possibly via a ring nitrogen atom. By way of example it includes the following: furyl, pyrrolyl, thienyl, pyrazolyl, imidazolyl, thiazolyl, oxazolyl, isoxazolyl, isothiazolyl, triazolyl, oxadiazolyl, thiadiazolyl, tetrazolyl, pyridyl, pyrimidinyl, pyridazinyl, pyrazinyl, triazinyl, benzofuranyl, benzothienyl, benzimidazolyl, benzoxazolyl, benzothiazolyl, benzotriazolyl, indolyl, indazolyl, quinolinyl, isoquinolinyl, naphthyridinyl, quinazolinyl, quinoxalinyl, phthalazinyl, pyrazolo[3,4-b]pyridinyl. Preference is given to monocyclic 5- or 6-membered heteroaryl radicals having up to three ring heteroatoms from the series N, O and/or S, such as, for example, furyl, thienyl, thiazolyl, oxazolyl, isothiazolyl, isoxazolyl, pyrazolyl, imidazolyl, triazolyl, oxadiazolyl, thiadiazolyl, tetrazolyl, pyridyl, pyrimidinyl, pyridazinyl, pyrazinyl, triazinyl.

Halogen in the context of the invention includes fluorine, chlorine, bromine and iodine. Preference is given to chlorine or fluorine.

An oxo group in the context of the invention is an oxygen atom attached via a double bond to a carbon atom.

A thiooxo group in the context of the invention is a sulphur atom attached via a double bond to a carbon atom.

If radicals in the compounds of the invention are substituted, the radicals, unless otherwise specified, may be substituted one or more times. In the context of the present invention it is the case that, for all radicals which occur more than once, their definitions are independent of one another. Substitution by one, two or three identical or different substituents is preferred. Very particular preference is given to substitution by one substituent.

Preference in the context of the present invention is given to compounds of the formula (I) in which
L is a bond or —C(R^{6A}R^{6B})—*,
   where
   * is the attachment site to R³,
   R^{6A} is hydrogen or methyl,
   R^{6B} is hydrogen or methyl,
   R¹ is (C₁-C₆) alkyl, (C₂-C₆) alkenyl or (C₃-C₆) cycloalkyl,
   where (C₁-C₆) alkyl and (C₂-C₆) alkenyl may be substituted by 1 to 3 substituents independently of one another selected from the group consisting of fluorine, chlorine, cyano, oxo, hydroxyl, trifluoromethyl, (C₃-C₆) cycloalkyl, (C₁-C₄) alkoxy, trifluoromethoxy and phenyl,
      in which (C₃-C₆) cycloalkyl may be substituted by 1 or 2 substituents independently of one another selected from the group consisting of fluorine, methyl, ethyl, oxo, hydroxyl, methoxy, ethoxy and amino,
      and
      in which phenyl may be substituted by a substituent selected from the group consisting of fluorine, chlorine, cyano, methyl, ethyl, trifluoromethyl, methoxy, ethoxy, trifluoromethoxy, methoxymethyl, ethoxymethyl, hydroxycarbonyl, methoxycarbonyl, ethoxycarbonyl and aminocarbonyl,
   and
   where (C₃-C₆) cycloalkyl may be substituted by 1 or 2 substituents independently of one another selected from the group consisting of fluorine, methyl, ethyl, methoxy, ethoxy, hydroxyl, amino and oxo,
R² is phenyl or thienyl,
   where phenyl and thienyl may be substituted by 1 or 2 substituents independently of one another selected from the group consisting of fluorine, chlorine, methyl, ethyl, trifluoromethyl, hydroxyl, methoxy, ethoxy and trifluoromethoxy,
R³ is 2-oxo-1,3-oxazolidin-5-yl, 2-oxo-1,3-oxazolidin-4-yl, 2-oxoimidazolidin-4-yl, 2-oxo-2,3-dihydro-1H-imidazol-4-yl, 4,5-dihydro-1H-imidazol-2-yl, 4,5-dihydro-1H-imidazol-4-yl, 4,5-dihydro-1H-imidazol-1-yl, 2-oxo-2,3-dihydro-1,3-oxazol-4-yl, 2-oxo-2,3-dihydro-1,3-oxazol-5-yl, 4,5-dihydro-1,3-oxazol-2-yl, 4,5-dihydro-1,3-oxazol-4-yl, 4,5-dihydro-1,3-oxazol-5-yl, 4,5-dihydro-5-oxo-1H-1,2,4-triazol-3-yl, 4,5-dihydro-5-oxo-1H-1,2,4-oxadiazol-3-yl, 4,5-dihydro-5-oxo-1,3,4-oxadiazol-2-yl, 4,5-dihydro-5-oxo-1H-1,2,4-thiadiazol-3-yl, 2,3-dihydro-2-oxo-1,3,4-thiadiazol-5-yl, furyl, thienyl, thiazolyl, oxazolyl, isothiazolyl, isoxazolyl, pyrazolyl, imidazolyl, triazolyl, oxadiazolyl, thiadiazolyl, tetrazolyl, pyridyl, pyrimidinyl, pyridazinyl, pyrazinyl or triazinyl,
   it being possible for 2-oxo-1,3-oxazolidin-5-yl, 2-oxo-1,3-oxazolidin-4-yl, 2-oxo-imidazolidin-4-yl, 2-oxo-2,3-dihydro-1H-imidazol-4-yl, 2-oxo-2,3-dihydro-1,3-oxazol-4-yl, 2-oxo-2,3-dihydro-1,3-oxazol-5-yl, 4,5-dihydro-5-oxo-1H-1,2,4-triazol-3-yl, 4,5-dihydro-5-oxo-1H-1,2,4-oxadiazol-3-yl, 4,5-dihydro-5-oxo-1,3,4-oxadiazol-2-yl, 4,5-dihydro-5-oxo-1H-1,2,4-thiadiazol-3-yl, 2,3-dihydro-2-oxo-1,3,4-thiadiazol-5-yl, to be substituted by 1 or 2 substituents independently of one another selected from the group consisting of trifluoromethyl, methyl and ethyl,
   and
   it being possible for 4,5-dihydro-1H-imidazol-2-yl, 4,5-dihydro-1H-imidazol-4-yl, 4,5-dihydro-1H-imidazol-1-yl, 4,5-dihydro-1,3-oxazol-2-yl, 4,5-dihydro-1,3-oxazol-4-yl, 4,5-dihydro-1,3-oxazol-5-yl to be substituted by 1 or 2 substituents independently of one another selected from the group consisting of oxo, methyl and ethyl,
   and
   it being possible for furyl, thienyl, thiazolyl, oxazolyl, isothiazolyl, isoxazolyl, pyrazolyl, imidazolyl, triazolyl, oxadiazolyl, thiadiazolyl, tetrazolyl, pyridyl, pyrimidinyl, pyridazinyl, pyrazinyl and triazinyl to be substituted by 1 or 2 substituents independently of one another selected from the group consisting of fluorine, chlorine, trifluoromethyl, methyl, ethyl, hydroxyl, trifluoromethoxy, methoxy, ethoxy, amino, methylamino, ethylamino, dimethylamino, methylethylamino and diethylamino,
R⁴ is phenyl,
   where phenyl may be substituted by 1 to 3 substituents independently of one another selected from the group consisting of fluorine, chlorine, cyano, methyl, ethyl, difluoromethyl, trifluoromethyl, hydroxyl, methoxy, ethoxy, difluoromethoxy and trifluoromethoxy,
R⁵ is hydrogen, methyl or ethyl,
   and also their salts, solvates, and solvates of the salts.

Particular preference is given in the context of the present invention to compounds of the formula (I) in which

L is a bond or —C(R^{6A}R^{6B})—*,
where
* is the attachment site to R³,
R^{6A} is hydrogen,
R^{6B} is hydrogen,
   R¹ is (C₂-C₄) alkyl, (C₂-C₄) alkenyl or cyclopropyl,
where (C₂-C₄) alkyl and (C₂-C₄) alkenyl are substituted by 1 or 2 substituents independently of one another selected from the group consisting of fluorine, hydroxyl, oxo and trifluoromethyl,
   R² is phenyl,
where phenyl is substituted by a substituent selected from the group consisting of fluorine and chlorine,
   R³ is a group of the formula

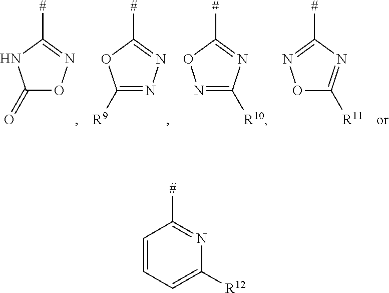

where
# is the attachment site to L,
R⁹ is hydrogen, trifluoromethyl, methyl or amino,
R¹⁰ is trifluoromethyl, methyl or amino,
R¹¹ is hydrogen, fluorine, trifluoromethyl or methyl,
R¹² is hydroxy or methoxy,
   R⁴ is a group of the formula

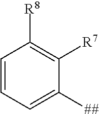

where
## is the attachment site to —C(R⁵)(LR³)N—,
R⁷ is hydrogen, fluorine, chlorine, trifluoromethyl and methoxy,
R⁸ is hydrogen, fluorine, chlorine, trifluoromethyl and methoxy,
where at least one of the radicals R⁷ and R⁸ is other than hydrogen,
   R⁵ is hydrogen or methyl,
   and also their salts, solvates, and solvates of the salts.

Preference is given in the context of the present invention as well to compounds of the formula (I) in which R² is p-chlorophenyl.

Preference is given in the context of the present invention as well to compounds of the formula (I) in which R¹ is 3,3,3-trifluoroprop-1-en-1-yl.

Preference is given in the context of the present invention as well to compounds of the formula (I) in which R¹ is 3,3,3-trifluoropropyl.

Preference is given in the context of the present invention as well to compounds of the formula (I) in which R¹ is 1,1,1-trifluoropropan-2-ol-3-yl.

Preference is given in the context of the present invention as well to compounds of the formula (I) in which

R¹ is (C₂-C₄) alkyl or (C₂-C₄) alkenyl,
where (C₂-C₄) alkyl and (C₂-C₄) alkenyl are substituted by 1 or 2 substituents independently of one another selected from the group consisting of fluorine, hydroxyl, oxo and trifluoromethyl.

Preference is given in the context of the present invention as well to compounds of the formula (I) in which R¹ is cyclopropyl.

Preference is given in the context of the present invention as well to compounds of the formula (I) in which R⁵ is hydrogen.

Preference is given in the context of the present invention as well to compounds of the formula (I) in which L is a bond.

Preference is given in the context of the present invention as well to compounds of the formula (I) in which

### L is —C(R^{6A}R^{6B})—*,

where
*is the attachment site to R³,
R^{6A} is hydrogen,
R^{6B} is hydrogen.

The radical definitions given individually in the respective combinations and preferred combinations of radicals are also replaced arbitrarily, independently of the particular radical combinations specified, by radical definitions from other combinations.

Very particular preference is given to combinations from two or more of the above-mentioned ranges of preference.

The invention further provides a process for preparing the compounds of the formula (I) according to the invention, characterized in that
[A] a compound of the formula (II)

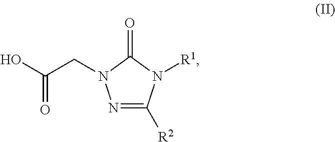

in which R¹ and R² are each as defined above
is coupled in an inert solvent, with activation of the carboxylic acid function, to a compound of the formula (III)

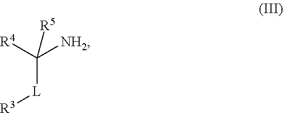

in which L, R³, R⁴ and R⁵ are each as defined above,
or
[B] a compound of the formula (IV)

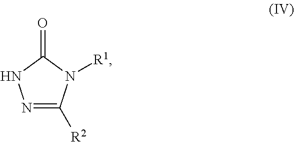

in which R¹ and R² are each as defined above
is reacted in an inert solvent, in the presence of a base, with a compound of the formula (V)

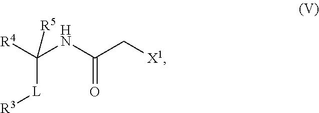

in which L, R³, R⁴ and
R⁵ are each as defined above
and
X¹ is a leaving group, such as halogen, mesylate or tosylate, for example,
or
[C] a compound of the formula (VI)

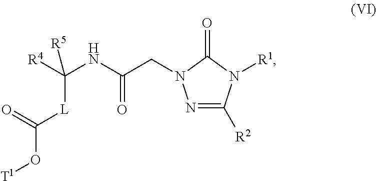

in which L, R¹, R², R⁴ and R⁵ are each as defined above,
and
T¹ is hydrogen or (C₁-C₄) alkyl,
is reacted in an inert solvent, optionally with activation of the carboxylic acid function with hydrazine, to give a compound of the formula (VII)

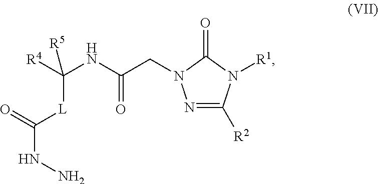

in which L, R¹, R², R⁴ and R⁵ are each as defined above,
which is subsequently cyclized in an inert solvent, optionally in the presence of a suitable base, with cyanogen bromide or a compound of the formula (VIII)

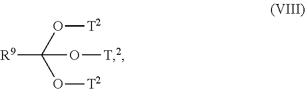

in which
R⁹ is (C₁-C₄) alkyl,
and
T² is (C₁-C₄) alkyl,
to give a compound of the formula (I-C1) or (I-C2)

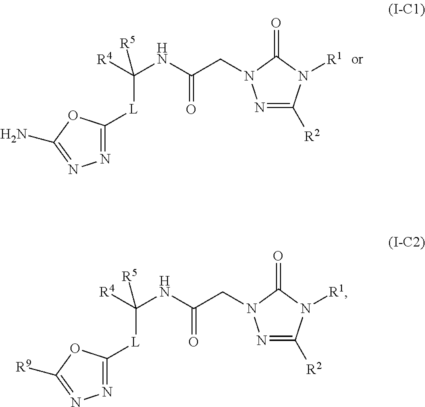

in which L, R¹, R², R⁴, R⁵ and R⁹ are each as defined above,
or
[D] a compound of the formula (VI) is reacted in an inert solvent, optionally with activation of the carboxylic acid function, with a compound of the formula (IX)

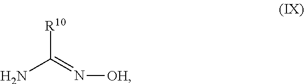

in which R¹⁹ is as defined above,
and the resulting intermediate is cyclized in a suitable solvent to give a compound of the formula (I-D)

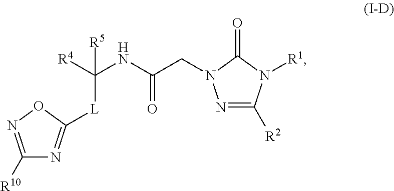

in which L, R¹, R², R⁴, R⁵ and R¹⁰ are each as defined above,
or
[E] a compound of the formula (X)

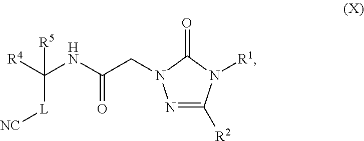

in which L, R¹, R², R⁴ and R⁵ are each as defined above,
is reacted in an inert solvent in the presence of a suitable base with hydroxylamine hydrochloride to give a compound of the formula (XI)

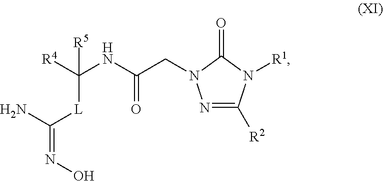

in which L, R¹, R², R⁴ and R⁵ are each as defined above, and this compound is subsequently cyclized in an inert solvent with a compound of the formula (XII-1) or (XII-2)

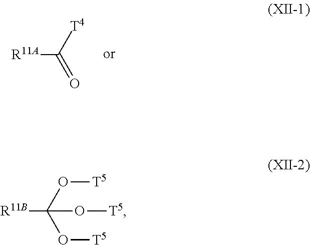

in which
R^{11A} is trifluoromethyl or (C₁-C₄) alkyl,
R^{11B} is hydrogen, trifluoromethyl or (C₁-C₄) alkyl,
T⁴ is chlorine, hydroxyl, (C₁-C₄) alkoxy, trifluoromethylcarbonyloxy or (C₁-C₄) alkylcarbonyloxy,
T⁵ is (C₁-C₄) alkyl,
to give a compound of the formula (I-E1) or (I-E2)

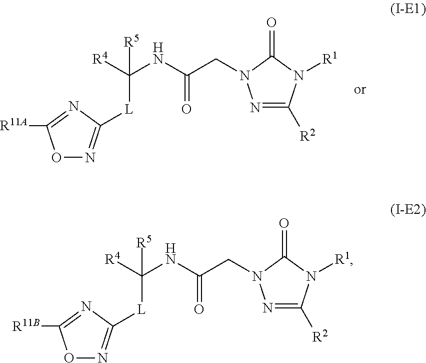

in which L, R¹, R², R⁴, R⁵, R^{11A} and a R^{11B} are each as defined above,
or
[F] a compound of the formula (X)

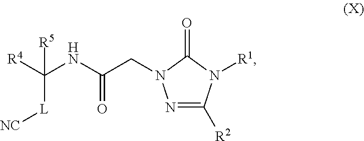

in which L, R¹, R², R⁴ and R⁵ are each as defined above,
is cyclized in an inert solvent in the presence of a suitable base with an azide reagent to give a compound of the formula (I-F)

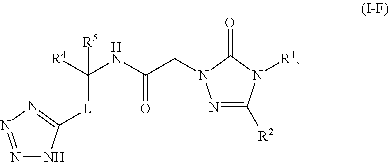

in which L, R¹, R², R⁴ and R⁵ are each as defined above,
or
[G] a compound of the formula (XI) is reacted in an inert solvent in the presence of a suitable base with phosgene, a phosgene derivative such as di- or triphosgene, N,N-carbonyldiimidazole or a chloroformic ester, and the resultant intermediate is cyclized directly further in an inert solvent, optionally in the presence of a suitable base, to give a compound of the formula (I-G)

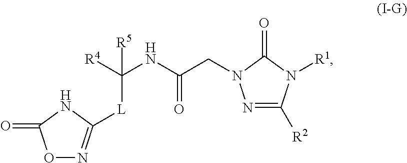

in which L, R¹, R², R⁴ and R⁵ are each as defined above,
   and the resulting compounds of the formula (I), (I-C1), (I-C2), (I-D), (I-E1), (IE2), (I-F) and (I-G) are converted optionally with the corresponding (i) solvents and/or (ii) bases or acids into their solvates, salts and/or solvates of the salts.

Inert solvents for the process steps (II)+(III)→(I), (VI)→(VII) and (VI)+(IX)→(I-D) are for example ethers such as diethyl ether, dioxan, tetrahydrofuran, glycol dimethyl ether or diethylene-glycol dimethyl ether, hydrocarbons such as benzene, toluene, xylene, hexane, cyclohexane or petroleum fractions, halogenated hydrocarbons such as dichloromethane, trichloromethane, tetra-chloromethane, 1,2-dichloroethane, trichloroethylene or chlorobenzene, or other solvents such as acetone, ethyl acetate, acetonitrile, pyridine, dimethyl sulphoxide, N,N-dimethylformamide, N,N′-dimethylpropyleneurea (DMPU) or N-methylpyrrolidone (NMP). Likewise it is possible to use mixtures of the said solvents. Dichloro-methane, tetrahydrofuran, dimethylformamide or mixtures of these solvents are preferred.

Suitable condensation agents for the amidation in the process steps (II)+(III)→(I), (VI)→(VII) and (VI)+(IX)→(I-D) include, for example, carbodiimides such as N,N′-diethyl-, N,N′-dipropyl-, N,N′-diisopropyl- or N,N′-dicyclohexylcarbodiimide (DCC) or N-(3-dimethylaminoisopropyl)-N′-ethylcarbodiimide hydrochloride (EDC), phosgene derivatives such as N,N′-carbonyldiimidazole (CDI), 1,2-oxazolium compounds such as 2-ethyl-5-phenyl-1,2-oxazolium-3 sulphate or 2-tert-butyl-5-methyl-isoxazolium perchlorate, acylamino compounds such as 2-ethoxy-1-ethoxy-carbonyl-1,2-dihydroquinoline, or isobutyl chloroformate, propanephosphonic anhydride, diethyl cyanophosphonate, bis-(2-oxo-3-oxazolidinyl)phosphoryl chloride, benzotriazol-1-yloxy-tris(dimethylamino)phosphonium hexafluorophosphate, benzotriazol-1-yloxy-tris(pyrrolidino)-phosphonium hexafluorophosphate (PyBOP), O-(benzotriazol-1-yl)-N,N,N′,N′-tetramethyluronium tetrafluoroborate (TBTU), O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU), O-(7-azabenzotriazol-1-yl)-N,N,N′,N′-tetramethyluronium hexafluorophosphate (HATU) or O-(1H-6-chlorobenzotriazol-1-yl)-1,1,3,3-tetramethyluronium tetrafluoroborate (TCTU), optionally in combination with other additives such as 1-hydroxy-benzotriazole (HOBt) or N-hydroxysuccinimide (HOSu), and, as bases, alkali metal carbonates, e.g. sodium or potassium carbonate or hydrogen carbonate, or organic bases such as trialkylamines, e.g. triethylamine, N-methylmorpholine, N-methylpiperidine or N,N-diisopropylethylamine.

Preferably EDC in combination with HOBt or TBTU in combination with N,N-diiso-propylethylamine is used.

The process steps (II)+(III)→(I), (VI)→(VII) and (VI)+(IX)→(I-D) are generally performed in a temperature range from −20° C. to +60° C., preferably at 0° C. to +40° C. The reaction can take place under standard atmospheric, increased or reduced pressure (e.g. from 0.5 to 5 bar). The operation is generally carried out under atmospheric pressure.

Inert solvents for the process step (IV)+(V)→(I) are for example halogenated hydrocarbons such as dichloromethane, trichloromethane, tetrachloromethane, trichloroethylene or chlorobenzene, ethers such as diethyl ether, dioxane, tetrahydrofuran, glycol dimethyl ether or diethylene glycol dimethyl ether, hydrocarbons such as benzene, toluene, xylene, hexane, cyclohexane or petroleum fractions, or other solvents such as acetone, methyl ethyl ketone, ethyl acetate, acetonitrile, N,N-dimethylformamide, dimethyl sulphoxide, N,N′-dimethylpropyleneurea (DMPU), N-methyl-pyrrolidone (NMP) or pyridine. Likewise it is possible to use mixtures of the said solvents. Preferably, acetonitrile, acetone or dimethylformamide is used.

As bases for the process step (IV)+(V)→(I), the usual inorganic or organic bases are suitable. These preferably include alkali metal hydroxides such as for example lithium, sodium or potassium hydroxide, alkali metal or alkaline earth metal carbonates such as lithium, sodium, potassium, calcium or caesium carbonate, alkali metal alcoholates such as sodium or potassium methanolate, sodium or potassium ethanolate or sodium or potassium tert-butylate, alkali metal hydrides such as sodium or potassium hydride, amides such as sodamide, lithium or potassium bis(trimethylsilyl)-amide or lithium diisopropylamide, or organic amines such as triethylamine, N-methylmorpholine, N-methylpiperidine, N,N-diisopropylethylamine, 1,5-diazabicyclo-[4.3.0]non-5-ene (DBN), 1,8-diazabicyclo[5.4.0]undec-7-ene (DBU) or 1,4-diazabicyclo[2.2.2]octane (DABCO®). Preferably, potassium carbonate or caesium carbonate is used.

In this step, the base is used in an amount of 1 to 5 mol, preferably in an amount of 1 to 2.5 mol, based on 1 mol of the compound of the formula (IV). The reaction generally takes place in a temperature range from 0° C. to +100° C., preferably at +20° C. to +80° C. The reaction can take place under standard atmospheric, increased or reduced pressure (e.g. from 0.5 to 5 bar). The operation is generally carried out under atmospheric pressure.

Inert solvents for the process step (VII)→(I-C1) are, for example, alcohols such as methanol, ethanol, n-propanol, isopropanol, n-butanol or tert-butanol, ethers such as diethyl ether, dioxane, tetrahydrofuran, glycol dimethyl ether or diethylene glycol dimethyl ether, hydrocarbons such as benzene, xylene, toluene, hexane, cyclohexane or petroleum fractions, or other solvents such as dimethylformamide (DMF), dimethyl sulphoxide (DMSO), N,N′-dimethylpropyleneurea (DMPU), N-methylpyrrolidone (NMP), pyridine, acetonitrile or else water. It is also possible to use mixtures of the stated solvents. Preference is given to using methanol.

Suitable bases for the process step (VII)→(1-C1) are the usual inorganic bases. These include alkali metal or alkaline earth metal carbonates such as lithium, sodium, potassium, calcium or caesium carbonate and alkali metal hydrogen carbonates such as sodium or potassium hydrogen carbonate.

The process step (VII)→(1-C1) is generally performed in a temperature range from 0° C. to +80° C., preferably at +20° C. to +60° C. The reaction can take place under standard atmospheric, increased or reduced pressure (e.g. from 0.5 to 5 bar). The operation is generally carried out under atmospheric pressure.

Inert solvents for the process step (XI)→(I-G) are for example halogenated hydrocarbons such as dichloromethane, trichloromethane, tetrachloromethane, trichloroethylene or chlorobenzene, ethers such as diethyl ether, dioxane, tetrahydrofuran, glycol dimethyl ether or diethylene glycol dimethyl ether, hydrocarbons such as benzene, toluene, xylene, hexane, cyclohexane or petroleum fractions, or other solvents such as acetone, methyl ethyl ketone, ethyl acetate, acetonitrile, N,N-dimethylformamide, dimethyl sulphoxide, N,N′-dimethylpropyleneurea (DMPU), N-methyl-pyrrolidone (NMP) or pyridine. Likewise it is possible to use mixtures of the said solvents. Preferably, DMSO or DMF is used.

Inert solvents for the process step (X)→(XI) are, for example, alcohols such as methanol, ethanol, n-propanol, isopropanol, n-butanol or tert-butanol, hydrocarbons such as benzene, toluene, xylene, hexane, cyclohexane or petroleum fractions, or water. It is also possible to use mixtures of the stated solvents. Preferably, methanol, ethanol, toluene or water is used.

As bases for the process steps (X)+(XI) and (XI)→(I-G), the usual inorganic or organic bases are suitable. These preferably include alkali metal hydroxides such as for example lithium, sodium or potassium hydroxide, alkali metal or alkaline earth metal carbonates such as lithium, sodium, potassium, calcium or caesium carbonate, alkali metal alcoholates such as sodium or potassium methanolate, sodium or potassium ethanolate or sodium or potassium tert-butylate, alkali metal hydrides such as sodium or potassium hydride, amides such as sodamide, lithium or potassium bis(trimethylsilyl)amide or lithium diisopropylamide, or organic amines such as triethylamine, N-methylmorpholine, N-methylpiperidine, N,N-diisopropylethylamine, pyridine, 1,5-diazabicyclo-[4.3.0]non-5-ene (DBN), 1,8-diazabicyclo[5.4.0]undec-7-ene (DBU) or 1,4-diazabicyclo[2.2.2]-octane (DABCO®). Preferably, triethylamine or pyridine or potassium tert-butylate is used.

The process step (XI)→(I-G) is carried out with particular preference in DMF in the presence of potassium tert-butylate.

The process step (X)→(XI) is carried out generally in a temperature range from +30° C. to +100° C., preferably at +50° C. to +80° C. The reaction can take place under standard atmospheric, increased or reduced pressure (e.g. from 0.5 to 5 bar). The operation is generally carried out under atmospheric pressure.

The process step (XI)→(I-G) is carried out generally in a temperature range from −10° C. to +50° C., preferably at 0° C. to +30° C. The reaction can take place under standard atmospheric, increased or reduced pressure (e.g. from 0.5 to 5 bar). The operation is generally carried out under atmospheric pressure.

The process step (XI)+(XII-1)→(I-E1) is carried out in the case of reaction of acids in accordance with the coupling conditions stated for the process step (II)+(III)→(I).

Suitable inert solvents for the process step (XI)+(XII-1)→(I-E1), when carboxylic anhydrides are reacted, include ethers such as diethyl ether, dioxane, tetrahydrofuran, glycol dimethyl ether or diethylene glycol dimethyl ether, hydrocarbons such as benzene, toluene, xylene, hexane, cyclohexane or petroleum fractions, halogenated hydrocarbons such as dichloromethane, trichloromethane, tetrachloromethane, 1,2-dichloroethane, trichloroethylene or chlorobenzene, or other solvents such as acetone, ethyl acetate, acetonitrile, pyridine, dimethyl sulphoxide, N,N-dimethylformamide, N,N′-dimethylpropyleneurea (DMPU) or N-methylpyrrolidone (NMP).

The reaction of carboxylic anhydrides in the process step (XI)+(XII-1)→(I-E1) takes place in the presence of a suitable base such as, for example, organic amines such as triethylamine, N-methyl-morpholine, N-methylpiperidine, N,N-diisopropylethylamine, pyridine, 1,5-diazabicyclo[4.3.0]non-5-ene (DBN), 1,8-diazabicyclo[5.4.0]undec-7-ene (DBU) or 1,4-diazabicyclo[2.2.2]octane (DABCO®). Preferably, triethylamine is used.

The process step (XI)+(XII-1)→(I-E1) with carboxylic anhydrides is carried out generally in a temperature range from +20° C. to +120° C., preferably at +50° C. to +80° C. The reaction can take place under standard atmospheric, increased or reduced pressure (e.g. from 0.5 to 5 bar). The operation is generally carried out under atmospheric pressure.

The process steps (VII)+(VIII)→(I-C2) and (XI)+(XII-2)→(I-E2) can be carried out with and without solvent. Examples of suitable inert solvents in this context include ethers such as diethyl ether, dioxane, tetrahydrofuran, glycol dimethyl ether or diethylene glycol dimethyl ether, hydrocarbons such as benzene, toluene, xylene, hexane, cyclohexane or petroleum fractions, halogenated hydrocarbons such as dichloromethane, trichloromethane, tetrachloromethane, 1,2-dichloroethane, trichloroethylene or chlorobenzene, or other solvents such as acetone, ethyl acetate, acetonitrile, pyridine, dimethyl sulphoxide, N,N-dimethylformamide, N,N′-dimethylpropyleneurea (DMPU) or N-methylpyrrolidone (NMP).

Examples of suitable Lewis acids for the process steps (VII)+(VIII)→(I-C2) and (XI)+(XII-2)→(I-E2) include boron trifluoride-diethyl ether complex, cerium(IV) ammonium nitrate (CAN), tin(II) chloride, lithium perchlorate, zinc(II) chloride, indium(III) chloride or indium(III) bromide. Preferably, boron trifluoride-diethyl ether complex is used. In this reaction, the Lewis acid may be used in an amount of 0.2 to 2.0 mol, preferably of 0.7 to 1.2 mol, based on 1 mol of the compound of the formula (II).

Process steps (VII)+(VIII)→(I-C2) and (XI)+(XII-2)→(I-E2) are carried out generally in a temperature range from +20° C. to +120° C., preferably at +50° C. to +80° C. The reactions can take place under standard atmospheric, increased or reduced pressure (e.g. from 0.5 to 5 bar). The operation is generally carried out under atmospheric pressure.

Examples of inert solvents for the reaction (XI)→(I-G) are ethers such as diethyl ether, dioxane, tetrahydrofuran, glycol dimethyl ether or diethylene glycol dimethyl ether, hydrocarbons such as benzene, toluene, xylene, hexane, cyclohexane or petroleum fractions, or other solvents such as dimethyl sulphoxide, dimethylformamide, N,N′-dimethylpropyleneurea (DMPU) or N-methyl-pyrrolidone (NMP). It is also possible to use mixtures of the stated solvents. Preferably, toluene or DMF is used.

A particularly suitable azide reagent in the process step (X)→(I-F) is sodium azide in the presence of ammonium chloride or trimethylsilyl azide. The latter reaction may be carried out advantageously in the presence of a catalyst. Particularly suitable for this purpose are compounds such as di-n-butyltin oxide, trimethylaluminium or zinc bromide. It is preferred to use trimethylsilyl azide in combination with di-n-butyltin oxide.

The reaction (X)→(I-F) is carried out in general in a temperature range from +50° C. to +150° C., preferably at +60° C. to +110° C. The reaction can be carried out under standard atmospheric, increased or reduced pressure (e.g. from 0.5 to 5 bar). The operation is generally carried out under atmospheric pressure.

The preparation of the compounds of the invention can be illustrated by the following synthesis schemes:

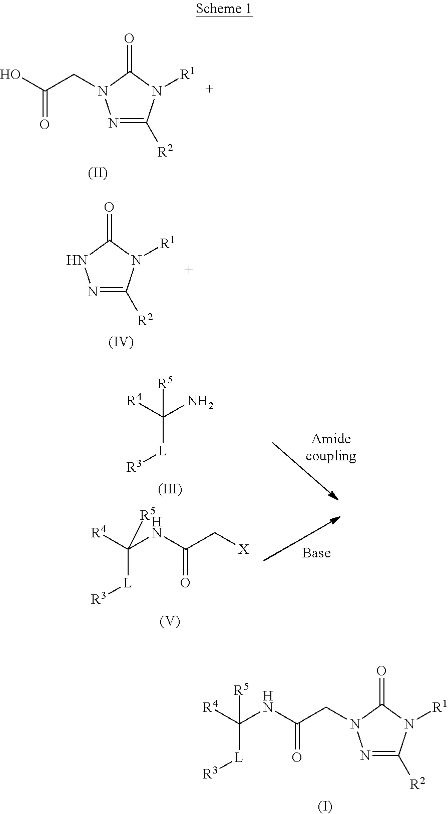

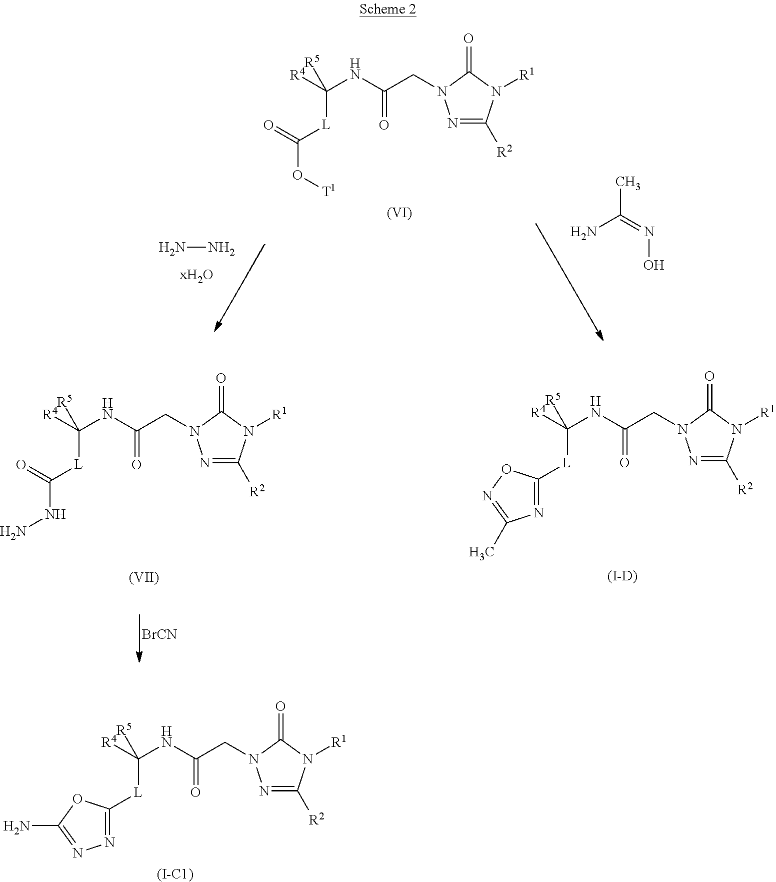

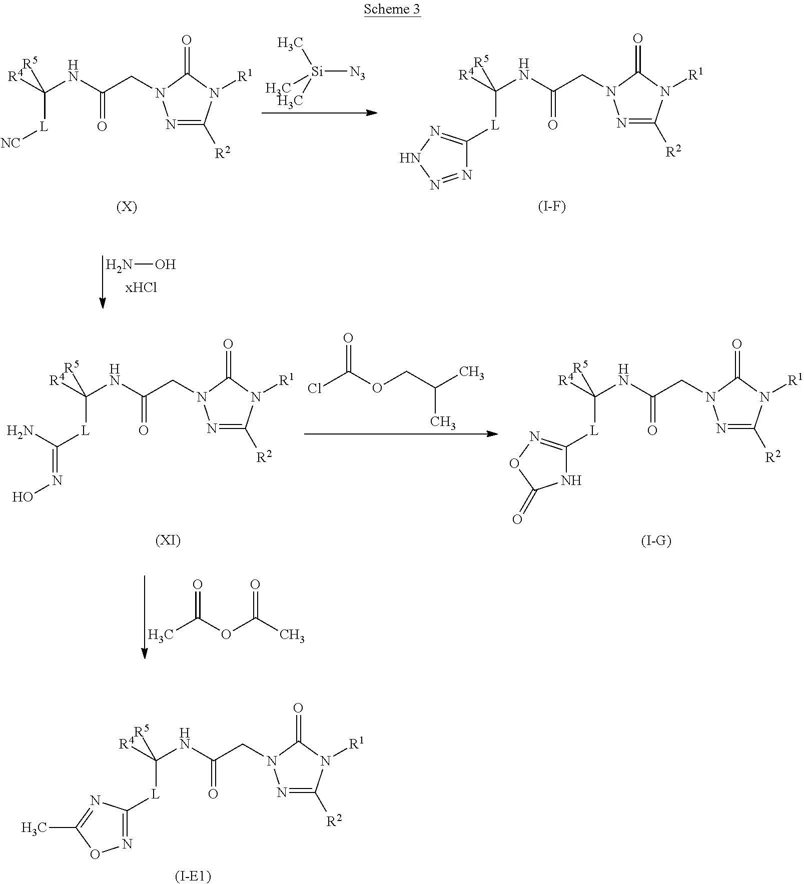

The compounds of the formula (II) can be obtained by base-induced alkylation of 5-aryl-2,4-dihydro-3H-1,2,4-triazol-3-ones to give the N²-substituted compounds and subsequent ester hydrolysis (see Scheme 4):

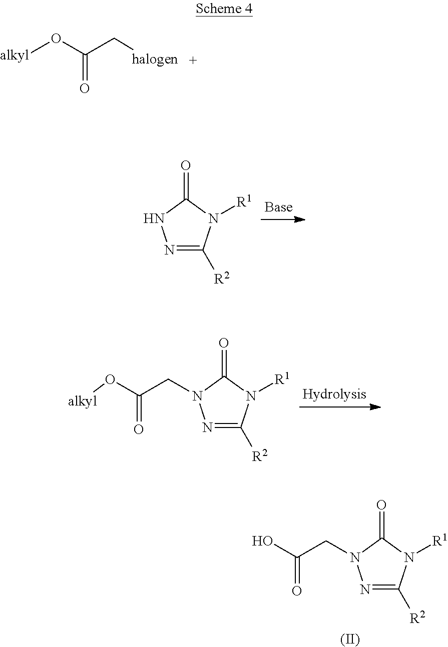

The N²-substituted compounds may alternatively also be prepared from N-(alkoxycarbonyl)arylthioamides, which are known from the literature [see, for example, M. Arnswald, W. P. Neumann, J. Org. Chem. 58 (25), 7022-7028 (1993); E. P. Papadopoulos, J. Org. Chem. 41 (6), 962-965 (1976)], by reaction with hydrazino esters and subsequent alkylation at N-4 of the triazolone (Scheme 5):

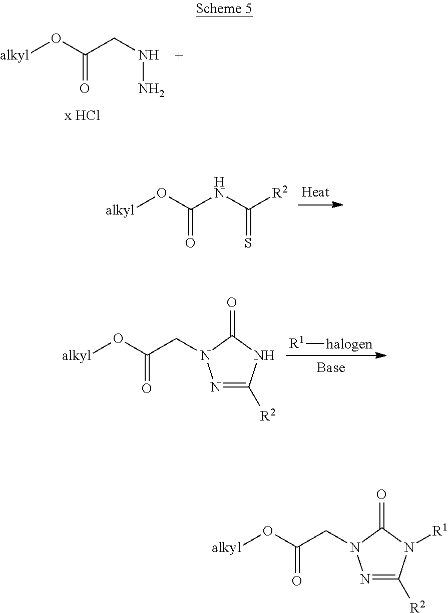

The compounds of the formula (IV) may be prepared starting from carboxylic hydrazides by reaction with isocyanates or nitrophenyl carbamates and subsequent base-induced cyclisation of the intermediate hydrazinecarboxamides (Scheme 6):

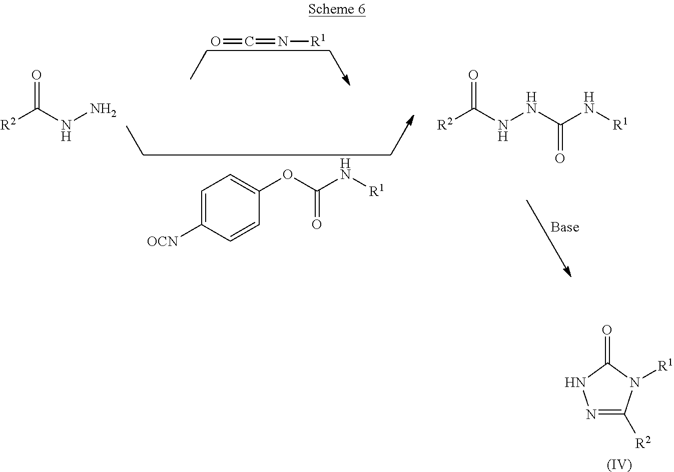

The compounds of the formula (III) are available commercially, known from the literature or may be prepared as shown by way of example in synthesis Schemes 7 and 8 below:

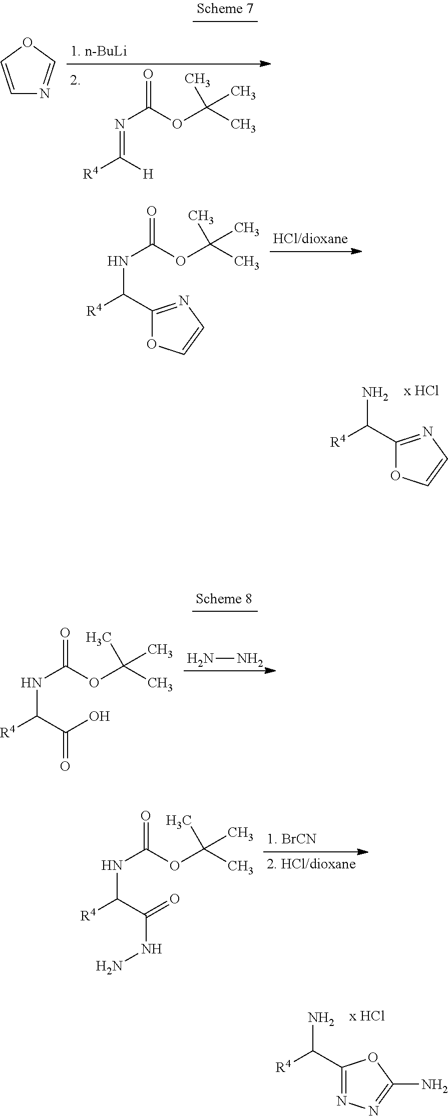

The compounds of the formulae (VI) and (X) can be prepared in analogy to process [A] and to WO 2007/134862.

The compounds of the formula (X) can alternatively also be prepared by reacting a carboxylic acid of the formula (VI-1) to give the corresponding amide, with subsequent dehydration (see Scheme 9).

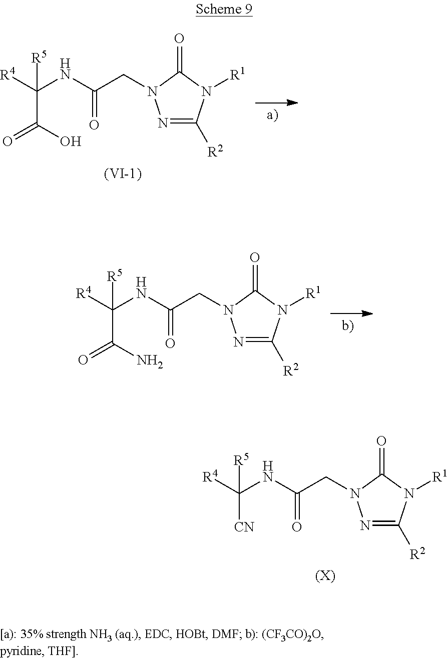

The compounds of the formulae (V), (VIII), (IX), (XII-1) and (XII-2) are variously available commercially, known from the literature, or can be prepared in analogy to processes known from the literature (for (V) cf., for example, WO 2007/134862), or as described in the present experimental section.

Further compounds of the invention may also be prepared, if desired, by conversions of functional groups of individual substituents, particularly those listed under R¹ and R³, starting from the compounds of the formula (I) obtained in accordance with processes above. These conversions are carried out in accordance with customary methods known to a person skilled in the art, and include, for example, reactions such as nucleophilic and electrophilic substitutions, oxidations, reductions, hydrogenations, transition metal-catalysed coupling reactions, eliminations, alkylation, amination, esterification, ester cleavage, etherification, ether cleavage, especially formation of carboxamides, and also introduction and removal of temporary protecting groups.

The compounds according to the invention possess valuable pharmacological properties and can be used for the prevention and/or treatment of various diseases and disease-induced states in humans and animals.

The compounds according to the invention are potent selective dual V1a/V2 receptor antagonists, which inhibit vasopressin activity in vitro and in vivo.

The compounds according to the invention are particularly suitable for the prophylaxis and/or treatment of cardiovascular diseases. In this connection, the following may for example and preferably be mentioned as target indications: acute and chronic cardiac insufficiency, arterial hypertension, coronary heart disease, stable and unstable angina pectoris, myocardial ischaemia, myocardial infarction, shock, arteriosclerosis, atrial and ventricular arrhythmias, transitory and ischaemic attacks, stroke, inflammatory cardiovascular diseases, peripheral and cardiac vascular diseases, peripheral circulation disorders, arterial pulmonary hypertension, spasms of the coronary arteries and peripheral arteries, thromboses, thromboembolic diseases, oedema formation such as for example pulmonary oedema, cerebral oedema, renal oedema or cardiac insufficiency-related oedema, and restenoses for example after thrombolysis treatments, percutaneous-transluminal angioplasties (PTA), transluminal coronary angioplasties (PTCA), heart transplants and bypass operations.

In the sense of the present invention, the term cardiac insufficiency also includes more specific or related disease forms such as right cardiac insufficiency, left cardiac insufficiency, global insufficiency, ischaemic cardiomyopathy, dilatative cardiomyopathy, congenital heart defects, heart valve defects, cardiac insufficiency with heart valve defects, mitral valve stenosis, mitral valve insufficiency, aortic valve stenosis, aortic valve insufficiency, tricuspidal stenosis, tricuspidal insufficiency, pulmonary valve stenosis, pulmonary valve insufficiency, combined heart valve defects, heart muscle inflammation (myocarditis), chronic myocarditis, acute myocarditis, viral myocarditis, diabetic cardiac insufficiency, alcohol-toxic cardiomyopathy, cardiac storage diseases, diastolic cardiac insufficiency and systolic cardiac insufficiency.

Furthermore, the compounds according to the invention are suitable for use as a diuretic for the treatment of oedemas and in electrolyte disorders, in particular in hypervolaemic and euvolaemic hyponatraemia.

The compounds according to the invention are also suitable for the prophylaxis and/or treatment of polycystic kidney disease (PCKD) and syndrome of inappropriate ADH secretion (SIADH).

In addition, the compounds according to the invention can be used for the prophylaxis and/or treatment of liver cirrhosis, ascites, diabetes mellitus and diabetic complications such as for example neuropathy and nephropathy, acute and chronic kidney failure and chronic renal insufficiency.

Further, the compounds according to the invention are suitable for the prophylaxis and/or treatment of central nervous disorders such as anxiety states and depression, of glaucoma and of cancer, in particular of pulmonary tumours.

Furthermore, the compounds according to the invention can be used for the prophylaxis and/or treatment of inflammatory diseases, asthmatic diseases, chronic-obstructive respiratory tract diseases (COPD), pain conditions, prostatic hypertrophy, incontinence, bladder inflammation, hyperactive bladder, diseases of the adrenals such as for example phaeochromocytoma and adrenal apoplexy, diseases of the intestine such as for example Crohn's disease and diarrhoea, or of menstrual disorders such as for example dysmenorrhoea, or endometriosis.

A further object of the present invention is the use of the compounds according to the invention for the treatment and/or prophylaxis of diseases, in particular of the diseases mentioned above.

A father object of the present invention are the compounds according to the invention for use in a method for the treatment and/or prophylaxis of acute and chronic cardiac insufficiency, hypervolaemic and envolaemic hyponatraemia, liver cirrhosis, ascites, oedemas, and the syndrome of inadequate ADH secretion (SIADH).

A further object of the present invention is the use of the compounds according to the invention for the production of a medicament for the treatment and/or prophylaxis of diseases, in particular of the diseases mentioned above.

A further object of the present invention is a method for the treatment and/or prophylaxis of diseases, in particular of the diseases mentioned above, with the use of an effective quantity of at least one of the compounds according to the invention.

The compounds according to the invention can be used alone or if necessary in combination with other active substances. A further object of the present invention are medicaments which contain at least one of the compounds according to the invention and one or more other active substances, in particular for the treatment and/or prophylaxis of the diseases mentioned above. As combination active substances suitable for this, the following may for example and preferably be mentioned:
organic nitrates and NO donors, such as for example sodium nitroprusside, nitroglycerine, isosorbide mononitrate, isosorbide dinitrate, molsidomine or SIN-1, and inhalational NO;
diuretics, in particular loop diuretics and thiazides and thiazide-like diuretics;
positive-inotropically active compounds, such as for example cardiac glycosides (digoxin), and beta-adrenergic and dopaminergic agonists such as isoproterenol, adrenalin, noradrenalin, dopamine and dobutamine;
compounds which inhibit the degradation of cyclic guanosine monophosphate (cGMP) and/or cyclic adenosine monophosphate (cAMP), such as for example inhibitors of phosphodiesterases (PDE) 1, 2, 3, 4 and/or 5, in particular PDE 5 inhibitors such as sildenafil, vardenafil and tadalafil, and PDE 3 inhibitors such as aminone and milrinone;
natriuretic peptides such as for example “atrial natriuretic peptide” (ANP, anaritide), “B-type natriuretic peptide” or “brain natriuretic peptide” (BNP, nesiritide), “C-type natriuretic peptide” (CNP) and urodilatin;
calcium sensitisers, such as for example and preferably levosimendan;
NO- and haem-independent activators of guanylate cyclase, such as in particular the compounds described in WO 01/19355, WO 01/19776, WO 01/19778, WO 01/19780, WO 02/070462 and WO 02/070510;
NO-independent, but haem-dependent stimulators of guanylate cyclase, such as in particular riociguat and the compounds described in WO 00/06568, WO 00/06569, WO 02/42301 and WO 03/095451;
inhibitors of human neutrophil elastase (HNE), such as for example sivelestat or DX-890 (reltran);
compounds inhibiting the signal transduction cascade, such as for example tyrosine kinase inhibitors, in particular sorafenib, imatinib, gefitinib and erlotinib;
compounds influencing the energy metabolism of the heart, such as for example and preferably etomoxir, dichloracetate, ranolazine or trimetazidine;
agents with antithrombotic action, for example and preferably from the group of the thrombo-cyte aggregation inhibitors, anticoagulants or profibrinolytic substances;
blood pressure-lowering active substances, for example and preferably from the group of the calcium antagonists, angiotensin AII antagonists, ACE inhibitors, vasopeptidase inhibitors, inhibitors of neutral endopeptidase, endothelin antagonists, renin inhibitors, alpha receptor blockers, beta receptor blockers, mineralocorticoid receptor antagonists and rho-kinase inhibitors; and/or
active substances modifying fat metabolism, for example and preferably from the group of the thyroid receptor agonists, cholesterol synthesis inhibitors such as for example and preferably HMG-CoA reductase or squalene synthesis inhibitors, ACAT inhibitors, CETP inhibitors, MTP inhibitors, PPAR-alpha, PPAR-gamma and/or PPAR-delta agonists, cholesterol absorption inhibitors, lipase inhibitors, polymeric gallic acid adsorbers, gallic acid reabsorption inhibitors and lipoprotein(a) antagonists.

In a preferred embodiment of the invention, the compounds according to the invention are administered in combination with a diuretic, such as for example and preferably furosemid, bumetanid, torsemid, bendroflumethiazid, chlorthiazid, hydrochlorthiazid, hydroflumethiazid, methyclothiazid, polythiazid, trichlormethiazid, chlorthalidon, indapamid, metolazon, quinethazon, acetazolamid, dichlorophenamid, methazolamid, glycerine, isosorbide, mannitol, amilorid or triamteren.

Agents with antithrombotic action are understood preferably to mean compounds from the group of the thrombocyte aggregation inhibitors, anticoagulants or profibrinolytic substances.

In a preferred embodiment of the invention, the compounds according to the invention are administered in combination with a thrombocyte aggregation inhibitor, such as for example and preferably aspirin, clopidogrel, ticlopidine or dipyridamol.

In a preferred embodiment of the invention, the compounds according to the invention are administered in combination with a thrombin inhibitor, such as for example and preferably ximela-gatran, melagatran, bivalirudin or clexane.

In a preferred embodiment of the invention, the compounds according to the invention are administered in combination with a GPIIb/IIIa antagonist, such as for example and preferably tirofiban or abciximab.

In a preferred embodiment of the invention, the compounds according to the invention are administered in combination with a factor Xa inhibitor, such as for example and preferably riva-roxaban (BAY 59-7939), DU-176b, apixaban, otamixaban, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds according to the invention are administered in combination with heparin or a low molecular weight (LMW) heparin derivative.

In a preferred embodiment of the invention, the compounds according to the invention are administered in combination with a vitamin K antagonist, such as for example and preferably coumarin.

Blood pressure-lowering agents are understood preferably to mean compounds from the group of the calcium antagonists, angiotensin AII antagonists, ACE inhibitors, vasopeptidase inhibitors, inhibitors of neutral endopeptidase, endothelin antagonists, renin inhibitors, alpha receptor blockers, beta receptor blockers, mineralocorticoid receptor antagonists, rho-kinase inhibitors and diuretics.

In a preferred embodiment of the invention, the compounds according to the invention are administered in combination with a calcium antagonist, such as for example and preferably nife-dipin, amlodipin, verapamil or diltiazem.

In a preferred embodiment of the invention, the compounds according to the invention are administered in combination with an angiotensin AII antagonist, such as for example and preferably losartan, candesartan, valsartan, telmisartan or embusartan.

In a preferred embodiment of the invention, the compounds according to the invention are administered in combination with an ACE inhibitor, such as for example and preferably enalapril, captopril, lisinopril, ramipril, delapril, fosinopril, quinopril, perindopril or trandopril.

In a preferred embodiment of the invention, the compounds according to the invention are administered in combination with a vasopeptidase inhibitor or inhibitor of neutral endopeptidase (NEP).

In a preferred embodiment of the invention, the compounds according to the invention are administered in combination with an endothelin antagonist, such as for example and preferably bosentan, darusentan, ambrisentan or sitaxsentan.

In a preferred embodiment of the invention, the compounds according to the invention are administered in combination with a renin inhibitor, such as for example and preferably aliskiren, SPP-600 or SPP-800.

In a preferred embodiment of the invention, the compounds according to the invention are administered in combination with an alpha-1 receptor blocker, such as for example and preferably prazosin.

In a preferred embodiment of the invention, the compounds according to the invention are administered in combination with a beta receptor blocker, such as for example and preferably propranolol, atenolol, timolol, pindolol, alprenolol, oxprenolol, penbutolol, bupranolol, meti-pranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds according to the invention are administered in combination with a mineralocorticoid receptor antagonist, such as for example and preferably spironolactone, eplerenon, canrenon or potassium canrenoate.

In a preferred embodiment of the invention, the compounds according to the invention are administered in combination with a rho-kinase inhibitor, such as for example and preferably fasu-dil, Y-27632, SLx-2119, BF-66851, BF-66852, BF-66853, KI-23095 or BA-1049.

Fat metabolism-modifying agents are understood preferably to mean compounds from the group of the CETP inhibitors, thyroid receptor agonists, cholesterol synthesis inhibitors such as HMG-CoA reductase or squalene synthesis inhibitors, ACAT inhibitors, MTP inhibitors, PPAR-alpha, PPAR-gamma and/or PPAR-delta agonists, cholesterol absorption inhibitors, polymeric gallic acid adsorbers, gallic acid reabsorption inhibitors, lipase inhibitors and lipoprotein(a) antagonists.

In a preferred embodiment of the invention, the compounds according to the invention are administered in combination with a CETP inhibitor, such as for example and preferably dalcetrapib, BAY 60-5521, anacetrapib or CETP-vaccine (CETi-1).

In a preferred embodiment of the invention, the compounds according to the invention are administered in combination with a thyroid receptor agonist, such as for example and preferably D-thyroxine, 3,5,3′-triiodothyronine (T3), CGS 23425 or axitirome (CGS 26214).

In a preferred embodiment of the invention, the compounds according to the invention are administered in combination with an HMG-CoA reductase inhibitor from the class of the statins, such as for example and preferably lovastatin, simvastatin, pravastatin, fluvastatin, atorvastatin, rosuvastatin or pitavastatin.

In a preferred embodiment of the invention, the compounds according to the invention are administered in combination with a squalene synthesis inhibitor, such as for example and preferably BMS-188494 or TAK-475.

In a preferred embodiment of the invention, the compounds according to the invention are administered in combination with an ACAT inhibitor, such as for example and preferably avasi-mibe, melinamide, pactimibe, eflucimibe or SMP-797.

In a preferred embodiment of the invention, the compounds according to the invention are administered in combination with an MTP inhibitor, such as for example and preferably implitapide, BMS-201038, R-103757 or JTT-130.

In a preferred embodiment of the invention, the compounds according to the invention are administered in combination with a PPAR-gamma agonist, such as for example and preferably pio-glitazone or rosiglitazone.

In a preferred embodiment of the invention, the compounds according to the invention are administered in combination with a PPAR-delta agonist, such as for example and preferably GW-501516 or BAY 68-5042.

In a preferred embodiment of the invention, the compounds according to the invention are administered in combination with a cholesterol absorption inhibitor, such as for example and preferably ezetimibe, tiqueside or pamaqueside.

In a preferred embodiment of the invention, the compounds according to the invention are administered in combination with a lipase inhibitor, such as for example and preferably orlistat.

In a preferred embodiment of the invention, the compounds according to the invention are administered in combination with a polymeric gallic acid adsorber, such as for example and preferably cholestyramine, colestipol, colesolvam, cholestagel or colestimid.

In a preferred embodiment of the invention, the compounds according to the invention are administered in combination with a gallic acid reabsorption inhibitor, such as for example and preferably ASBT (=IBAT) inhibitors such as for example AZD-7806, S-8921, AK-105, BARI-1741, SC-435 or SC-635.

In a preferred embodiment of the invention, the compounds according to the invention are administered in combination with a lipoprotein(a) antagonist, such as for example and preferably gemcabene calcium (CI-1027) or nicotinic acid.

A further object of the present invention are medicaments which contain at least one compound according to the invention, usually together with one or more inert, non-toxic, pharmaceutically suitable additives, and the use thereof for the aforesaid purposes.

The compounds according to the invention can act systemically and/or locally. For this purpose, they can be administered in a suitable manner, such as for example by the oral, parenteral, pulmonary, nasal, sublingual, lingual, buccal, rectal, dermal, transdermal, conjunctival or aural routes or as an implant or stent.

For these administration routes, the compounds according to the invention can be administered in suitable administration forms.

For oral administration, administration forms which function according to the state of the art, releasing the compounds according to the invention rapidly and/or in a modified manner, which contain the compounds according to the invention in crystalline and/or amorphized and/or dissolved form, such as for example tablets (uncoated or coated tablets, for example with gastric juice-resistant or delayed dissolution or insoluble coatings, which control the release of the compound according to the invention), tablets rapidly disintegrating in the oral cavity or films/wafers, films/lyophilisates, capsules (for example hard or soft gelatine capsules), dragees, granules, pellets, powders, emulsions, suspensions, aerosols or solutions are suitable.

Parenteral administration can be effected omitting an absorption step (e.g. intravenous, intra-arterial, intracardial, intraspinal or intralumbar administration) or involving absorption (e.g. intra-muscular, subcutaneous, intracutaneous, percutaneous or intraperitoneal administration). Suitable administration forms for parenteral administration include injection and infusion preparations in the form of solutions, suspensions, emulsions, lyophilisates or sterile powders.

For the other administration routes, for example inhalation formulations (including powder inhalers and nebulisers), nasal drops, solutions or sprays, tablets for lingual, sublingual or buccal administration, tablets, films/wafers or capsules, suppositories, oral or ophthalmic preparations, vaginal capsules, aqueous suspensions (lotions, shakable mixtures), lipophilic suspensions, ointments, creams, transdermal therapeutic systems (e.g. plasters), milk, pastes, foams, dusting powders, implants or stents are suitable.

Oral or parenteral administration, in particular oral and intravenous administration, are preferred.

The compounds according to the invention can be converted into the stated administration forms. This can be effected in a manner known per se by mixing with inert, non-toxic, pharmaceutically suitable additives. These additives include carriers (for example microcrystalline cellulose, lactose, mannitol), solvents (e.g. liquid polyethylene glycols), emulsifiers and dispersants or wetting agents (for example sodium dodecylsulphate, polyoxysorbitan oleate), binders (for example polyvinylpyrrolidone), synthetic and natural polymers (for example albumin), stabilizers (e.g. antioxidants such as for example ascorbic acid), colourants (e.g. inorganic pigments such as for example iron oxides) and flavour or odour correctors.

In general, to achieve effective results in parenteral administration it has been found advantageous to administer quantities of about 0.001 to 10 mg/kg, preferably about 0.01 to 1 mg/kg body weight. In oral administration, the dosage is about 0.01 bis 100 mg/kg, preferably about 0.01 to 20 mg/kg and quite especially preferably 0.1 to 10 mg/kg body weight.

Nonetheless it can sometimes be necessary to deviate from the said quantities, namely depending on body weight, administration route, individual response to the active substance, nature of the preparation and time or interval at which administration takes place. Thus in some cases it can be sufficient to manage with less than the aforesaid minimum quantity, while in other cases the stated upper limit must be exceeded. In the event of administration of larger quantities, it may be advisable to divide these into several individual administrations through the day.

The following practical examples illustrate the invention. The invention is not limited to the examples.

Unless otherwise stated, the percentages stated in the following tests and examples are percent by weight, parts are parts by weight, and solvent ratios, dilution ratios and concentration information about liquid/liquid solutions are each based on volume.

### A. EXAMPLES

### Abbreviations

BOC tert-butoxycarbonyl
CI chemical ionization (in MS)
DCI direct chemical ionization (in MS)
DME 1,2-dimethoxyethane
DMF dimethylformamide
DMSO dimethyl sulphoxide
EDC N′-(3-dimethylaminopropyl)-N-ethylcarbodiimide hydrochloride
eq. equivalent(s)
ESI electrospray ionization (in MS)
GC/MS gas chromatography-coupled mass spectrometry
sat. saturated
h hour(s)
HOBt 1-hydroxy-1H-benzotriazole hydrate
HPLC high pressure, high performance liquid chromatography
HV high vacuum
LC/MS liquid chromatography-coupled mass spectrometry
LDA lithium diisopropylamide
LiHMDS lithium hexamethyldisilazane
min(s) minute(s)
MS mass spectrometry
MTBE methyl tert-butyl ether
NMR nuclear magnetic resonance spectrometry
rac racemic/racemate
R_{f} retention factor (in thin layer chromatography on silica gel)
RT room temperature
Rₜ retention time (in HPLC)
THF tetrahydrofuran
TMOF trimethyl orthoformate
UV ultraviolet spectrometry
v/v volume to volume ratio (of a solution)

### LC/MS, HPLC and GC/MS Methods:

Method 1: MS instrument type: Micromass ZQ; HPLC instrument type: Waters Alliance 2795; column. Phenomenex Synergi 2.5μ MAX-RP 100A Mercury 20 mm×4 mm; eluent A: 1 l water+0.5 ml 50% formic acid, eluent B: 1 l acetonitrile+0.5 ml 50% formic acid; gradient: 0.0 min 90% A→0.1 min 90% A→3.0 min 5% A→4.0 min 5% A→4.01 min 90% A; flow rate: 2 ml/min; oven: 50° C.; UV detection: 210 nm.

Method 2: MS instrument type: Waters (Micromass) Quattro Micro; HPLC instrument type: Agilent 1100 series; column: Thermo Hypersil GOLD 3μ 20×4 mm; eluent A: 1 l water+0.5 ml 50% formic acid; eluent B: 1 l acetonitrile+0.5 ml 50% formic acid; gradient: 0.0 min 100% A→3.0 min 10% A→4.0 min 10% A→4.01 min 100% A (flow 2.5 ml)→5.00 min 100% A; oven: 50° C.; flow rate: 2 ml/min; UV detection: 210 nm

Method 3: Instrument: Micromass Quattro Premier with Waters UPLC Acquity; column: Thermo Hypersil GOLD 1.9μ 50×1 mm; eluent A: 1 l water+0.5 ml 50% formic acid, eluent B: 1 l acetonitrile+0.5 ml 50% formic acid; gradient: 0.0 min 90% A→0.1 min 90% A→1.5 min 10% A→2.2 min 10% A; oven: 50° C.; flow rate: 0.33 ml/min; UV detection: 210 nm.

Method 4: Instrument: Waters ACQUITY SQD HPLC System; column: Waters Acquity UPLC HSS T3 1.8μ 50×1 mm; eluent A: 1 l water+0.25 ml 99% formic acid; eluent B: 1 l acetonitrile+0.25 ml 99% formic acid; gradient: 0.0 min 90% A→1.2 min 5% A→2.0 min 5% A; oven: 50° C.; flow rate: 0.40 ml/min; UV detection: 210-400 nm.

Method 5: Instrument: Waters ACQUITY SQD UPLC System; column: Waters Acquity UPLC HSS T3 1.8μ 50×1 mm; eluent A: 1 l water+0.25 ml 99% formic acid; eluent B: 1 l acetonitrile+0.25 ml 99% formic acid; gradient: 0.0 min 90% A→1.2 min 5% A→2.0 min 5% A; oven: 50° C.; flow rate: 0.40 ml/min; UV detection: 210-400 nm.

Method 6: MS instrument type: Micromass ZQ; HPLC instrument type: HP 1100 Series; UV DAD; column. Phenomenex Gemini 3μ 30 mm×3.00 mm; eluent A: 1 l water+0.5 ml 50% formic acid; eluent B: 1 l acetonitrile+0.5 ml 50% formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

Method 7: (preparative HPLC): Column Grom-Sil 120 ODS-4HE 10 μm, 250 mm×40 mm; Eluent: A=water, B=acetonitrile; Programme: 0-6 min: 5% B; 6-27 min: Gradient 5% to 95% B; 27-43 min: 95% B; 43-45 min: 5% B. Flow rate: 50 ml/min; Column temperature: RT; UV detection: 210 nm.

Method 8: (chiral preparative HPLC): Chiral stationary silica gel phase based on the selector poly-(N-methacryloyl-D-leucine-dicyclopropylmethylamide); Column: 600 mm×30 mm, Flow rate: 50 ml/min, Temperature: 24° C.; UV detector 260 nm. Eluent: isohexane/ethyl acetate 50:50.

Method 9: (analyical preparative HPLC): Chiral stationary silica gel phase based on the selector poly(N-methacryloyl-D-leucine-dicyclopropylmethylamide); Column: 250 mm×4.6 mm, Eluent: ethyl acetate 100%, Flow rate: 2 ml/min, Temperature: 24° C.; UV detector 265 nm.

Method 10 (preparative HPLC): column. Grom-Sil 120 ODS-4HE, 10 μm, SNo. 3331, 250 mm×30 mm Eluent A: formic acid 0.1% in water, eluent B: acetonitrile; flow rate: 50 ml/min Programme: 0-3 min: 10% B; 3-27 min: Gradient to 95% B; 27-34 min: 95% B; 34.01-38 min: 10% B.

Method 11: (chiral preparative HPLC): Chiral stationary silica gel phase based on the selector poly(N-methacryloyl-L-leucine-(+)-3-pinancemethylamide); Column: 600 mm×30 mm, Flow rate: 80 ml/min, Temperature: 24° C.; UV detector 2650 nm. Various eluents:
Method 11a: Eluent: 100% ethyl acetate
Method 11b: Eluent: Gradient 0-15 min from isohexane/ethyl acetate 40:60 to 100% ethyl acetate; 15-25 min: 100% ethyl acetate.
Method 11c: Eluent: isohexane/ethyl acetate 10:90.

Method 12: (chiral analytical HPLC): Chiral stationary silica gel phase based on the selector poly-(N-methacryloyl-L-leucine-(+)-3-pinanemethylamide); Column: 250 mm×4.6 mm, Temperature 24° C.; UV dector 265 nm. Flow rate: 2 ml/min. Various eluents:
Method 12a: Eluent: 100% ethyl acetate.
Method 12b: Eluent: isohexane/ethyl acetate 50:50.

Method 13: (preparative HPLC): Column Grom-Sil 120 ODS-4HE 10 μm, 250 mm×30 mm; Eluent: A=water, B=acetonitrile; Gradient: 0.0 min 10% B, 3 min to 30 min: Gradient 10% to 95% B, 42 min 95% B, 42.01 min 10% B, 45 min 10% B; Flow rate: 50 ml/min; Column temperature: RT; UV detection: 210 nm.

Method 14 (preparative HPLC): Column: Reprosil C18, 10 μm, SNo. 3500, 250 mm×30 mm Eluent A: formic acid 0.1% in water, Eluent B: methanol; Flow rate: 50 ml/min Programme: 0-4.25 min: 40% B; 4.25-4.50 min: Gradient to 60% B; 4.50-17 min: Gradient to 100% B; 17-19.50 min: 100% B; 19.51-19.75 min: Gradient to 40% B 19.75-20.5 min: 40% B.

Method 15 (preparative HPLC): Column: Reprosil C18, 10 μm, SNo. 3500, 250 mm×30 mm Eluent A: formic acid 0.1% in water, Eluent B: acetonitrile; Flow rate: 50 ml/min Programme: 0-6 min: 10% B; 6-27 min: Gradient to 95% B; 27-43 min: 95% B; 43.01-44 min: 10% B.

### Starting Compounds and Intermediates

### Example 1A

### Ethyl N-({2-[(4-chlorophenyl)carbonyl]hydrazinyl}carbonyl)glycinate

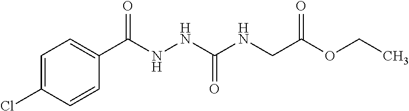

A suspension of 12.95 g (75.9 mmol) of 4-chlorobenzhydrazide in 50 ml of dry THF was introduced at 50° C. and admixed dropwise with a solution of 10.0 g (77.5 mmol) of ethyl 2-isocyanatoacetate in 100 ml of dry THF. First of all a solution formed, and then a precipitate was produced. After the end of the addition, the mixture was stirred at 50° C. for 2 h more, then left to stand overnight at RT. The crystals were isolated by filtration, washed with a little diethyl ether and dried in an HV. This gave 21.43 g (89% of theory) of the title compound.

LC/MS [Method 1]: Rₜ=1.13 min; m/z=300 (M+H)⁺

¹H NMR (DMSO-d₆, 400 MHz): δ=□□ 10.29 (s, 1H), 8.21 (s, 1H), 7.91 (d, 2H), 7.57 (d, 2H), 6.88 (br.s, 1H), 4.09 (q, 2H), 3.77 (d, 2H), 1.19 (t, 3H)

### Example 2A

### [3-(4-Chlorophenyl)-5-oxo-1,5-dihydro-4H-1,2,4-triazol-4-yl]acetic acid

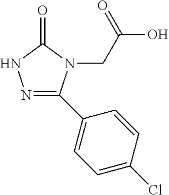

Of the compound from Example 1A, 21.43 g (67.93 mmol) were admixed with 91 ml of a 3N aqueous sodium hydroxide solution and heated at reflux overnight. After cooling to RT, the mixture was adjusted to a pH of 1 by slow addition of approximately 20% strength hydrochloric acid. The precipitated solid was isolated by filtration, washed with water and dried at 60° C. under reduced pressure. Yield: 17.55 g (90% of theory, approximately 88% purity).

LC/MS [Method 1]: Rₜ=0.94 min; m/z=254 (M+H)⁺

¹H NMR (DMSO-d₆, 400 MHz): δ=13.25 (br.s, 1H), 12.09 (s, 1H), 7.65-7.56 (m, 4H), 4.45 (s, 2H).

### Example 3A

### 5-(4-Chlorophenyl)-4-(3,3,3-trifluoro-2-oxopropyl)-2,4-dihydro-3H-1,2,4-triazol-3-one (or, in hydrate form: 5-(4-chlorophenyl)-4-(3,3,3-trifluoro-2,2-dihydroxypropyl)-2,4-dihydro-3H-1,2,4-triazol-3-one)

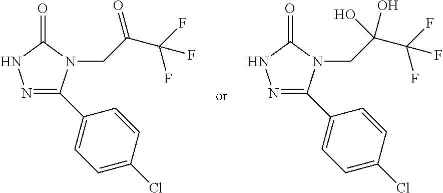

Of the compound from Example 2A, 5 g (16.36 mmol) were dissolved under argon in 200 ml of pyridine and then admixed with 17.18 g (81.8 mmol) of trifluoroacetic anhydride. The temperature rose to about 35° C. After 30 min, the pyridine was removed on a rotary evaporator and the residue was diluted with 1.5 l of 0.5N hydrochloric acid. This mixture was heated to 70° C. and then filtered while hot. The solid was washed with a little water. The entire filtrate was extracted three times with ethyl acetate. The combined organic phases were washed with water, then with a saturated aqueous sodium hydrogen carbonate solution, then with a saturated aqueous sodium chloride solution, dried over sodium sulphate and freed from the solvent on a rotary evaporator. The residue was dried under HV. Yield: 3.56 g (68% of theory) of the title compound in hydrate form.

LC/MS [Method 1]: Rₜ=1.51 min; m/z=306 (M+H)⁺ and 324 (M+H)⁺ (ketone and hydrate)

¹H NMR (DMSO-d₆, 400 MHz): δ=□□ 12.44 (s, 1H), 7.72 (d, 2H), 7.68 (br.s, 2H), 7.61 (d, 2H), 3.98 (s, 2H).

### Example 4A

### 5-(4-Chlorophenyl)-4-(3,3,3-trifluoro-2-hydroxypropyl)-2,4-dihydro-3H-1,2,4-triazol-3-one

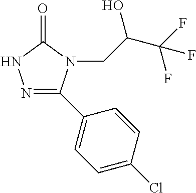

Of the compound from Example 3A, 3.56 g (11 mmol) were dissolved in 100 ml of methanol and admixed, with ice cooling, with 3.75 g (99 mmol) of sodium borohydride (gas evolution). After 1.5 h, 200 ml of 1M hydrochloric acid were slowly added. The methanol was removed on a rotary evaporator and the residue was diluted with 500 ml of water and extracted three times with ethyl acetate. The combined organic phases were washed with saturated aqueous sodium hydrogen carbonate solution, then with saturated aqueous sodium chloride solution, dried over sodium sulphate and freed from the solvent on a rotary evaporator. The residue was dried under an HV. This gave 3.04 g (90% of theory) of the title compound.

LC/MS [Method 2]: Rₜ=1.80 min; m/z=308 (M+H)⁺.

¹H NMR (DMSO-d₆, 400 MHz): δ=□□ 12.11 (s, 1H), 7.75 (d, 2H), 7.62 (d, 2H), 6.85 (d, 1H), 4.34-4.23 (m, 1H), 3.92 (dd, 1H), 3.77 (dd, 1H).

### Example 5A

### Methyl [3-(4-chlorophenyl)-5-oxo-4-(3,3,3-trifluoro-2-hydroxypropyl)-4,5-dihydro-1H-1,2,4-triazol-1-yl]acetate

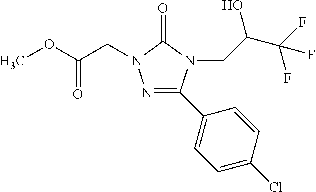

Of the compound from Example 4A, 3.04 g (9.9 mmol) were dissolved in 100 ml of acetonitrile and admixed with 1.07 g (9.9 mmol) of methyl chloroacetate, 2.73 g (19.8 mmol) of potassium carbonate and a small spatula-tipful of potassium iodide. The reaction mixture was heated at reflux for 1 h, left to cool to RT and filtered. The filtrate was freed from the volatile components on a rotary evaporator and the residue was dried in an HV. Yield: 3.70 g (89% of theory) of the title compound in approximately 90% purity.

LC/MS [Method 3]: Rₜ=1.10 min; m/z=380 (M+H)⁺.

¹H NMR (DMSO-d₆, 400 MHz): δ=□□ 7.78 (d, 2H), 7.64 (d, 2H), 6.91 (d, 1H), 4.72 (s, 2H), 4.16-4.35 (m, 1H), 3.99 (dd, 1H), 3.84 (dd, 1H), 3.70 (s, 3H).

The racemic compound from Example 5A was resolved by preparative HPLC on a chiral phase into its enantiomers Example 6A and Example 7A, as described in WO 2007/134862.

Column: chiral silica gel phase based on the selector poly(N-methacryloyl-L-isoleucine-3-pentylamide), 430 mm×40 mm; eluent: step gradient isohexane/ethyl acetate 1:1→ethyl acetate→isohexane/ethyl acetate 1:1; flow rate: 50 ml/min; temperature: 24° C.; UV detection: 260 nm.

This gives, from 3.6 g of racemic compound from Example 5A (dissolved in 27 ml of ethyl acetate and 27 ml of isohexane and separated into three portions by the column), 1.6 g of the enantiomer 1 which elutes first (Example 6A), and 1.6 g of the enantiomer 2 which elutes subsequently (Example 7A).

### Example 6A

### Methyl {3-(4-chlorophenyl)-5-oxo-4-[(2S)-3,3,3-trifluoro-2-hydroxypropyl]-4,5-dihydro-1H-1,2,4-triazol-1-yl}acetate (Enantiomer I)

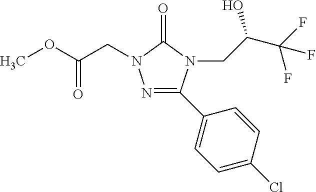

First-eluting enantiomer from the racemate resolution of Example 5A.

Rₜ=3.21 min [column: chiral silica gel phase based on the selector poly(N-methacryloyl-L-isoleucine-3-pentylamide), 250 mm×4.6 mm; eluent: isohexane/ethyl acetate 1:1; flow rate: 1 ml/min; UV detection: 260 nm].

### Example 7A

### Methyl {3-(4-chlorophenyl)-5-oxo-4-[(2R)-3,3,3-trifluoro-2-hydroxypropyl]-4,5-dihydro-1H-1,2,4-triazol-1-yl}acetate (Enantiomer II)

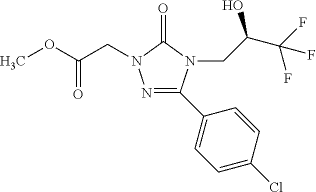

Last-eluting enantiomer from the racemate resolution of Example 5A.

Rₜ=4.48 min [column: chiral silica gel phase based on the selector poly(N-methacryloyl-L-isoleucine-3-pentylamide), 250 mm×4.6 mm; eluent: isohexane/ethyl acetate 1:1; flow rate: 1 ml/min; UV detection: 260 nm].

### Example 8A

### {3-(4-Chlorophenyl)-5-oxo-4-[(2S)-3,3,3-trifluoro-2-hydroxypropyl]-4,5-dihydro-1H-1,2,4-triazol-1-yl}acetic acid

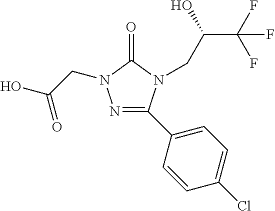

The enantiomerically pure ester from Example 6A (1.6 g, 4.21 mmol) was dissolved in 77 ml of methanol and admixed with 17 ml of a 1M solution of lithium hydroxide in water. The mixture was stirred at RT for 1 h and then concentrated on a rotary evaporator. The residue was diluted with 100 ml of water and acidified to a pH of 1-2 using 1N hydrochloric acid. The precipitated product was isolated by filtration, washed in succession with water and cyclohexane and sucked dry. After further drying in an HV, the title compound (1.1 g, 71% of theory) was obtained.

[α]_{D}²⁰=+3.4° (methanol, c=0.37 g/100 ml)

LC/MS [Method 1]: Rₜ=1.51 min; m/z=366 (M+H)⁺

¹H-NMR (400 MHz, DMSO-d₆): δ=3.84 (dd, 1H), 4.00 (dd, 1H), 4.25 (m, 1H), 4.58 (s, 2H), 6.91 (d, 1H), 7.63 (d, 2H), 7.78 (d, 2H), 13.20 (br. s, 1H).

### Example 9A

### {3-(4-Chlorophenyl)-5-oxo-4-[(2R)-3,3,3-trifluoro-2-hydroxypropyl]-4,5-dihydro-1H-1,2,4-triazol-1-yl}acetic acid

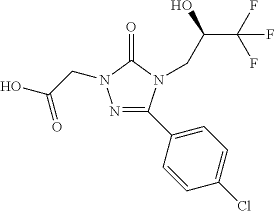

In the same way as for Example 8A, the title compound was obtained from Example 7A.

[α]_{D}²⁰=−4.6° (methanol, c=0.44 g/100 ml)

LC/MS [Method 1]: Rₜ=1.53 min; m/z=366 (M+H)⁺

¹H-NMR (400 MHz, DMSO-d₆): δ=3.84 (dd, 1H), 4.00 (dd, 1H), 4.25 (m, 1H), 4.58 (s, 2H), 6.91 (d, 1H), 7.63 (d, 2H), 7.78 (d, 2H), 13.20 (br. s, 1H).

### Example 10A

### (2R)-2-[({3-(4-Chlorophenyl)-5-oxo-4-[(2S)-3,3,3-trifluoro-2-hydroxypropyl]-4,5-dihydro-1H-1,2,4-triazol-1-yl}acetyl)amino]-2-[3-(trifluoromethyl)phenyl]propanoic acid

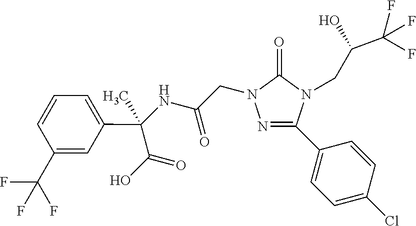

A quantity of 3.77 g (10.3 mmol) of the compound from Example 8A and 1.47 g of HOBt (10.31 mmol) in 60 ml of DMF were admixed with 1.98 g (10.31 mmol) of EDC and stirred for 20 minutes. The resulting solution was added dropwise to a suspension of 3.06 g (11.35 mmol) of (2R)-2-amino-2-[3-(trifluoromethyl)phenyl]propanoic hydrochloride (from Netchem, New Brunswick, N.J. 08901, USA) and 2.16 ml (12.4 mmol) of N,N-diisopropylethylamine in 60 ml of DMF. Following the addition, the mixture was stirred at RT for 1 hour more, then admixed with 500 ml of 0.5 N hydrochloric acid and extracted three times with ethyl acetate. The combined organic phases were washed in succession three times with water and once with saturated aqeuous sodium chloride solution, then dried over sodium sulphate. The solvent was removed on a rotary evaporator and the residue was dried under a high vacuum. This gave 6.60 g (88% of theory, purity 80%) of the title compound.

LC-MS [Method 3]: Rₜ=1.22 min; MS [ESpos]: m/z=581 (M+H)⁺

¹H-NMR (400 MHz, DMSO-d₆): δ [ppm]=1.85 (s, 3H), 3.82 (dd, 1H), 3.96 (dd, 1H), 4.22-4.33 (m, 1H), 4.59 (s, 2H), 6.92 (d, 1H), 7.57-7.70 (m, 4H), 7.73-7.81 (m, 4H), 8.80 (s, 1H), 13.11 (br. s., 1H).

### Example 11A

### (2R)-2-[({3-(4-Chlorophenyl)-5-oxo-4-[(2S)-3,3,3-trifluoro-2-hydroxypropyl]-4,5-dihydro-1H-1,2,4-triazol-1-yl}acetyl)amino]-2-[3-(trifluoromethyl)phenyl]propanamide

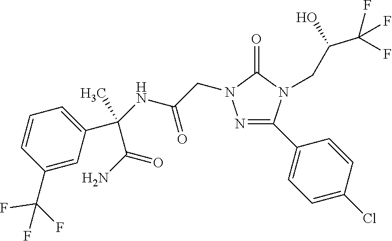

A quantity of 3.44 g (4.20 mmol) of the compound from Example 10A and 1.02 g of HOBt (7.57 mmol) in 40 ml DMF were admixed with 1.45 g (7.57 mmol) of EDC and stirred for 30 minutes. The resulting solution was added dropwise to an ammonia solution (35% in water, 45 ml). Following the addition, the mixture was stirred at RT for 20 minutes and then concentrated on a rotary evaporator. The residue was admixed with 500 ml of water and extracted with three times 250 ml of ethyl acetate. The combined organic phases were washed in succession twice with 1M hydrochloric acid, once with water, twice with saturated aqueous sodium hydrogen carbonate solution and once with saturated aqueous sodium chloride solution, then dried over sodium sulphate. The solvent was removed on the rotary evaporator and the residue was purified by preparative HPLC (Method 7). The product was dried under a high vacuum. This gave 2.30 g (94% of theory) of the title compound.

LC-MS [Method 3]: Rₜ=1.21 min; MS [ESpos]: m/z=580 (M+H)⁺

¹H-NMR (400 MHz, DMSO-d₆): δ [ppm]=1.88 (s, 3H), 3.82 (dd, 1H), 3.96 (dd, 1H), 4.21-4.33 (m, 1H), 4.58 (s, 2H), 6.89 (d, 1H), 7.33 (s, 1H), 7.41 (s, 1H), 7.57 (t, 1H), 7.61-7.65 (m, 3H), 7.70-7.78 (m, 4H), 8.63 (s, 1H).

### Example 12A

### 2-{3-(4-Chlorophenyl)-5-oxo-4-[(2S)-3,3,3-trifluoro-2-hydroxypropyl]-4,5-dihydro-1H-1,2,4-triazol-1-yl}-N-{(1R)-1-cyano-1-[3-(trifluoromethyl)phenyl]ethyl}acetamide

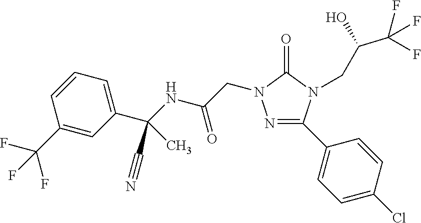

A quantity of 200 mg (0.345 mmol) of the compound from Example 11A was dissolved in 4 ml of dry THF, admixed with 61 μl of pyridine (0.76 mmol) and then with 102 μl of trifluoroacetic anhydride (0.72 mmol), and the resulting mixture was stirred at RT overnight. The volatile components were then removed on a rotary evaporator and the residue was purified by preparative HPLC (Method 10). This gave 157 mg (81% of theory) of the title compound.

LC-MS [Method 3]: Rₜ=1.32 min; MS [ESpos]: m/z=562 (M+H)⁺

¹H-NMR (400 MHz, DMSO-d₆): δ [ppm]=1.88 (s, 3H), 3.81 (dd, 1H), 3.94 (dd, 1H), 4.21-4.32 (m, 1H), 4.63 (s, 2H), 6.91 (d, 1H), 7.60-7.69 (m, 3H), 7.71-7.76 (m, 4H), 7.79 (d, 1H), 9.58 (s, 1H).

### Example 13A

### N-{(2R)-1-Amino-1-(hydroxyimino)-2-[3-(trifluoromethyl)phenyl]propan-2-yl}-2-{3-(4-chlorophenyl)-5-oxo-4-[(2S)-3,3,3-trifluoro-2-hydroxypropyl]-4,5-dihydro-1H-1,2,4-triazol-1-yl}acetamide

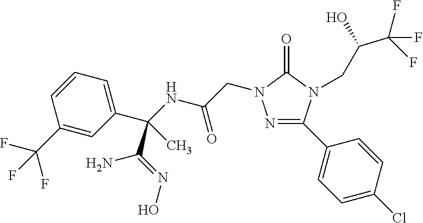

A quantity of 110 mg (196 μmol) of the compound from Example 12A were dissolved with 68 mg (0.98 mmol) of hydroxylamine hydrochloride and 136 μl of triethylamine (0.98 mmol) in 2.9 ml of DMSO and the mixture was stirred at 75° C. overnight. After cooling to RT, water was added and the mixture was extracted three times with ethyl acetate. The combined organic phases were washed with saturated aqueous sodium chloride solution, dried over sodium sulphate and freed from the volatile components on a rotary evaporator. The residue was taken up in a little DMSO and the product was purified by preparative HPLC (Method 10). This gave 43 mg (37% of theory) of the title compound.

LC-MS [Method 3]: Rₜ=1.19 min; MS [ESpos]: m/z=595 (M+H)⁺

¹H-NMR (400 MHz, DMSO-d₆): δ [ppm]=1.79 (s, 3H), 3.82 (dd, 1H), 3.96 (dd, 1H), 4.21-4.32 (m, 1H), 4.53-4.62 (m, 2H), 5.52-5.58 (m, 2H), 6.90 (d, 1H), 7.51-7.69 (m, 6H), 7.75 (d, 2H), 8.57 (s, 1H), 9.43 (s, 1H).

### Example 14A

### 2-{3-(4-Chlorophenyl)-5-oxo-4-[(2S)-3,3,3-trifluoro-2-hydroxypropyl]-4,5-dihydro-1H-1,2,4-triazol-1-yl}-N-{(2R)-1-hydrazino-1-oxo-2-[3-(trifluoromethyl)phenyl]propan-2-yl}acetamide

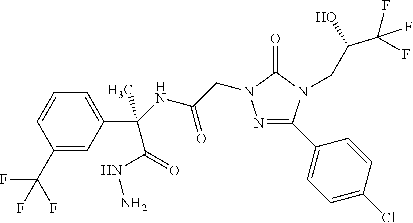

A quantity of 156 mg (0.27 mmol) of the compound from Example 10A was dissolved in 2 ml of acetonitrile, admixed with 72 mg (0.38 mmol) of EDC and 54 mg (0.38 mmol) of HOBt and stirred at RT for 20 minutes. The resulting solution was added dropwise to a solution, cooled to 0° C. beforehand, of 26 μl (0.54 mmol) of hydrazine hydrate and 6.8 μl (67 μmol) of cyclohexene in 2 ml of acetonitrile. Following the addition, the mixture was stirred for 30 minutes more, admixed with 2 ml of 1N hydrochloric acid and separated by preparative HPLC (Method 10). The appropriate fraction was concentrated on a rotary evaporator and dried under a high vacuum. This gave 100 mg (63% of theory) of the title compound.

LC-MS [Method 6]: Rₜ=2.22 min; MS [ESneg]: m/z=595 (M+H)⁺

¹H-NMR (400 MHz, DMSO-d₆): δ [ppm]=1.86 (s, 3H), 3.82 (dd, 1H), 3.96 (dd, 1H), 4.22-4.33 (m, 1H), 4.60 (s, 2H), 6.90 (d, 1H), 7.56 (t, 1H), 7.60-7.66 (m, 3H), 7.70-7.79 (m, 4H), 8.67 (s, 1H), 9.21 (br. s., 1H).

### Examples 15A and 16A

The compound N-2-amino-2-oxo-1-[3-(trifluoromethyl)phenyl]ethyl}-2-{3-(4-chlorophenyl)-5-oxo-4-[(2S)-3,3,3-trifluoro-2-hydroxypropyl]-4,5-dihydro-1H-1,2,4-triazol-1-yl-acetamide in the form of a diastereomer mixture was prepared in analogy to WO 2007/134862 (Example 509) and resolved into its diastereomers by chromatography on a chiral solid phase (Method 11a). The first-eluting diastereomer (Diastereomer 1) is described under Example 15A. The last-eluting diastereomer (Diastereomer 2) is described under Example 16A. The absolute stereochemistry of the diastereomers was clarified by X-ray structural analysis.

### Example 15A

### N-{(1S)-2-Amino-2-oxo-1-[3-(trifluoromethyl)phenyl]ethyl}-2-{3-(4-chlorophenyl)-5-oxo-4-[(2S)-3,3,3-trifluoro-2-hydroxypropyl]-4,5-dihydro-1H-1,2,4-triazol-1-yl}acetamide (Diastereomer 1)

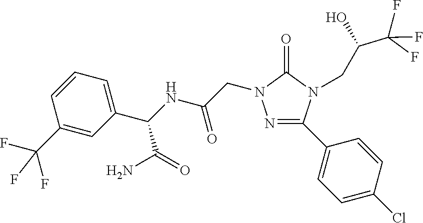

First-eluting diastereomer from the chromatographic resolution (Method 11a) of N-2-amino-2-oxo-1-[3-(trifluoromethyl)phenyl]ethyl}-2-{3-(4-chlorophenyl)-5-oxo-4-[(2S)-3,3,3-trifluoro-2-hydroxypropyl]-4,5-dihydro-1H-1,2,4-triazol-1-yl-acetamide. The absolute configuration was determined by X-ray structural analysis.

Chiral analytical HPLC [Method 12a]: Rₜ=2.65 min.

LC-MS [Method 5]: Rₜ=1.03 min; MS [ESpos]: m/z=566 (M+H)⁺

¹H-NMR (500 MHz, DMSO-d₆): δ [ppm]=3.82 (dd, 1H), 3.96 (dd, 1H), 4.20-4.34 (m, 1H), 4.54-4.65 (m, 2H), 5.51 (d, 1H), 6.90 (d, 1H), 7.33 (s, 1H), 7.58-7.64 (m, 3H), 7.67 (d, 1H), 7.71-7.77 (m, 3H), 7.81 (s, 1H), 7.88 (br. s., 1H), 8.99 (d, 1H).

### Example 16A

### N-{(1R)-2-Amino-2-oxo-1-[3-(trifluoromethyl)phenyl]ethyl}-2-{3-(4-chlorophenyl)-5-oxo-4-[(2S)-3,3,3-trifluoro-2-hydroxypropyl]-4,5-dihydro-1H-1,2,4-triazol-1-yl}acetamide (Diastereomer 2)

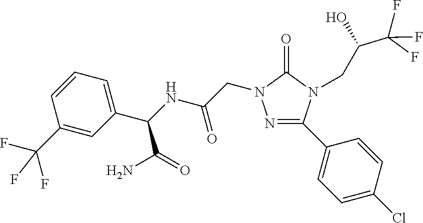

Last-eluting diastereomer from the chromatographic resolution (Method 11a) of N-2-Amino-2-oxo-1-[3-(trifluormethyl)phenyl]ethyl}-2-{3-(4-chlorphenyl)-5-oxo-4-[(2S)-3,3,3-trifluoro-2-hydroxypropyl]-4,5-dihydro-1H-1,2,4-triazol-1-yl-acetamide.

Chiral analytical HPLC [Method 12a]: Rₜ=3.69 min.

LC-MS [Method 5]: Rₜ=1.03 min; MS [ESpos]: m/z=566 (M+H)⁺

¹H-NMR (500 MHz, DMSO-d₆): δ [ppm] 3.82 (dd, 1H), 3.96 (dd, 1H), 4.21-4.32 (m, 1H), 4.53-4.67 (m, 1H), 5.51 (d, 1H), 6.89 (d, 1H), 7.33 (br. s., 1H), 7.58-7.64 (m, 3H), 7.65-7.68 (m, 1H), 7.71-7.76 (m, 3H), 7.81 (s, 1H), 7.88 (br s, 1H), 8.99 (d, 1H).

### Example 17A

### 2-{3-(4-Chlorophenyl)-5-oxo-4-[(2S)-3,3,3-trifluoro-2-hydroxypropyl]-4,5-dihydro-1H-1,2,4-triazol-1-yl}-N-{(S)-cyano-[3-(trifluoromethyl)phenyl]methyl}acetamide

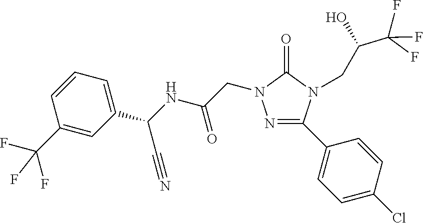

A solution of 400 mg (0.71 mmol) of the compound from Example 15A (S,S-diasteromer) in 8 ml of anhydrous THF was admixed at RT with 126 μl of pyridine (1.56 mmol), then 210 μl of trifluoroacetic anhydride (1.48 mmol). The reaction mixture was concentrated on a rotary evaporator. The residue is purified by preparative HPLC (Method 10). This gave 400 mg of the title compound.

LC-MS [Method 3]: Rₜ=1.32 min; MS [ESpos]: m/z=548 (M+H)⁺

¹H-NMR (400 MHz, DMSO-d₆): d [ppm]=3.84 (dd, 1H), 3.94-4.01 (m, 1H), 4.23-4.37 (m, 1H), 4.61 (q, 2H), 6.37 (d, 1H), 6.93 (d, 1H), 7.64 (d, 2H), 7.70-7.79 (m, 3H), 7.80-7.87 (m, 3H), 9.60 (d, 1H).

### Example 18A

### 2-{3-(4-Chlorophenyl)-5-oxo-4-[(2S)-3,3,3-trifluoro-2-hydroxypropyl]-4,5-dihydro-1H-1,2,4-triazol-1-yl}-N-{(R)-cyano-[3-(trifluoromethyl)phenyl]methyl}acetamide

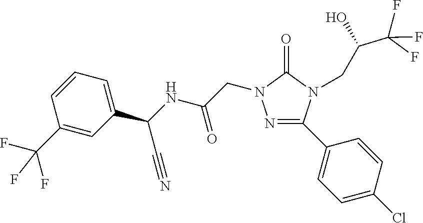

From 340 mg (0.60 mmol) of the compound from Example 16A, in analogy to Example 17A, 268 mg (81% of theory) of the title compound were obtained.

LC-MS [Method 1]: Rₜ=2.16 min; MS [ESpos]: m/z=548 (M+H)⁺

¹H-NMR (400 MHz, DMSO-d₆): δ [ppm]=3.84 (dd, 1H), 3.98 (dd, 1H), 4.22-4.35 (m, 1H), 4.61 (s, 2H), 6.37 (d, 1H), 6.93 (d, 1H), 7.64 (d, 2H), 7.69-7.81 (m, 3H), 7.79-7.87 (m, 3H), 9.61 (d, 1H).

### Example 19A

### tert-Butyl {(phenylsulphonyl)[3-(trifluoromethyl)phenyl]methyl}-carbamate

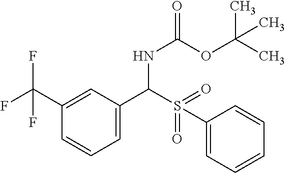

A quantity of 4.49 g (38.29 mmol) of tert-butyl carbamate and 12.57 g (76.57 mmol) of sodium benzenesulphinate were introduced in 110 ml of methanol/water 1:2 and admixed in succession with 10 g (57.43 mmol) of 3-(trifluoromethyl)benzenecarbaldehyde and 2.87 ml (76.09 mmol) of formic acid. The mixture was stirred at RT for 30 h. The precipitated product was isolated by filtration, washed in succession with water and diethyl ether and sucked dry. Further drying in an HV gave 11.2 g (47% of theory) of the title compound.

¹H-NMR (400 MHz, DMSO-d₆): □□ δ [ppm]=8.86 (d, 1H), 8.14 (s, 1H), 7.99 (d, 1H), 7.88 (d, 2H), 7.80 (d, 1H), 7.71-7.77 (m, 1H), 7.59-7.70 (m, 3H), 6.25 (d, 1H), 1.18 (s, 9H).

### Example 20A

### tert-Butyl {(E)-[3-(trifluoromethyl)phenyl]methylidene}carbamate

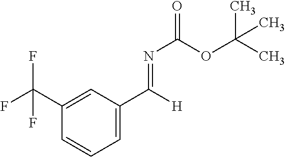

A quantity of 10.88 g (78.73 mmol) of potassium carbonate was dried hot in an HV, and, after cooling, was admixed with 127 ml of THF and also with 5.45 g (13.12 mmol) of the sulphonyl compound from Example 19A. The mixture was heated to reflux under argon for 16 hours. The mixture was cooled to RT, filtered through Celite and washed with THF. The filtrate was concentrated on a rotary evaporator and then dried in an HV. The residue corresponded to the title compound: 3.63 g (100% of theory).

MS [Method 7]: m/z=274 (M+H)⁺

¹H-NMR (400 MHz, DMSO-d₆): δ [ppm]=8.95 (s, 1H), 8.26 (s, 1H), 8.23 (d, 1H), 8.01 (d, 1H), 7.80 (t, 1H), 1.52 (s, 9H).

### Example 21A

### tert-Butyl {1,3-oxazol-2-yl[3-(trifluoromethyl)phenyl]methyl}carbamate (Racemate)

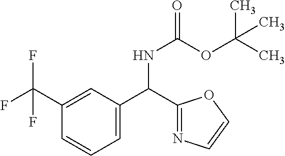

A solution, cooled to −78° C., of 222 mg (3.22 mmol) of oxazole in 20 ml of THF was admixed slowly dropwise with 2.20 ml of an n-butyllithium solution (1.6M in hexane, 3.51 mmol). Following the addition, the colourless solution was stirred at −78° C. for a further 30 minutes, then admixed dropwise with a solution of 800 mg (2.93 mmol) of the compound from Example 20A in 10 ml of THF. The mixture was stirred at −78° C. for a further 30 minutes, then warmed to RT without a cooling bath. After 30 minutes, it was again cooled to −20° C. and the reaction was halted by addition of 5 ml of 10% strength aqueous ammonium chloride solution. A quantity of 150 ml of water was added, and the mixture was extracted twice with ethyl acetate. The combined organic phases were washed with water and with saturated aqueous sodium chloride solution, then dried over sodium sulphate. The solvent was removed on a rotary evaporator and the residue was purified by preparative HPLC (Method 10). This gave 382 mg (35% of theory) of the title compound as a colourless oil.

LC-MS [Method 4]: Rₜ=1.10 min; MS [ESneg]: m/z=341 (M−H)⁻

¹H-NMR (400 MHz, DMSO-d₆): δ [ppm]=1.39 (br. s., 9H), 5.84 (d, 1H), 7.55-7.61 (m, 1H), 7.62-7.69 (m, 2H), 7.73 (s, 1H), 7.84 (s, 1H), 7.98 (d, 1H), 8.32 (s, 1H).

### Example 22A

### 1-(1,3-Oxazol-2-yl)-1-[3-(trifluoromethyl)phenyl]methanamine hydrochloride (Racemate)

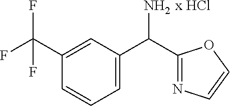

A quantity of 345 mg (1.01 mmol) of the compound from Example 21A in 8 ml of dichloromethane was admixed with 8 ml of a 4N solution of hydrogen chloride in dioxane and stirred at RT for 2 hours. The volatile components were removed on a rotary evaporator and the residue was dried under a high vacuum. This gave 281 mg (100% of theory) of the title compound.

LC-MS [Method 5]: Rₜ=0.54 min; MS [ESpos]: m/z=243 (M+H)⁺

¹H-NMR (400 MHz, DMSO-d₆): δ [ppm]=5.83 (br. s., 1H), 7.71 (t, 1H), 7.80 (d, 1H), 7.87 (d, 1H), 8.01 (s, 1H), 8.15 (s, 1H), 8.56 (s, 1H), 9.07 (br. s., 3H).

### Example 23A

### tert-Butyl {2-hydrazino-2-oxo-1-[3-(trifluoromethyl)phenyl]ethyl}carbamate (Racemate)

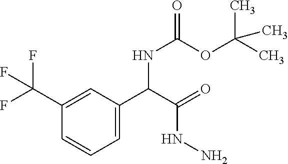

A solution of 640 mg of (DL)-[(tert-butoxycarbonyl)amino][3-(trifluoromethyl)phenyl]acetic acid (2.0 mmol) in 4 ml of acetonitrile was admixed with 500 mg (2.61 mmol) of EDC and 352 mg (2.61 mmol) of HOBt and stirred at RT for 20 minutes. The resulting solution was added dropwise to a solution, cooled to 0° C. beforehand, of hydrazine hydrate (195 μl, 4.01 mmol) and cyclohexene (40.6 mg, 0.49 mmol) in 2 ml of acetonitrile. The entire reaction mixture was stirred for 30 minutes and then admixed with 50 ml of water. The acetonitrile was removed on a rotary evaporator. The aqueous phase which remained was extracted three time with ethyl acetate. The combined organic phases were washed with 2M aqueous sodium carbonate solution, saturated aqueous sodium chloride solution and dried over sodium sulphate. The solvent was removed on a rotary evaporator and the residue was dried under a high vacuum. This gave 652 mg (98% of theory) of the title compound.

LC-MS [Method 2]: Rₜ=1.90 min; MS [ESpos]: m/z=334 (M+H)⁺

¹H-NMR (400 MHz, DMSO-d₆): δ [ppm]=1.37 (s, 9H), 4.25-4.33 (m, 2H), 5.23 (d, 1H), 7.53-7.67 (m, 3H), 7.71 (d, 1H), 7.80 (s, 1H), 9.41-9.47 (m, 1H).

### Example 24A

### tert-Butyl {(5-amino-1,3,4-oxadiazol-2-yl)[3-(trifluoromethyl)phenyl]methyl}carbamate (Racemate)

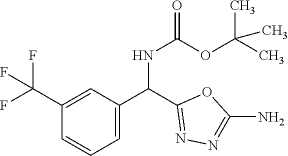

A solution of 300 mg (0.9 mmol) of the compound from Example 23A and 95 mg (0.9 mmol) of cyanogen bromide in 8 ml of methanol was stirred at 60° C. overnight. When an LC-MS check had indicated incomplete reaction, a further 32 mg of cyanogen bromide were added and the mixture was heated to 60° C. for a further 4 hours. After cooling, the volatile constituents were removed on a rotary evaporator. The residue was dissolved in a little DMSO and purified by preparative HPLC (Method 10). The title compound was dried under a high vacuum: 107 mg (33% of theory).

LC-MS [Method 6]: Rₜ=2.06 min; MS [ESpos]: m/z=359 (M+H)⁺

¹H-NMR (400 MHz, DMSO-d₆): δ [ppm]=1.39 (s, 9H), 6.03 (d, 1H), 7.06 (s, 2H), 7.59-7.65 (m, 1H), 7.68-7.76 (m, 2H), 7.83 (s, 1H), 8.27 (d, 1H).

### Example 25A

### 5-{Amino[3-(trifluoromethyl)phenyl]methyl}-1,3,4-oxadiazol-2-amine hydrochloride (Racemate)

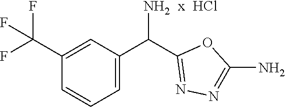

A quantity of 107 mg (0.30 mmol) of the compound from Example 24A was stirred in 5 ml of dichloromethane and 5 ml of a 4N solution of hydrogen chloride in dioxane at RT overnight. The volatile components were removed on a rotary evaporator. The residue was dried under a high vacuum. It corresponded to the title compound (99 mg, 100% of theory).

LC-MS [Method 2]: Rₜ=0.95 min; MS [ESpos]: m/z=259 (M+H)⁺

¹H-NMR (400 MHz, DMSO-d₆): δ [ppm]=6.07 (br. s., 1H), 7.33 (br. s., 2H), 7.71-7.78 (m, 1H), 7.82-7.89 (m, 2H), 8.00 (s, 1H), 9.44 (br. s., 3H).

### Example 26A

### tert-Butyl {(3-methyl-1,2,4-oxadiazol-5-yl)[3-(trifluoromethyl)phenyl]methyl}carbamate (Racemate)

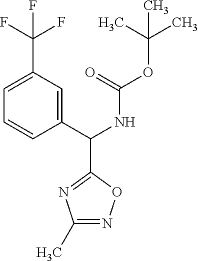

A quantity of 319 mg of (DL)-[(tert-butoxycarbonyl)amino][3-(trifluoromethyl)phenyl]acetic acid (1.0 mmol) in 2 ml of DMF and 6 ml of dichloromethane was admixed with 162 mg (1.2 mmol) of HOBt, 230 mg (1.2 mmol) of EDC, 89 mg (1.2 mmol) of N-hydroxyacetamidine and 261 μl of N,N-diisopropylethylamine and the reaction mixture was stirred at RT overnight. The dichloromethane was removed on a rotary evaporator and the remaining mixture was diluted with ethyl acetate. This organic phase was washed with saturated aqueous sodium hydrogen carbonate solution and then with saturated aqueous sodium chloride solution, dried over sodium sulphate and concentrated on a rotary evaporator to remove the solvent. The residue was heated to reflux in 4 ml of pyridine for 30 minutes, then cooled to RT. The pyridine was removed on a rotary evaporator and the residue was purified by preparative HPLC (Method 10). This gave 237 mg (66% of theory) of the title compound.

LC-MS [Method 1]: Rₜ=0.95 min; MS [ESneg]: m/z=356 (M−H)⁻

¹H-NMR (400 MHz, DMSO-d₆): δ [ppm]=1.40 (s, 9H), 2.26-2.35 (m, 3H), 6.29 (d, 1H), 7.60-7.67 (m, 1H), 7.76 (dd, 2H), 7.89 (s, 1H), 8.43 (d, 1H).

### Example 27A

### 1-(3-Methyl-1,2,4-oxadiazol-5-yl)-1-[3-(trifluoromethyl)phenyl]methanamine hydrochloride (Racemate)

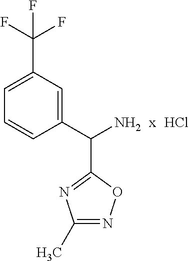

A quantity of 200 mg (0.56 mmol) of the compound from Example 26A was stirred in 6 ml of dichloromethane and 6 ml of a 4N solution of hydrogen chloride in dioxane at RT overnight. The volatile components were removed on a rotary evaporator. The residue was dried under a high vacuum. It corresponded to the title compound (165 mg, 100% of theory).

LC-MS [Method 1]: Rₜ=0.95 min; MS [ESpos]: m/z=258 (M+H)⁺

¹H-NMR (400 MHz, DMSO-d₆): δ [ppm]=2.42 (s, 3H), 6.37 (s, 1H), 7.72-7.79 (m, 1H), 7.88 (d, 2H), 8.06 (s, 1H), 9.58 (br. s., 3H).

### Example 28A

### tert-Butyl {(6-methoxypyridin-2-yl)[3-(trifluoromethyl)phenyl]methyl}carbamate (Racemate)

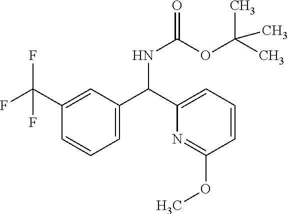

A solution of 344 mg (1.83 mmol) of 2-bromo-6-methoxypyridine in 15 ml of THF at −78° C. was admixed slowly with 1.26 ml of n-butyllithium (solution, 1.6M in hexane, 2.01 mmol). The orange solution was stirred at −78° C. for 30 minutes, then admixed dropwise with a solution of 500 mg (1.83 mmol) of the compound from Example 20A in 5 ml of THF. Following the addition, the mixture was stirred at −78° C. for a further 30 minutes, then warmed slowly to RT. After 30 minutes, it was cooled to −20° C. again, in order to halt the reaction by addition of 5 ml of 10% strength aqueous ammonium chloride solution. The mixture was diluted with 100 ml of water and 20 ml of 2M aqueous sodium carbonate solution, then extracted twice with ethyl acetate. The combined organic phases were washed with water and then with saturated aqueous sodium chloride solution, dried over sodium sulphate and concentrated on a rotary evaporator. The residue was purified by preparative HPLC (Method 10). This gave 268 mg (33% of theory) of the title compound (purity 85% (LC-MS)).

LC-MS [Method 3]: Rₜ=1.52 min; MS [ESpos]: m/z=383 (M+H)⁺

¹H-NMR (400 MHz, DMSO-d₆): δ [ppm]=1.40 (br. s., 9H), 3.81 (s, 3H), 5.88 (d, 1H), 6.67 (d, 1H), 7.06 (d, 1H), 7.51-7.63 (m, 2H), 7.64-7.74 (m, 2H), 7.84 (s, 1H), 7.98 (d, 1H).

### Example 29A

### 1-(6-Methoxypyridin-2-yl)-1-[3-(trifluoromethyl)phenyl]methanamine hydrochloride (Racemate)

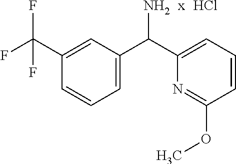

A quantity of 268 mg (0.60 mmol) of the compound from Example 28A was stirred in 4.25 ml of dichloromethane and 4.25 ml of a 4N solution of hydrogen chloride in dioxane at RT for 1 hour.

The volatile components were removed on a rotary evaporator. The residue was dried under a high vacuum. It corresponded to the title compound (237 mg, 85% pure according to LC-MS).

LC-MS [Method 2]: Rₜ=1.50 min; MS [ESpos]: m/z=266 (M+H—NH₂)⁺

¹H-NMR (400 MHz, DMSO-d₆): δ [ppm]=3.98 (s, 3H), 5.79-5.86 (m, 1H), 6.83 (d, 1H), 7.07 (d, 1H), 7.66-7.72 (m, 1H), 7.73-7.79 (m, 2H), 7.89 (d, 1H), 8.09 (s, 1H), 8.99 (br. s., 3H).

### Example 30A

### tert-Butyl {[3-(trifluoromethyl)phenyl](1-trityl-1H-imidazol-4-yl)methyl}carbamate (Racemate)

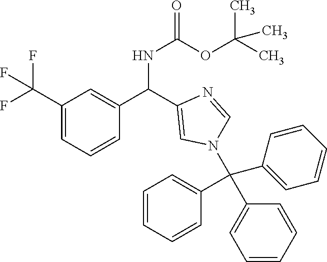

A solution of 319 mg (0.73 mmol) of 4-iodo-1-trityl-1H-imidazole in 7 ml of dichloromethane was admixed at RT with 122 μl of a 3M solution of ethylmagnesium bromide in diethyl ether (0.37 mmol). After 30 minutes, 100 mg (0.37 mmol) of the compound from Example 20A were added. The reaction mixture was left with stirring overnight, then admixed with 1 ml of a 10% strength aqueous ammonium chloride solution and 20 ml of methanol. The insoluble constituents were removed by filtration and the filtrate was concentrated on a rotary evaporator. The residue was separated by preparative HPLC (Method 10). This gave the title compound (57 mg, 27% of theory).

LC-MS [Method 5]: Rₜ=1.45 min; MS [ESpos]: m/z=584 (M+H)⁺

¹H-NMR (400 MHz, DMSO-d₆): δ [ppm]=1.35 (br s, 9H), 5.74 (br d, 1H), 6.84 (br s, 1H), 7.04-7.10 (m, 6H), 7.29 (d, 1H), 7.36-7.43 (m, 9H), 7.49-7.61 (m, 4H), 7.75 (d, 1H).

### Example 31A

### 1-(1H-Imidazol-4-yl)-1-[3-(trifluoromethyl)phenyl]methanamine dihydrochloride (Racemate)

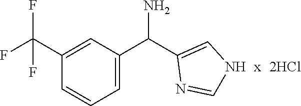

A quantity of 57 mg (98 μmol) of the compound from Example 30A was stirred in 2 ml of a 4N solution of hydrogen chloride in dioxane at RT overnight. The volatile components were then removed on a rotary evaporator under a high vacuum. The residue was stirred with diethyl ether. The solid was isolated by filtration, washed with a little ether and dried under a high vacuum, and corresponded to the title compound (29 mg, 95% of theory)

LC-MS [Method 5]: Rₜ=0.33 min; MS [ESneg]: m/z=240 (M−H)⁻

¹H-NMR (400 MHz, DMSO-d₆): δ [ppm]=5.94 (br. s., 1H), 7.62-7.74 (m, 2H), 7.81 (d, 1H), 7.91 (d, 1H), 8.05 (s, 1H), 8.79 (br. s., 1H), 9.41 (br. s., 3H), ca. 14.1 ppm (very broad, 2H).

### Example 32A

### N-{(2Z)-2-Amino-2-(hydroxyimino)-1-[3-(trifluoromethyl)phenyl]ethyl}-2-{3-(4-chlorophenyl)-5-oxo-4-[(2S)-3,3,3-trifluoro-2-hydroxypropyl]-4,5-dihydro-1H-1,2,4-triazol-1-yl}acetamide (Diastereomer Mixture)

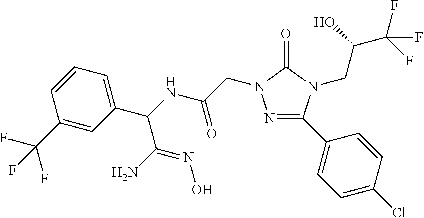

A quantity of 634 mg (9.1 mmol) of hydroxylamine hydrochloride was dissolved in 25 ml of DMSO and admixed with stirring with 1.27 ml (9.1 mmol) of triethylamine. After 10 minutes, the resulting precipitate was removed by filtration and the filtrate was admixed with 1.00 g (1.83 mmol) of the compound from Example 17A. The reaction mixture was heated to 75° C. for 2 hours, then left to cool to RT and diluted with ethyl acetate. The organic phase was then washed three times with water and once with saturated aqueous sodium chloride solution. The organic phase was dried over sodium sulphate and freed from the solvent on a rotary evaporator. The residue was dried under a high vacuum. This gave 1.02 g (79% of theory) of the title compound in a purity of approximately 82% (LC-MS).

LC-MS [Method 2]: Rₜ=2.11 min; MS [ESpos]: m/z=581 (M+H)⁺

¹H-NMR (400 MHz, DMSO-d₆): δ [ppm]=3.77-3.88 (dd, 1H), 3.92-4.06 (br d, 1H), 4.22-4.34 (m, 1H), 4.56-4.60 (m, 2H), 5.57 (d, 1H), 5.65 (br. s., 2H), 6.88-6.94 (m (2 d, 1 d each per diastereomer), 1H), 7.54-7.70 (m, 5H), 7.70-7.77 (m, 3H), 8.89 (d, 1H), 9.30 (br. s., 1H).

### Example 33A

### Methyl 3-[({3-(4-chlorophenyl)-5-oxo-4-[(2S)-3,3,3-trifluoro-2-hydroxypropyl]-4,5-dihydro-1H-1,2,4-triazol-1-yl}acetyl)amino]-3-(2-fluorophenyl)propionate (diastereomer mixture)

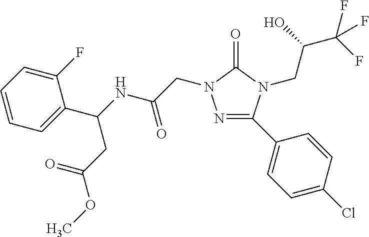

Of the compound from Example 8A, 50 mg (0.14 mmol) were dissolved in 1 ml of DMF, admixed with 34 mg (0.18 mmol) of EDC and with 22 mg (0.16 mmol) of HOBt, and stirred at room temperature for 10 minutes. Then 35 mg (0.15 mmol) of methyl 3-amino-3-(2-fluorophenyl)propionate hydrochloride and also 20 μl (0.15 mmol) of triethylamine were added and the mixture was left with stirring at room temperature for 16 h. For work-up, it was admixed with 10 ml of water and extracted with twice 10 ml of ethyl acetate. The combined organic phases were dried over magnesium sulphate, filtered and concentrated on a rotary evaporator. The crude product was purified by preparative HPLC [Method 13]. This gave 47 mg (63% of theory) of the target compound.

LC-MS [Method 3] Rₜ=1.22 min; MS [ESIpos]: m/z=545 (M+H)⁺

¹H-NMR (400 MHz, CDCl₃): δ [ppm]=□□ 2.80-2.96 (m, 2H), 3.53 and 3.58 (2s, 3H), 3.93-4.12 (m, 2H), 4.44-4.82 (m, 3H), 5.05 (t, 1H), 5.56-5.67 (m, 1H), 6.98-7.24 (m, 3H), 7.27-7.37 (m, 2H), 7.47-7.64 (m, 3H), 7.70 (d, 2H). (Partial resolution of the duplicated signal set of the diastereomer mixture.)

### Example 34A

### Methyl {3-(4-chlorophenyl)-5-oxo-4-[(1E)-3,3,3-trifluoroprop-1-en-1-yl]-4,5-dihydro-1H-1,2,4-triazol-1-yl}acetate

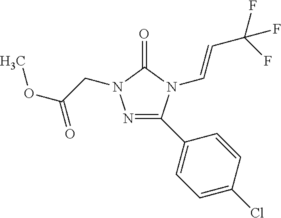

A quantity of 280 mg (0.74 mmol) of the compound from Example 7A was introduced at RT together with 108.1 mg (0.89 mmol) of 4-dimethylaminopyridine in 5.3 ml of pyridine, admixed in portions with 0.31 ml of trifluoromethanesulphonic anhydride (1.84 mmol) and stirred for 12 hours. The pyridine was removed on a rotary evaporator and the residue was taken up in acetonitrile and 1N hydrochloric acid. It was purified by preparative HPLC (Method 10). This gave 230 mg (86% of theory) of the clean title compound.

LC/MS [Method 4]: Rₜ=1.14 min; m/z=362 (M+H)⁺

¹H-NMR (400 MHz, DMSO-d₆): δ [ppm]=□□ 7.68 (s, 4H), 7.18 (d, 1H), 6.85 (dd, 1H), 4.78 (s, 2H), 3.72 (s, 3H).

### Example 35A

### {3-(4-Chlorophenyl)-5-oxo-4-[(1E)-3,3,3-trifluoroprop-1-en-1-yl]-4,5-dihydro-1H-1,2,4-triazol-1-yl}acetic acid

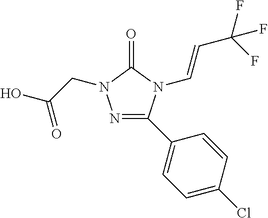

A quantity of 260 mg (0.72 mmol) of the compound from Example 34A was dissolved in 5 ml of methanol and admixed with 2.87 ml (2.87 mmol) of a 1 M solution of lithium hydroxide in water. The mixture was stirred at RT for 1 hour, then acidified with 1N hydrochloric acid and diluted with DMSO. The entire solution was purified by preparative HPLC (Method 10). This gave 215 mg (86% of theory) of the clean title compound.

LC/MS [Method 4]: Rₜ=1.03 min; m/z=348 (M+H)⁺

¹H-NMR (400 MHz, DMSO-d₆): δ [ppm]=□□ 13.31 (br. s, 1H), 7.68 (s, 4H), 7.19 (dd, 1H), 6.79-6.92 (m, 1H), 4.64 (s, 2H).

### Example 36A

### [3-(4-Chlorophenyl)-5-oxo-4-(3,3,3-trifluoropropyl)-4,5-dihydro-1H-1,2,4-triazol-1-yl]acetic acid

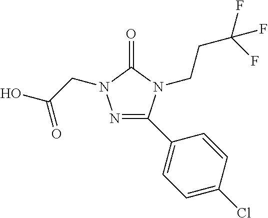

A quantity of 1.2 g (3.45 mmol) of the compound from Example 35A was hydrogenated with 150 mg of platinum on carbon (5%) in 100 ml of methanol under atmospheric pressure overnight. The catalyst was removed by filtration and the solvent was removed on a rotary evaporator. The crude product was purified by preparative HPLC (Method 15). The appropriate fraction was freed from the solvent on the rotary evaporator. The residue was dried in an HV: this gave 945 mg (78% of theory) of the clean title compound.

LC/MS [Method 4]: Rₜ=0.88 min; m/z=350 (M+H)⁺.

¹H-NMR (400 MHz, DMSO-d₆): δ [ppm]=13.14 (br. s., 1H), 7.62-7.72 (m, 4H), 4.56 (s, 2H), 4.01 (t, 2H), 2.54-2.68 (m, 2H).

### Implementing Examples

### Example 1

### 2-{3-(4-Chlorophenyl)-5-oxo-4-[(2S)-3,3,3-trifluoro-2-hydroxypropyl]-4,5-dihydro-1H-1,2,4-triazol-1-yl}-N-{(1R)-1-(5-oxo-4,5-dihydro-1,2,4-oxadiazol-3-yl)-1-[3-(trifluoro-methyl)phenyl]ethyl}acetamide

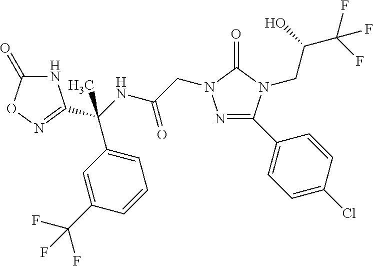

A quantity of 90 mg of the compound from Example 13A (61 mmol) was introduced in 1 ml of DMF and admixed with 5 μl of pyridine (67 mmol). The reaction solution was cooled to 0° C., admixed slowly with 8 μl (8.3 mg, 61 mmol) of isobutyl chloroformate and stirred for 40 minutes thereafter. The reaction mixture was admixed with water and extracted three times with ethyl acetate. The combined organic phases were washed with saturated aqueous sodium chloride solution, dried over sodium sulphate and freed from the solvent on a rotary evaporator. The residue was dried under a high vacuum. This gave 30 mg (40 mmol) of crude N-{(2R)-1-amino-1-{[(isobutoxycarbonyl)oxy]imino}-2-[3-(trifluoromethyl)phenyl]propan-2-yl}-2-{3-(4-chloro-phenyl)-5-oxo-4-[(2S)-3,3,3-trifluoro-2-hydroxypropyl]-4,5-dihydro-1H-1,2,4-triazol-1-yl}acetamide. This intermediate was admixed with 1 ml of DMF and 17.4 mg (182 mmol) of sodium tert-butylate and stirred at RT overnight. The reaction mixture was admixed with 1 ml of 1M hydrochloric acid. The entire solution was separated by preparative HPLC (method 10). This gave 24 mg (64% of theory) of the title compound.

LC-MS [Method 6]: Rₜ=2.46 min; MS [ESpos]: m/z=621 (M+H)⁺

¹H-NMR (400 MHz, DMSO-d₆): δ [ppm]=1.93 (s, 3H), 3.83 (dd, 1H), 3.98 (dd, 1H), 4.22-4.33 (m, 1H), 4.57-4.68 (m, 2H), 6.91 (d, 1H), 7.61-7.67 (m, 3H), 7.72-7.82 (m, 4H), 7.84 (br s, 1H), 9.18 (s, 1H), 12.62 (s, 1H).

### Example 2

### N-{(1R)-1-(5-Amino-1,3,4-oxadiazol-2-yl)-1-[3-(trifluoromethyl)phenyl]ethyl}-2-{3-(4-chlorophenyl)-5-oxo-4-[(2S)-3,3,3-trifluoro-2-hydroxypropyl]-4,5-dihydro-1H-1,2,4-triazol-1-yl}acetamide

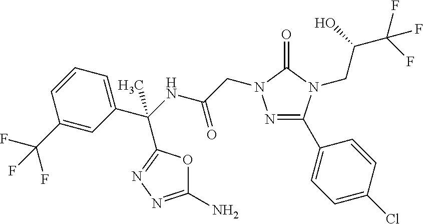

A quantity of 100 mg (0.168 mmol) of the compound from Example 14A and 17.8 mg (0.168 mmol) of cyanogen bromide were combined in 1.5 ml of methanol and stirred at 60° C. for 4 hours. Then a further 5.3 mg (0.050 mmol) of cyanogen bromide were added and stirring at 60° C. was continued for 1 hour. The reaction mixture was diluted with a little acetonitrile, DMSO and 1 ml of 1M hydrochloric acid and separated in its entirety by preparative HPLC (Method 10). This gave 60 mg (58% of theory) of the title compound.

LC-MS [Method 3]: Rₜ=1.19 min; MS [ESpos]: m/z=620 (M+H)⁺

¹H-NMR (400 MHz, DMSO-d₆): δ [ppm]=2.01 (s, 3H), 3.82 (dd, 1H), 3.96 (dd, 1H), 4.21-4.33 (m, 1H), 4.52-4.63 (m, 2H), 6.91 (d, 1H), 7.03 (s, 2H), 7.59-7.66 (m, 3H), 7.66-7.78 (m, 5H), 9.11 (s, 1H).

### Example 3

### 2-{3-(4-Chlorophenyl)-5-oxo-4-[(2S)-3,3,3-trifluoro-2-hydroxypropyl]-4,5-dihydro-1H-1,2,4-triazol-1-yl}-N-{(1R)-1-(3-methyl-1,2,4-oxadiazol-5-yl)-1-[3-(trifluoro-methyl)phenyl]ethyl}acetamide

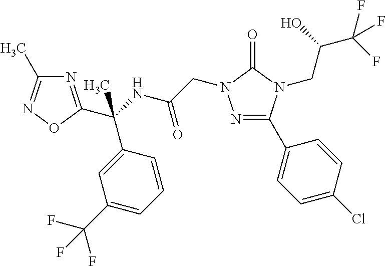

A quantity of 150 mg (0.26 mmol) of the compound from Example 10A, 51 mg (0.36 mmol) of HOBt, 69 mg (0.36 mmol) of EDC, 27 mg of N-hydroxyacetamidine und 90 μl (0.51 mmol) of N,N-diisopropylethylamine were stirred in 3 ml of DMF und 2.7 ml of dichloromethane at RT overnight. The dichloromethane was then removed on a rotary evaporator and the remainder of the mixture was diluted with ethyl acetate. The organic phase was washed with saturated aqueous sodium hydrogen carbonate solution and saturated aqueous sodium chloride solution, dried over sodium sulphate and the solvent was removed on a rotary evaporator. The residue was taken up in 4 ml of pyridine and the solution was heated to reflux for 30 minutes. After cooling to RT, the volatile components were removed on a rotary evaporator. The residue was dissolved in a little DMSO/acetonitrile and purified by preparative HPLC (Method 10). This gave 96 mg (60% of theory) of the title compound.

LC-MS [Method 3]: Rₜ=1.35 min; MS [ESpos]: m/z=619 (M+H)⁺

¹H-NMR (400 MHz, DMSO-d₆): δ [ppm]=2.06 (d, 3H), 2.31 (s, 3H), 3.82 (dd, 1H), 3.96 (dd, 1H), 4.20-4.33 (m, 1H), 4.56-4.71 (m, 2H), 6.90 (d, 1H), 7.61-7.68 (m, 3H), 7.71-7.77 (m, 3H), 7.80 (d, 1H), 7.84 (s, 1H), 9.38 (s, 1H).

### Example 4

### 2-{3-(4-Chlorophenyl)-5-oxo-4-[(2S)-3,3,3-trifluoro-2-hydroxypropyl]-4,5-dihydro-1H-1,2,4-triazol-1-yl}-N-{(1R)-1-(1H-tetrazol-5-yl)-1-[3-(trifluoromethyl)phenyl]ethyl}acetamide

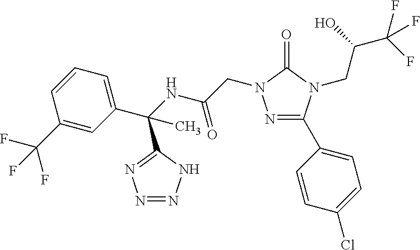

A quantity of 47.0 mg (84 μmol) of the compound from Example 12A was stirred with 2.1 mg (8 μmol) of di-n-butyltin oxide and 19.3 mg (167 μmol) of trimethylsilyl azide in 1 ml of toluene at reflux overnight. After cooling to RT, 2 ml of methanol were added and the mixture was stirred at RT for 1 hour. The solvent was removed on a rotary evaporator and the residue was purified by preparative HPLC (Method 10). This gave 30 mg (59% of theory) of the title compound.

LC-MS [Method 3]: Rₜ=1.24 min; MS [ESpos]: m/z=605 (M+H)⁺

¹H-NMR (400 MHz, DMSO-d₆): δ [ppm]=2.06 (s, 3H), 3.81 (dd, 1H), 3.94 (dd, 1H), 4.20-4.32 (m, 1H), 4.57-4.69 (m, 2H), 6.89 (d, 1H), 7.57-7.65 (m, 3H), 7.66-7.76 (m, 4H), 7.77 (s, 1H), 9.33 (s, 1H), about 16.3 (very broad, 1H).

### Example 5

### 2-{3-(4-Chlorophenyl)-5-oxo-4-[(2S)-3,3,3-trifluoro-2-hydroxypropyl]-4,5-dihydro-1H-1,2,4-triazol-1-yl}-N-{[5-(trifluoromethyl)-1,2,4-oxadiazol-3-yl][3-(trifluoro-methyl)phenyl]methyl}acetamide (diastereomer mixture)

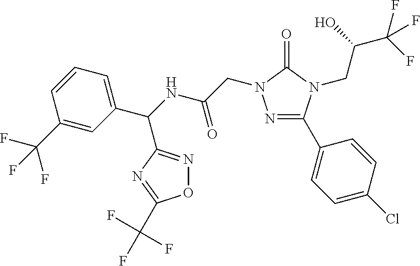

A quantity of 300.0 mg (0.52 mmol) of the compound from Example 32A was introduced in 7 ml of dichloromethane at RT and admixed with 86 μl (0.62 mmol) of triethylamine and 219 μl (1.55 mmol) of trifluoroacetic anhydride and the mixture was stirred at reflux overnight. After cooling, the volatile constituents of the reaction mixture were removed on a rotary evaporator. The residue obtained was purified by preparative HPLC (Method 10). The appropriate fractions were freed from the solvents on a rotary evaporator and the residue was dried under a high vacuum. This gave 275 mg (81% of theory) of the title compound.

LC-MS [Method 3]: Rₜ=1.46 min; MS [ESpos]: m/z=659 (M+H)⁺¹H-NMR (400 MHz, DMSO-d₆): δ [ppm]=3.83 (dd, 1H), 3.97 (dd, 1H), 4.21-4.34 (m, 1H), 4.62 (q, 1H), 4.57-4.66 (m [AB], 1H), 6.60 (d, 1H), 6.91 (d, 1H), 7.60-7.70 (m, 3H), 7.71-7.83 (m, 4H), 7.92 (s, 1H), 9.61 (d, 1H).

### Example 6

### 2-{3-(4-Chlorophenyl)-5-oxo-4-[(2S)-3,3,3-trifluoro-2-hydroxypropyl]-4,5-dihydro-1H-1,2,4-triazol-1-yl}-N-{(5-methyl-1,2,4-oxadiazol-3-yl)[3-(trifluoromethyl)phenyl]methyl}acetamide (Diastereomer mixture)

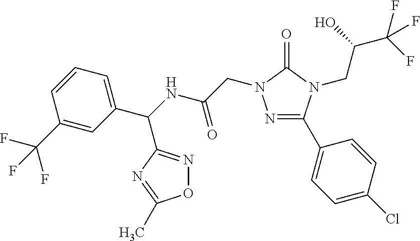

A quantity of 100.0 mg (0.17 mmol) of the compound from Example 32A, 34 mg (0.24 mmol) of HOBt, 46 mg (0.24 mmol) of EDC, 11 μl (0.19 mmol) of acetic acid and 36 μl (0.21 mmol) of N,N-diisopropylethylamine were dissolved in 1 ml of DMF and 4 ml of dichloromethane and stirred at RT for 3 hours. The dichloromethane was removed on a rotary evaporator. The remaining reaction mixture was admixed with 2 ml of pyridine and heated to reflux for 2 hours. The pyridine was then removed on a rotary evaporator. The residue was diluted with 5 ml of DMSO and separated by preparative HPLC (Method 10). The resultant product was purified further by means of preparative thin-layer chromatography (silica gel, eluents: dichloromethane/methanol 100:5), then purified again by preparative HPLC (Method 10). This gave 4 mg (4% of theory) of the title compound.

LC-MS [Method 5]: Rₜ=1.15 min; MS [ESpos]: m/z=605 (M+H)⁺

¹H-NMR (400 MHz, DMSO-d₆): δ [ppm]=2.59 (s, 3H), 3.82 (dd, 1H), 3.93-4.00 (m, 1H), 4.21-4.33 (m, 1H), 4.57-4.62 (m, 2H), 6.38 (d, 1H), 6.89-6.92 (dd, interpreted as 1d per diastereomer, 1H), 7.60-7.66 (m, 3H), 7.70-7.77 (m, 4H), 7.84 (s, 1H), 9.50 (d, 1H).

### Example 7

### 2-{3-(4-Chlorophenyl)-5-oxo-4-[(2S)-3,3,3-trifluoro-2-hydroxypropyl]-4,5-dihydro-1H-1,2,4-triazol-1-yl}-N-{1,2,4-oxadiazol-3-yl[3-(trifluoromethyl)phenyl]methyl}acetamide (Diastereomer mixture)

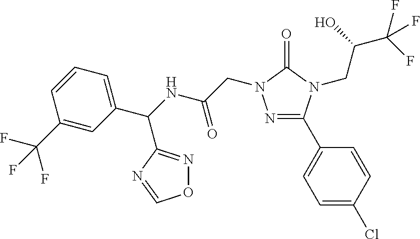

A quantity of 320 mg (0.55 mmol) of the compound from Example 32A were heated together with 2 ml (12.0 mmol) of triethyl orthoformate and 0.5 ml (3.95 mmol) of boron trifluoride-diethyl ether complex to reflux for 30 minutes. The volatile constituents were removed on a rotary evaporator and the residue was taken up in DMSO and purified by preparative HPLC (Method 10). The resultant product was purified further by dissolving it in 1 ml of dichloromethane and purifying it by silica gel chromatography (eluents: cyclohexane/ethyl acetate 2:1). Further purification by preparative HPLC (Method 10) gave 6 mg (2% of theory) of the title compound.

LC-MS [Method 2]: Rₜ=1.10 min; MS [ESpos]: m/z=591 (M+H)⁺

¹H-NMR (400 MHz, DMSO-d₆): δ [ppm]=3.82 (dd, 1H), 3.93-4.00 (m, 1H), 4.22-4.33 (m, 1H), 4.54-4.66 (m, 2H), 6.48 (d, 1H), 6.91 (d, 1H), 7.60-7.67 (m, 3H), 7.71-7.79 (m, 4H), 7.86 (s, 1H), 9.53 (d, 1H), 9.65 (s, 1H).

### Example 8

### 2-{3-(4-Chlorophenyl)-5-oxo-4-[(2S)-3,3,3-trifluoro-2-hydroxypropyl]-4,5-dihydro-1H-1,2,4-triazol-1-yl}-N-{(5-oxo-4,5-dihydro-1,2,4-oxadiazol-3-yl)[3-(trifluoro-methyl)phenyl]methyl}acetamide (Diastereomer mixture)

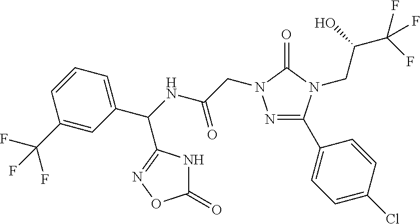

A solution of 194 mg (0.33 mmol) of the compound from Example 32A in 3.5 ml of DMF was admixed with 30 μl (0.37 mmol) of pyridine, then cooled to 0° C. and admixed dropwise with 43 μl of isobutyl chloroformate. The mixture was stirred at RT for 40 minutes. Water was added and the mixture was extracted three times with ethyl acetate. The combined organic phases were washed with saturated aqueous sodium chloride solution, dried over sodium sulphate and concentrated on a rotary evaporator. This gave 200 mg of intermediate. Of this product, 50 mg (73 μmol) were dissolved in 3 ml of DMF and stirred with 21.2 mg (0.22 mmol) of sodium tert-butylate at RT overnight. The reaction mixture was admixed with 1 ml of 1N hydrochloric acid and separated in its entirety by preparative HPLC (Method 10). The appropriate fraction was freed from the solvents on a rotary evaporator and the residue was dried under a high vacuum. This gave 26 mg (58% of theory) of the clean title compound as a diastereomer mixture (according to NMR, two diastereomers in a ratio of 1:1).

LC-MS [Method 3]: Rₜ=1.25 min/1.26 min (double peak); MS [ESpos]: m/z=607 (M+H)⁺

¹H-NMR (400 MHz, DMSO-d₆): δ [ppm]=3.84 (dd, 1H), 3.94-4.01 (“dt” interpreted as 1 dd per diastereomer, 1H), 4.21-4.34 (m, 1H), 4.59 (s, 2H), 4.60 (q, 2H), 6.22 (“dd”, 1 d per diastereomer, 1H), 6.91 (“dd”, 1 d per diastereomer, 1H), 7.60-7.68 (m, 3H), 7.71-7.78 (m, 4H), 7.85 (s, 1H), 9.44 (“t”, interpreted as 1 d per diastereomer, 1H), 12.75 (br. s., 1H).

### Example 9

### 2-{3-(4-Chlorophenyl)-5-oxo-4-[(2S)-3,3,3-trifluoro-2-hydroxypropyl]-4,5-dihydro-1H-1,2,4-triazol-1-yl}-N-{1H-tetrazol-5-yl[3-(trifluoromethyl)phenyl]methyl}acetamide (Diastereomer mixture)

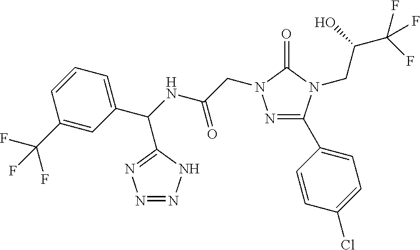

A quantity of 50.0 mg (91 μmol) of the compound from Example 18A, 2.3 mg (9 μmol) of di-n-butyltin oxide and 24 μl (183 μmmol) of trimethylsilyl azide were heated to reflux in 1 ml of toluene overnight. After cooling to RT, 2 ml of methanol were added and the mixture was stirred for 1 hour. The solvents were removed on a rotary evaporator and the residue was purified by preparative HPLC (Method 10). This gave 46 mg (85% of theory) of the title compound as a 3:1 diastereomer mixture.

LC-MS [Method 3]: Rₜ=1.20 min; MS [ESpos]: m/z=591 (M+H)⁺

¹H-NMR (400 MHz, DMSO-d₆): δ [ppm]=3.83 (dd, 1H), 3.93-4.00 (m, 1H), 4.20-4.33 (m, 1H), 4.55-4.67 (m, 2H), 6.61 (d, 1H), 6.89 (d, 0.75H), 6.91 (d, 0.25H), 7.60-7.68 (m, 3H), 7.70-7.76 (m, 4H), 7.83 (s, 1H), 9.53-9.58 (m, 1H, interpreted as 9.55, d, 1H secondary diastereomer and 9.56, d, 1H main diastereomer), 16.11-16.65 (s, 1H).

### Example 10

### 2-{3-(4-Chlorophenyl)-5-oxo-4-[(2S)-3,3,3-trifluoro-2-hydroxypropyl]-4,5-dihydro-1H-1,2,4-triazol-1-yl}-N-{1,3-oxazol-2-yl[3-(trifluoromethyl)phenyl]methyl}acetamide (Diastereomer mixture)

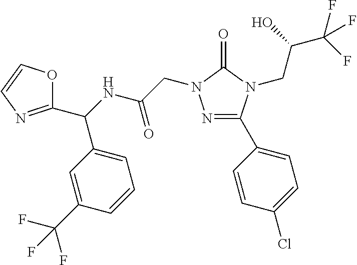

A quantity of 335 mg (0.92 mmol) of the compound from Example 8A, 281.0 mg (1.01 mmol) of the compound from Example 22A, 246 mg (1.28 mmol) of EDC, 173 mg (1.28 mmol) of HOBt and 224 μl (1.28 mmol) of N,N-diisopropylethylamine were stirred in 13 ml of DMF at RT for 2 hours. The reaction solution was then separated by preparative HPLC (Method 10). This gave 522 mg (91% of theory) of the title compound.

LC-MS [Method 4]: Rₜ=1.10 min; MS [ESpos]: m/z=590 (M+H)⁺

¹H-NMR (400 MHz, DMSO-d₆): δ [ppm]=3.83 (dd, 1H), 3.93-4.01 (m, 1H), 4.22-4.34 (m, 1H), 4.58 (s, 2H), 6.20 (d, 1H), 6.90 (dd, 1H (1 d per diastereomer)), 7.56-7.71 (m, 5H), 7.72-7.77 (m, 3H), 8.02 (s, 1H), 8.40 (s, 1H), 9.23 (d, 1H).

Chromatography on a chiral phase (Method 11c) resolved the two diastereomers: see Example 11 and Example 12.

### Example 11

### 2-{3-(4-Chlorophenyl)-5-oxo-4-[(2S)-3,3,3-trifluoro-2-hydroxypropyl]-4,5-dihydro-1H-1,2,4-triazol-1-yl}-N-{1,3-oxazol-2-yl[3-(trifluoromethyl)phenyl]methyl}acetamide (Diastereomer 1)

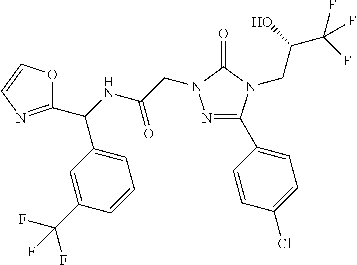

First-eluting diastereomer from the chromatographic diastereomer separation according to Method 11c of 520 mg of the compound from Example 10. The product obtained was also purified by preparative HPLC (Method 10). This gave 215 mg of the title compound.

Analytical chiral HPLC (Method 12a): Rₜ=1.46 min

LC-MS [Method 4]: Rₜ=1.10 min; MS [ESpos]: m/z=590 (M+H)⁺

¹H-NMR (400 MHz, DMSO-d₆): δ [ppm]=3.825 (dd, 1H), 3.97 (dd, 1H), 4.22-4.31 (m, 1H), 4.59 (s, 2H), 6.19 (d, 1H), 6.90 (d, 1H), 7.57-7.71 (m, 5H), 7.72-7.77 (m, 3H), 8.02 (s, 1H), 8.40 (s, 1H), 9.23 (d, 1H).

### Example 12

### 2-{3-(4-Chlorophenyl)-5-oxo-4-[(2S)-3,3,3-trifluoro-2-hydroxypropyl]-4,5-dihydro-1H-1,2,4-triazol-1-yl}-N-{1,3-oxazol-2-yl[3-(trifluoromethyl)phenyl]methyl}acetamide (Diastereomer 2)

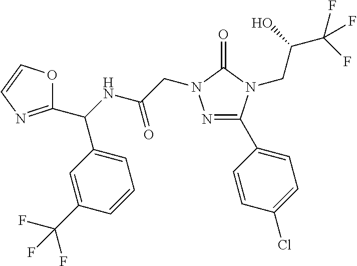

Last-eluting diastereomer from the chromatographic diastereomer separation according to Method 11c of 520 mg of the compound from Example 10. The product obtained was also purified by preparative HPLC (Method 10). This gave 217 mg of the title compound.

Analytical chiral HPLC (Method 12a): Rₜ=1.82 min

LC-MS [Method 5]: Rₜ=1.12 min; MS [ESpos]: m/z=590 (M+H)⁺

¹H-NMR (400 MHz, DMSO-d₆): δ [ppm]=3.825 (dd, 1H), 3.96 (dd, 1H), 4.22-4.33 (m, 1H), 4.58 (s, 2H), 6.20 (d, 1H), 6.91 (d, 1H), 7.56-7.71 (m, 5H), 7.72-7.77 (m, 3H), 8.02 (s, 1H), 8.40 (s, 1H), 9.23 (d, 1H).

### Example 13

### N-{(5-Amino-1,3,4-oxadiazol-2-yl)[3-(trifluoromethyl)phenyl]methyl}-2-{3-(4-chlorophenyl)-5-oxo-4-[(2S)-3,3,3-trifluoro-2-hydroxypropyl]-4,5-dihydro-1H-1,2,4-triazol-1-yl}acetamide (Diastereomer mixture)

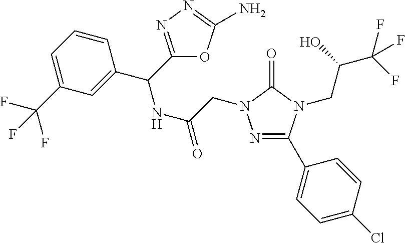

A quantity of 50.0 mg (0.137 mmol) of the compound from Example 8A, 27.7 mg (0.205 mmol) of HOBt, 39.3 mg (0.205 mmol) of EDC, 49.8 mg (0.150 mmol) of the compound from Example 25A and 52 μl (0.301 mmol) of N,N-diisopropylethylamine were dissolved in 1.3 ml of DMF and stirred at RT for 1 hour. This mixture was admixed with 2.0 ml of 1M hydrochloric acid and purified by preparative HPLC (Method 10). This gave 72 mg (87% of theory) of the title compound.

LC-MS [Method 3]: Rₜ=1.17 min; MS [ESpos]: m/z=606 (M+H)⁺ and 1.19 min; MS [ESpos]: m/z=606 (M+H)⁺ (two diastereomers in a ratio of 1:1)

¹H-NMR (400 MHz, DMSO-d₆): δ [ppm]=3.83 (dd, 1H), 3.96 (br d, 1H), 4.23-4.24 (m, 1H), 4.51-4.65 (m, 2H), 6.35 (d, 0.5H), 6.36 (d, 0.5H), 6.93 (br s, 1H), 7.18 (br s, 2H), 7.60-7.67 (m, 3H), 7.71-7.78 (m, 4H), 7.83 (s, 1H), 9.51 (d, 1H).

### Example 14

### 2-{3-(4-Chlorophenyl)-5-oxo-4-[(2S)-3,3,3-trifluoro-2-hydroxypropyl]-4,5-dihydro-1H-1,2,4-triazol-1-yl}-N-{(3-methyl-1,2,4-oxadiazol-5-yl)[3-(trifluoromethyl)phenyl]methyl}acetamide (Diastereomer mixture)

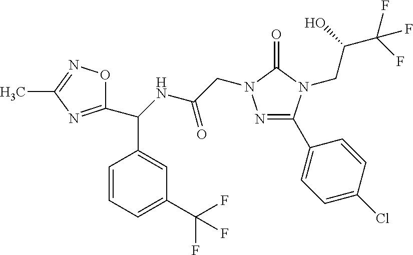

A quantity of 80.0 mg (0.219 mmol) of the compound from Example 8A, 44.3 mg (0.328 mmol) of HOBt, 62.9 mg (0.328 mmol) of EDC, 96.3 mg (0.328 mmol) of the compound from Example 27A and 76 μl (0.438 mmol) of N,N-diisopropylethylamine were dissolved in 2.0 ml of DMF and stirred at RT for 1 hour. This mixture was admixed with 1.0 ml of 1M hydrochloric acid and purified by preparative HPLC (Method 10). This gave 118 mg (89% of theory) of the title compound.

LC-MS [Method 1]: Rₜ=2.19 min; MS [ESpos]: m/z=605 (M+H)⁺

Preparative chromatography on a chiral phase (Method 8) resolved the two diastereomers: see Example 15 and Example 16.

### Example 15

### 2-{3-(4-Chlorophenyl)-5-oxo-4-[(2S)-3,3,3-trifluoro-2-hydroxypropyl]-4,5-dihydro-1H-1,2,4-triazol-1-yl}-N-{(3-methyl-1,2,4-oxadiazol-5-yl)[3-(trifluoromethyl)phenyl]methyl}acetamide (Diastereomer 1)

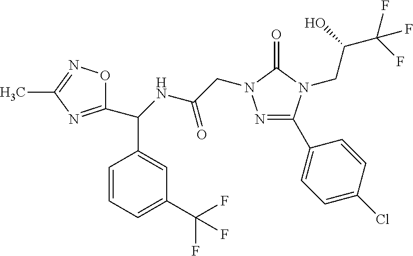

First-eluting diastereomer from the chromatographic diastereomer separation by Method 8 of 118 mg of the compound from Example 14. The product obtained was further purified by preparative HPLC (Method 10). This gave 42 mg of the title compound.

Analytical chiral HPLC (Method 9): Rₜ=2.37 min

LC-MS [Method 2]: Rₜ=2.45 min; MS [ESpos]: m/z=605 (M+H)⁺

¹H-NMR (400 MHz, DMSO-d₆): δ [ppm]=2.34 (s, 3H), 3.83 (dd, 1H), 3.97 (dd, 1H), 4.22-4.33 (m, 1H), 4.56-4.68 (m, 2H), 6.59 (d, 1H), 6.92 (d, 1H), 7.60-7.70 (m, 3H), 7.72-7.81 (m, 4H), 7.90 (s, 1H), 9.63 (d, 1H).

### Example 16

### 2-{3-(4-Chlorophenyl)-5-oxo-4-[(2S)-3,3,3-trifluoro-2-hydroxypropyl]-4,5-dihydro-1H-1,2,4-triazol-1-yl}-N-{(3-methyl-1,2,4-oxadiazol-5-yl)[3-(trifluoromethyl)phenyl]methyl}acetamide (Diastereomer 2)

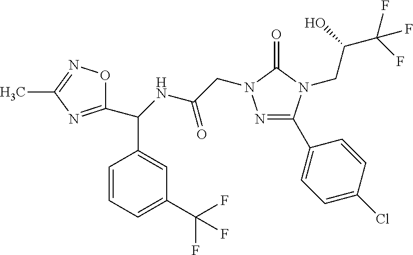

Last-eluting diastereomer from the chromatographic diastereomer separation by Method 8 of 118 mg of the compound from Example 14. The product obtained was further purified by preparative HPLC (Method 10). This gave 42 mg of the title compound.

Analytical chiral HPLC (Method 9): Rₜ=3.25 min

LC-MS [Method 2]: Rₜ=2.44 min; MS [ESpos]: m/z=605 (M+H)⁺

¹H-NMR (400 MHz, DMSO-d₆): δ [ppm]=2.33 (s, 3H), 3.83 (dd, 1H), 3.97 (dd, 1H), 4.21-4.33 (m, 1H), 4.62 (s, 2H), 6.58 (d, 1H), 6.91 (d, 1H), 7.60-7.70 (m, 3H), 7.73-7.81 (m, 4H), 7.91 (s, 1H), 9.63 (d, 1H).

### Example 17

### 2-{3-(4-Chlorophenyl)-5-oxo-4-[(2S)-3,3,3-trifluoro-2-hydroxypropyl]-4,5-dihydro-1H-1,2,4-triazol-1-yl}-N-{(6-methoxypyridin-2-yl)[3-(trifluoromethyl)phenyl]methyl}acetamide (Diastereomer mixture)

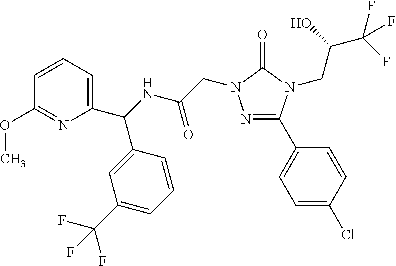

A quantity of 100 mg (0.27 mmol) of the compound from Example 8A was stirred together with 51.7 mg (0.38 mmol) of HOBt, 73.4 mg (0.38 mmol) of EDC, 113 mg of the compound from Example 29A (about 0.30 mmol) and 57 μl (0.33 mmol) of N,N-diisopropylethylamine in 3.3 ml of DMF at RT for 1 hour. The reaction mixture was then separated by preparative HPLC (Method 10). This gave 155 mg (90% of theory) of the title compound as a diastereomer mixture.

LC-MS [Method 5]: Rₜ=1.26 min; MS [ESpos]: m/z=630 (M+H)⁺

¹H-NMR (400 MHz, DMSO-d₆): δ [ppm]=3.79-3.87 (m, 4H), 3.93-4.01 (m, 1H), 4.21-4.33 (m, 1H), 4.58-4.70 (m, 2H), 6.17 (d, 1H), 6.72 (d, 1H), 6.90 (d, 1H), 7.12 (d, 1H), 7.54-7.65 (m, 4H), 7.67-7.77 (m, 4H), 7.84 (s, 1H), 9.09 (d, 1H).

A quantity of 50 mg of the diastereomer mixture obtained was resolved by chromatography on a chiral phase (Method 11b) into the individual diastereomers: see Example 18 and Example 19.

### Example 18

### 2-{3-(4-Chlorophenyl)-5-oxo-4-[(2S)-3,3,3-trifluoro-2-hydroxypropyl]-4,5-dihydro-1H-1,2,4-triazol-1-yl}-N-{(6-methoxypyridin-2-yl)[3-(trifluoromethyl)phenyl]methyl}acetamide (Diastereomer 1)

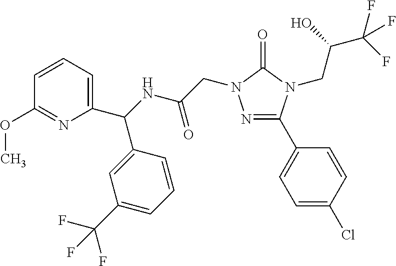

First-eluting diastereomer from the chromatographic diastereomer separation by Method 11b of 50 mg of the compound from Example 17. The product obtained was further purified by preparative HPLC (Method 10). This gave 24 mg of the title compound.

Analytical chiral HPLC (Method 12b): Rₜ=11.43 min

LC-MS [Method 4]: Rₜ=1.25 min; MS [ESpos]: m/z=630 (M+H)⁺

¹H-NMR (400 MHz, DMSO-d₆): δ [ppm]=3.82 (s, 3H), 3.83 (dd, 1H), 3.97 (dd, 1H), 4.21-4.32 (m, 1H), 4.58-4.70 (m, 2H), 6.17 (d, 1H), 6.72 (d, 1H), 6.91 (d, 1H), 7.12 (d, 1H), 7.56 (t, 1H), 7.60-7.65 (m, 3H), 7.67-7.77 (m, 4H), 7.84 (s, 1H), 9.09 (d, 1H).

### Example 19

### 2-{3-(4-Chlorophenyl)-5-oxo-4-[(2S)-3,3,3-trifluoro-2-hydroxypropyl]-4,5-dihydro-1H-1,2,4-triazol-1-yl}-N-{(6-methoxypyridin-2-yl)[3-(trifluoromethyl)phenyl]methyl}acetamide (Diastereomer 2)

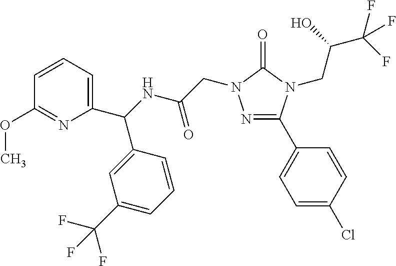

Last-eluting diastereomer from the chromatographic diastereomer separation by Method 11b of 50 mg of the compound from Example 17. The product obtained was further purified by preparative HPLC (Method 10). This gave 23 mg of the title compound.

Analytical chiral HPLC (Method 12b): Rₜ=20.09 min

LC-MS [Method 4]: Rₜ=1.24 min; MS [ESpos]: m/z=630 (M+H)⁺

¹H-NMR (400 MHz, DMSO-d₆): δ [ppm]=3.82 (s, 3H), 3.83 (dd, 4H), 3.97 (dd, 1H), 4.21-4.33 (m, 1H), 4.64 (s, 2H), 6.17 (d, 1H), 6.72 (d, 1H), 6.90 (d, 1H), 7.12 (d, 1H), 7.57 (t, 1H), 7.60-7.65 (m, 3H), 7.67-7.77 (m, 4H), 7.84 (s, 1H), 9.09 (d, 1H).

### Example 20

### 2-{3-(4-Chlorophenyl)-5-oxo-4-[(2S)-3,3,3-trifluoro-2-hydroxypropyl]-4,5-dihydro-1H-1,2,4-triazol-1-yl}-N-{(6-hydroxypyridin-2-yl)[3-(trifluoromethyl)phenyl]methyl}acetamide (Diastereomer mixture)

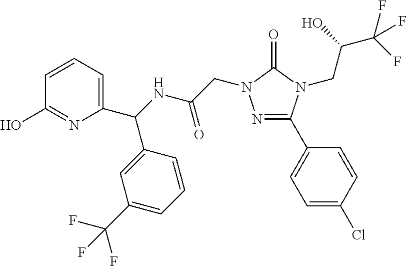

In a pressure vessel, 19 μl (0.15 mmol) of chlorotrimethylsilane and 10.5 mg (0.14 mmol) of sodium sulphide were stirred in 1 ml of dichloromethane at RT for 30 minutes. Then 50 mg (79 μmol) of the compound from Example 17 were added, the vessel was sealed in such a way as to be airtight, and the vessel was heated to 60° C. in a heating bath overnight. Since no reaction occurred, the dichloromethane was removed on a rotary evaporator, the residue was admixed with 2 ml of a 4N solution of hydrogen chloride in dioxane, the vessel was airtightly sealed again, and the mixture was heated to 60° C. overnight. The volatile components were removed on a rotary evaporator and the residue was separated by preparative HPLC (Method 10). This gave 4 mg (8% of theory) of the title compound.

LC-MS [Method 4]: Rₜ=1.03 min; MS [ESpos]: m/z=616 (M+H)⁺

¹H-NMR (400 MHz, DMSO-d₆): δ [ppm]=3.83 (dd, 1H), 3.97 (dd, 1H), 4.21-4.34 (m, 1H), 4.54-4.67 (m, 2H), 5.97 (br d, 1H), 6.16 (br s, 1H), 6.23 (br s, 1H), 6.90 (“dd” (1 d per diastereomer), 1H), 7.42 (br. s., 1H), 7.59-7.78 (m, 8H), 9.15 (d, 1H), 11.73 (br. s., 1H).

### Example 21

### 2-{3-(4-Chlorophenyl)-5-oxo-4-[(2S)-3,3,3-trifluoro-2-hydroxypropyl]-4,5-dihydro-1H-1,2,4-triazol-1-yl}-N-{1H-imidazol-4-yl[3-(trifluoromethyl)phenyl]methyl}acetamide (Diastereomer mixture)

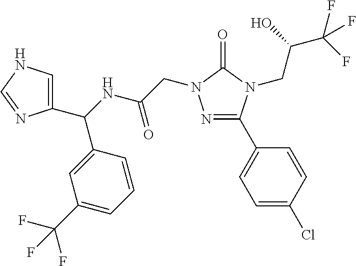

A quantity of 31 mg (84 mmol) of the compound from Example 8A was stirred together with 16 mg (0.12 mmol) of HOBt, 22.5 mg (0.12 mmol) of EDC, 29 mg (92 mmol) of the compound from Example 31A and 32 μl (0.19 mmol) of N,N-diisopropylethylamine in 1 ml DMF at RT for 1 hour. The entire reaction mixture was then separated by preparative HPLC (Method 10). This gave 36 mg (73% of theory) of the title compound as a diastereomer mixture.

LC-MS [Method 5]: Rₜ=0.92 min; MS [ESpos]: m/z=589 (M+H)⁺

¹H-NMR (400 MHz, DMSO-d₆): δ [ppm]=3.82 (dd, 1H), 3.96 (br d, 1H), 4.22-4.34 (m, 1H), 4.57 (s, 2H), 6.10 (d, 1H), 6.92 (“dd”, 1H (1 d per diastereomer)), 7.00 (br. s., 1H), 7.51-7.76 (m, 9H), 9.03 (br. t., 1H), 12.01 (br. s., 1H).

### Example 22

### 2-{3-(4-Chlorophenyl)-5-oxo-4-[(2S)-3,3,3-trifluoro-2-hydroxypropyl]-4,5-dihydro-1H-1,2,4-triazol-1-yl}-N-[1-(2-fluorophenyl)-2-(3-methyl-1,2,4-oxadiazol-5-yl)ethyl]acetamide (Diastereomer mixture)

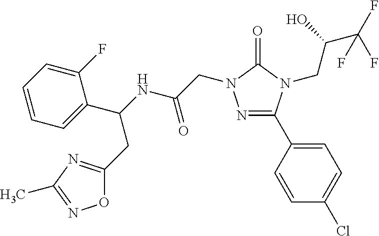

A quantity of 50 mg (0.08 mmol) of the compound from Example 33A was dissolved in 2 ml of toluene, admixed with 13 mg (0.17 mmol) of N-hydroxyacetamidine and 24 mg (0.17 mmol) of potassium carbonate and heated under reflux for 2 hours. For work-up, 10 ml of water were added and the mixture was extracted with three times 10 ml of ethyl acetate. The combined organic phases were dried over magnesium sulphate, filtered and concentrated on a rotary evaporator. The crude product was purified by chromatography on silica gel (eluent: cyclohexane/ethyl acetate 1:1→1:2). This gave 40 mg (85% of theory) of the target compound.

LC-MS [Method 4] Rₜ=1.05 min; MS [ESIpos]: m/z=569 (M+H)⁺

¹H-NMR (400 MHz, CDCl₃): δ [ppm]=□□ 2.28 and 2.34 (2s, 3H), 3.34-3.51 (m, 2H), 3.94-4.16 (m, 2H), 4.37-4.83 (m, 3H), 5.62-5.71 (m, 1H), 5.72 and 5.84 (2d, 1H), 6.98-7.16 (m, 3H), 7.18-7.34 (m, 2H), 7.47-7.56 (m, 2H), 7.67-7.75 (m, 2H). (partial resolution of the doubled signal set of the diastereomer mixture.)

### Example 23

### 2-[3-(4-Chlorophenyl)-4-cyclopropyl-5-oxo-4,5-dihydro-1H-1,2,4-triazol-1-yl]-N-{(6-methoxypyridin-2-yl)[3-(trifluoromethyl)phenyl]methyl}acetamide (Racemate)

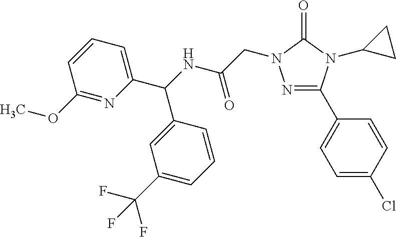

A quantity of 46 mg (157 mmol) of [3-(4-chlorophenyl)-4-cyclopropyl-5-oxo-4,5-dihydro-1H-1,2,4-triazol-1-yl]acetic acid (preparation: see Example 88A in WO 2007/134862) were stirred together with 25 mg (0.19 mmol) of HOBt, 36 mg (0.19 mmol) of EDC, 55 mg (173 mmol) of the compound from Example 29A and 33 μl (0.19 mmol) of N,N-diisopropylethylamine in 1.8 ml of DMF at RT for 1 hour. Then the entire reaction mixture was separated by preparative HPLC (Method 14). The appropriate fraction was freed from the solvents on a rotary evaporator and the residue was dried under a high vacuum. This gave 75 mg (88% of theory) of the title compound.

LC-MS [Method 4]: Rₜ=1.24 min; MS [ESpos]: m/z=558 (M+H)⁺

¹H-NMR (400 MHz, DMSO-d₆): δ [ppm]=0.57 (m, 2H), 0.90 (m, 2H), 3.17 (m, 1H), 3.81 (s, 3H), 4.52-4.62 (m [AB], 2H), 6.16 (d, 1H), 6.71 (d, 1H), 7.11 (d, 1H), 7.53-7.64 (m, 4H), 7.67-7.74 (m, 2H), 7.77-7.84 (m, 3H), 9.04 (d, 1H).

### Example 24

### 2-{3-(4-Chlorophenyl)-5-oxo-4-[(1E)-3,3,3-trifluoroprop-1-en-1-yl]-4,5-dihydro-1H-1,2,4-triazol-1-yl}-N-{(6-methoxypyridin-2-yl)[3-(trifluoromethyl)phenyl]methyl}acetamide (Racemate)

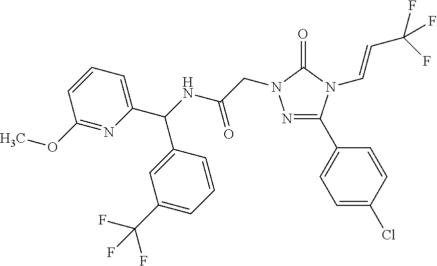

A quantity of 54 mg (157 mmol) of the compound from Example 35A was stirred together with 25 mg (0.19 mmol) of HOBt, 36 mg (0.19 mmol) of EDC, 55 mg (173 mmol) of the compound from Example 29A and 33 μl (0.19 mmol) of N,N-diisopropylethylamine in 1.8 ml of DMF at RT for 1 hour. Then the entire reaction mixture was separated by preparative HPLC (Method 14). The appropriate fraction was freed from the solvents in a rotary evaporator and the residue was dried under a high vacuum. This gave 85 mg (89% of theory) of the title compound.

LC-MS [Method 4]: Rₜ=1.35 min; MS [ESpos]: m/z=612 (M+H)⁺

¹H-NMR (400 MHz, DMSO-d₆): δ [ppm]=3.82 (s, 3H), 4.64-4.74 (m [AB], 2H), 6.19 (d, 1H), 6.72 (d, 1H), 6.85 (dq, J=14.2, 7.1 Hz, 1H), 7.10 (d, 1H), 7.18 (dq, J=14.2, 2.2 Hz, 1H), 7.54-7.75 (m, 8H), 7.84 (br. s, 1H), 9.13 (d, 1H).

### Example 25

### N-{(5-Amino-1,3,4-oxadiazol-2-yl)[3-(trifluoromethyl)phenyl]methyl}-2-{3-(4-chlorophenyl)-5-oxo-4-[(1E)-3,3,3-trifluoroprop-1-en-1-yl]-4,5-dihydro-1H-1,2,4-triazol-1-yl}acetamide (Racemate)

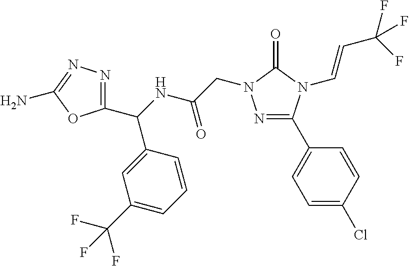

A quantity of 45 mg (130 mmol) of the compound from Example 35A was stirred together with 21 mg (0.16 mmol) of HOBt, 30 mg (0.16 mmol) of EDC, 47 mg (142 mmol) of the compound from Example 25A and 27 μl (0.16 mmol) of N,N-diisopropylethylamine in 1.5 ml of DMF at RT for 1 hour. Then the entire reaction mixture was separated by preparative HPLC (Method 14). The appropriate fraction was freed from the solvents on a rotary evaporator and the residue was dried under a high vacuum. This gave 63 mg (83% of theory) of the title compound.

LC-MS [Method 4]: Rₜ=1.11 min; MS [ESpos]: m/z=588 (M+H)⁺

¹H-NMR (400 MHz, DMSO-d₆): δ [ppm]=4.55-4.70 (m [AB], 2H), 6.38 (d, 1H), 6.87 (dq, J=14.2, 7.0 Hz, 1H), 7.12 (br. s, 2H), 7.18 (dq, J=14.2, 2.2 Hz, 1H), 7.60-7.70 (m, 5H), 7.73 (br t, 2H), 7.84 (br. s, 1H), 9.50 (d, 1H).

### Example 26

### 2-[3-(4-Chlorophenyl)-5-oxo-4-(3,3,3-trifluoropropyl)-4,5-dihydro-1H-1,2,4-triazol-1-yl]-N-{(3-methyl-1,2,4-oxadiazol-5-yl)[3-(trifluoromethyl)phenyl]methyl}acetamide (Racemate)

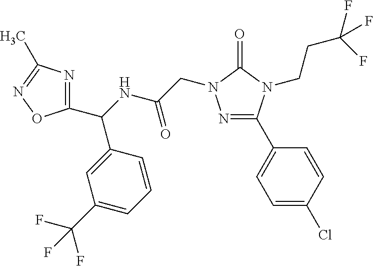

A quantity of 54 mg (155 mmol) of the compound from Example 36A was stirred together with 25 mg (0.19 mmol) of HOBt, 36 mg (0.19 mmol) of EDC, 50 mg (170 mmol) of the compound from Example 27A and 32 μl (0.19 mmol) of N,N-diisopropylethylamine in 1.8 ml of DMF at RT overnight. Then, the entire reaction mixture was separated by preparative HPLC (Method 14). The appropriate fraction was freed from the solvents in a rotary evaporator and the residue was dried under a high vacuum. This gave 71 mg (76% of theory) of the title compound.

LC-MS [Method 2]: Rₜ=2.50 min; MS [ESpos]: m/z=589 (M+H)⁺

¹H-NMR (400 MHz, DMSO-d₆): δ [ppm]=2.33 (s, 3H), 2.53-2.69 (m, 2H), 3.99 (t, 2H), 4.55-4.66 (m [AB], 2H), 6.58 (d., 1H), 7.60-7.69 (m, 5H), 7.77 (br t, 2H), 7.90 (br. s, 1H), 9.61 (d, 1H).

### B. EVALUATION OF THE PHARMACOLOGICAL ACTIVITY

### Abbreviations

EDTA Ethylenediaminetetraacetic acid

### DMEM Dulbecco's Modified Eagle Medium

FCS Foetal calf serum
HEPES 4-(2-Hydroxyethyl)-1-piperazineethanesulphonic acid

### SmGM Smooth Muscle Cell Growth Media

Tris-HCl 2-Amino-2-(hydroxymethyl)-1,3-propanediol hydrochloride

### UtSMC Uterine Smooth Muscle Cells

### B-1. Cellular In Vitro Assay for Determining the Vasopressin Receptor Activity

The identification of agonists and antagonists of the V1a and V2 vasopressin receptors from humans and rats and also the quantification of the activity of the substances described here took place using recombinant cell lines. These cells derive originally from a hamster ovary epithelial cell (Chinese Hamster Ovary, CHO K1, ATCC: American Type Culture Collection, Manassas, Va. 20108, USA). The test cell lines constitutively express a modified form of the calcium-sensitive photoprotein aequorin, which, after reconstitution with the cofactor coelenterazine, emits light when there are increases in the free calcium concentration (Rizzuto R., Simpson A. W., Brini M., Pozzan T.; Nature 358 (1992) 325-327). In addition, the cells are stably transfected with the human or rat V1a or V2 receptors. In the case of the Gs-coupling V2 receptors, the cells are stably transfected with a further gene, which codes for the promiscuous G_{α16} protein (Amatruda T. T., Steele D. A., Slepak V. Z., Simon M. I., Proc. Nat. Acad. Sci. USA 88 (1991), 5587-5591), either independently or as a fusion gene. The resulting vasopressin receptor test cells react to stimulation of the recombinantly expressed vasopressin receptors by intracellular release of calcium ions, which can be quantified by the resulting aequorin luminescence using a suitable luminometer (Milligan G., Marshall F., Rees S., Trends in Pharmaco. Sci. 17 (1996) 235-237).

Test procedure: On the day before the assay, the cells are plated out in culture medium (DMEM, 10% FCS, 2 mM glutamine, 10 mM HEPES) in 384-well microtiter plates and kept in a cell incubator (96% humidity, 5% v/v carbon dioxide, 37° C.). On the day of the assay, the culture medium is replaced by a Tyrode solution (140 mM sodium chloride, 5 mM potassium chloride, 1 mM magnesium chloride, 2 mM calcium chloride, 20 mM glucose, 20 mM HEPES), which additionally contains the cofactor coelenterazine (50 μM), and the microtiter plate is then incubated for a further 3-4 hours. The test substances in various concentrations are placed for 10 to 20 minutes in the wells of the microtiter plate before the agonist [Arg8]-vasopressin is added, and the resulting light signal is measured immediately in the luminometer. The IC50 values are calculated using the GraphPad PRISM computer program (Version 3.02).

The table below lists representative IC₅₀ values for the compounds of the invention on the cell line transfected with the human V1a or V2 receptor:

TABLE 1

| Example No. | IC₅₀ hV1a [μM] | IC₅₀ hV2 [μM] |
|---|---|---|
| 3 | 0.0065 | 0.0032 |
| 4 | 0.0083 | 0.0033 |
| 18 | 0.032 | 0.091 |
| 19 | 0.0046 | 0.0071 |
| 28 | 0.013 | 0.014 |

### B-2. Cellular In Vitro Assay for Detecting the Action of Vasopressin V1a Receptor Antagonists on the Regulation of Pro-Fibrotic Genes

The cell line H9C2 described as of cardiomyocyte type (American Type Culture Collection ATCC No. CRL-1446), isolated from rat cardiac tissue, endogenously expresses the vasopressin V1A receptor AVPR1A in high copy number, whereas the AVPR2 expression cannot be detected. For cell assays on the inhibition of the AVPR1A receptor-dependent regulation of gene expression by receptor antagonists, the procedure is as follows:

H9C2 cells are seeded in 12-well microtiter plates for cell culture, at a cell density of 100 000 cells/well, in 1.0 ml of Opti-MEM medium (Invitrogen Corp. Carlsbad Calif., USA, Cat. No. 11058-021) with 2% FCS and 1% penicillin/streptomycin solution (Invitrogen Cat. No. 10378-016), and held in a cell incubator (96% humidity, 5% v/v carbon dioxide, 37° C.). After 24 hours, sets of three wells (triplicate) are charged with vehicle solution (negative control), vasopressin solution: [Arg8]-vasopressin acetate (Sigma Cat. No. V9879) or test substances (dissolved in vehicle: water with 20% by volume ethanol) and vasopressin solution. In the cell culture, the final vasopressin concentration is 0.05 μM. The test substance solution is added to the cell culture in small volumes, and so a final concentration of 0.1% of ethanol in the cell assay is not exceeded. After an incubation time of 6 hours, the culture supernatant is drawn off under suction, the adherent cells are lysed in 250 μl of RLT buffer (Qiagen, Ratingen, Cat. No. 79216), and the RNA is isolated from this lysate using the RNeasy kit (Qiagen, Cat. No. 74104). This is followed by DNAse digestion (Invitrogen Cat. No. 18068-015), cDNA synthesis (Promaga ImProm-II Reverse Transcription System Cat. No. A3800) and RTPCR using the pPCR MasterMix RT-QP2X-03-075 from Eurogentec, Seraing, Belgium. All procedures take place in accordance with the working protocols of the test reagents' manufacturers. The primer sets for the RTPCR are selected on the basis of the mRNA gene sequences (NCBI Genbank Entrez Nucleotide Data Base) using the Primer3Plus program with 6-FAM-TAMRA labelled probes. The RTPCR for determining the relative mRNA expression in the cells of the various assay batches is carried out using the Applied Biosystems ABI Prism 7700 Sequence Detector in 96-well or 384-well microtiter plate format in accordance with the instrument operating instructions. The relative gene expression is represented by the delta-delta Ct value [Applied Biosystems, User Bulletin No. 2 ABI Prism 7700 SDS Dec. 11, 1997 (updated 10/2001)] with reference to the level of expression of the ribosomal protein L-32 gene (Genbank Acc. No. NM_{—}013226) and the threshold Ct value of Ct=35.

### B-3. In Vivo Test for Detection of Cardiovascular Effect: Blood Pressure Measurement on Anaesthetised Rats (Vasopressin ‘Challenge’ Model)

In male Sprague-Dawley rats (250-350 g body weight) under ketamine/xylazine/pentobarbital injection anaesthesia, polyethylene tubes (PE-50; Intramedic®), which are prefilled with heparin-containing (500 IU/ml) isotonic sodium chloride solution, are introduced into the jugular vein and the femoral vein and then tied in. Via one venous access, with the aid of a syringe, arginine-vasopressin is injected; the test substances are administered via the second venous access. For determination of the cystolic blood pressure, a pressure catheter (Millar SPR-320 2F) is tied into the carotid artery. The arterial catheter is connected to a pressure transducer which feeds its signals to a recording computer equipped with suitable recording software. In a typical experiment the experimental animal is administered 3-4 successive bolus injections at intervals of 10-15 min with a defined amount of arginine-vasopressin (30 ng/kg) in isotonic sodium chloride solution and, when the blood pressure has reached initial levels again, the substance under test is administered as a bolus, with subsequent ongoing infusion, in a suitable solvent. After this, at defined intervals (10-15 min), the same amount of vasopressin as at the start is administered again. On the basis of the blood pressure values, a determination is made of the extent to which the test substance counteracts the hypertensive effect of the vasopressin. Control animals receive only solvent instead of the test substance.

Following intravenous administration, the compounds of the invention, in comparison to the solvent controls, bring about an inhibition in the blood pressure increase caused by arginine-vasopressin.

### B-4. In Vivo Assay for Detecting the Cardiovascular Effect: Diuresis Investigations on Conscious Rats in Metabolism Cages

Wistar rats (220-400 g body weight) are kept with free access to feed (Altromin) and drinking water. During the experiment, the animals are kept with free access to drinking water for 4 to 8 hours individually in metabolism cages suitable for rats of this weight class (Tecniplast Deutschland GmbH, D-82383 Hohenpeiβenberg). At the beginning of the experiment, the animals are administered the substance under test in a volume of 1 to 3 ml/kg body weight of a suitable solvent by means of gavage into the stomach. Control animals receive only solvent. Controls and substance tests are carried out in parallel on the same day. Control groups and substance-dose groups each consist of 4 to 8 animals. During the experiment, the urine excreted by the animals is collected continuously in a receiver at the base of the cage. The volume of urine per unit time is determined separately for each animal, and the concentration of the sodium and potassium ions excreted in the urine is measured by standard methods of flame photometry. To obtain a sufficient volume of urine, the animals are given a defined amount of water by gavage at the beginning of the experiment (typically 10 ml per kilogram of body weight). Before the beginning of the experiment and after the end of the experiment, the body weight of the individual animals is taken.

Following oral administration, in comparison with control animals, the compounds of the invention bring about an increased excretion of urine, which is based essentially on an increased excretion of water (aquaresis).

### B-5. In Vivo Assay for Detecting the Cardiovascular Effect: Haemodynamic Investigations on Anaesthetised Dogs

Male or female mongrel dogs (Mongrels, Marshall BioResources, USA) with a weight of between 20 and 30 kg are anaesthetised with pentobarbital (30 mg/kg iv, Narcoren®, Merial, Germany) for the surgical interventions and the haemodynamic and functional investigation terminii. Alcuronium chloride (Alloferin®, ICN Pharmaceuticals, Germany, 3 mg/animal iv) serves additionally as a muscle relaxant. The dogs are intubated and ventilated with an oxygen/ambient air mixture (40/60%) (about 5-6 L/min) Ventilation takes place using a ventilator from Draeger (Sulla 808) and is monitored using a carbon dioxide analyser (Engström).

The anaesthesia is maintained by continual infusion of pentobarbital (50 μg/kg/min); fentanyl is used as an analgesic (10 μg/kg/h). One alternative to pentobarbital is to use isoflurane (1-2% by volume).

In preparatory interventions, the dogs are fitted with a cardiac pacemaker.
At a time of 21 days before the first drug testing (i.e. start of experiment), a cardiac pacemaker from Biotronik (Logos®) is implanted into a subcutaneous skin pocket and is contacted with the heart via a pacemaker electrode which is advanced through the external jugular vein, with illumination, into the right ventricle.
At the same time as the implanting of the pacemaker, through retrograde advancing of a 7F biopsy forceps (Cordis) via a sheath introducer (Avanti+®; Cordis) in the fermoral artery, and after atraumatic passage through the aortic valve, there is defined lesion of the mitral valve, with monitoring by echo cardiography and illumination. Thereafter all of the accesses are removed and the dog wakes spontaneously from the anaesthesia.
After a further 7 days (i.e. 14 days before the first drug testing), the above pacemaker is activated and the heart is stimulated at a frequency of 220 beats per minute.

The actual drug testing experiments take place 14 and 28 days after the beginning of pacemaker stimulation, using the following instrumentation:
Bladder catheter for bladder relief and for measuring the flow of urine
ECG leads to the extremities (for ECG measurement)
Introduction of an NaCl-filled Fluidmedic PE-300 tube into the femoral artery. This tube is connected to a pressure sensor (Braun Melsungen, Melsungen, Germany) for measuring the systemic blood pressure
Introduction of a Millar Tip catheter (type 350 PC, Millar Instruments, Houston, USA) through the left atrium or through a port secured in the carotid artery, for measuring cardiac haemodynamics
Introduction of a Swan-Ganz catheter (CCOmbo 7.5F, Edwards, Irvine, USA) via the jugular vein into the pulmonary artery, for measuring the cardiac output, oxygen saturation, pulmonary arterial pressures and central venous pressure
Siting of a Braunüle in the cephalic vein, for infusing pentobarbital, for liquid replacement and for blood sampling (determination of the plasma levels of substance or other clinical blood values)
Siting of a Braunüle in the saphenous vein, for infusing fentanyl and for administration of substance
Infusion of vasopressin (Sigma) in increasing dosage, up to a dose of 4 mU/kg/min. The pharmacological substances are then tested with this dosage.

The primary signals are amplified if necessary (Gould amplifier, Gould Instrument Systems, Valley View, USA) or Edwards Vigilance-Monitor (Edwards, Irvine, USA) and subsequently fed into the Ponemah system (DataSciences Inc, Minneapolis, USA) for evaluation. The signals are recorded continuously throughout the experimental period, and are further processed digitally by the said software, and averaged over 30 s.

### C. EXEMPLARY EMBODIMENTS OF PHARMACEUTICAL COMPOSITIONS

The compounds of the invention can be converted into pharmaceutical preparations in the following ways:

### Tablet:

### Composition:

100 mg of the compound of the invention, 50 mg of lactose (monohydrate), 50 mg of corn starch (native), 10 mg of polyvinylpyrrolidone (PVP 25) (BASF, Ludwigshafen, Germany) and 2 mg of magnesium stearate.

Tablet weight 212 mg. Diameter 8 mm, radius of curvature 12 mm

### Production:

The mixture of compound of the invention, lactose and starch is granulated with a 5% strength solution (m/m) of the PVP in water. After drying, the granules are mixed with the magnesium stearate for 5 minutes. This mixture is compressed using a conventional tableting press (for tablet format see above). The guideline compressive force used for compression is 15 kN.

### Suspension for Oral Administration:

### Composition:

1000 mg of the compound of the invention, 1000 mg of ethanol (96%), 400 mg of Rhodigel® (xanthan gum from FMC, Pennsylvania, USA) and 99 g of water.

A single dose of 100 mg of the compound of the invention is given by 10 ml of oral suspension.

### Production:

The Rhodigel is suspended in ethanol, and the compound of the invention is added to the suspension. The water is added with stirring. Stirring is continued for about 6 h until the swelling of the Rhodigel is ended.

### Solution for Oral Administration:

### Composition:

500 mg of the compound of the invention, 2.5 g of polysorbate and 97 g of polyethylene glycol 400. A single dose of 100 mg of the compound of the invention is given by 20 g of oral solution.

### Production:

The compound of the invention is suspended with stiffing in the mixture of polyethylene glycol and polysorbate. The stirring operation continues until the compound of the invention is fully dissolved.

### I.V. Solution:

The compound of the invention is dissolved at a concentration below saturation solubility in a physiologically tolerated solvent (e.g. isotonic saline solution, 5% glucose solution and/or 30% PEG 400 solution). The solution is sterile-filtered and dispensed into sterile, pyrogen-free injection containers.

1. A compound of the formula (I) 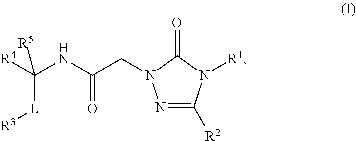
in which
L is a bond or —C(R^{6A}R^{6B})—*,
where
* is the attachment site to R³,
R^{6A} is hydrogen, (C₁-C₄) alkyl or trifluoromethyl,
R^{6B} is hydrogen or (C₁-C₄) alkyl,
R¹ is (C₁-C₆) alkyl, (C₂-C₆) alkenyl, (C₂-C₆) alkynyl or (C₃-C₇) cycloalkyl,
where (C₁-C₆) alkyl, (C₂-C₆) alkenyl and (C₂-C₆) alkynyl may be substituted by 1 to 3 substituents independently of one another selected from the group consisting of halogen, cyano, oxo, hydroxyl, trifluoromethyl, (C₃-C₇) cycloalkyl, (C₁-C₆) alkoxy, trifluoromethoxy and phenyl,
in which (C₃-C₇) cycloalkyl may be substituted by 1 or 2 substituents independently of one another selected from the group consisting of halogen, (C₁-C₄) alkyl, oxo, hydroxyl, (C₁-C₄) alkoxy and amino,
and
in which (C₁-C₆) alkoxy may be substituted by 1 or 2 substituents independently of one another selected from the group consisting of amino, hydroxyl, (C₁-C₄) alkoxy, hydroxycarbonyl and (C₁-C₄) alkoxycarbonyl,
and
in which phenyl may be substituted by 1 to 3 substituents independently of one another selected from the group consisting of halogen, cyano, nitro, (C₁-C₄) alkyl, trifluoromethyl, hydroxyl, hydroxymethyl, (C₁-C₄) alkoxy, trifluoromethoxy, (C₁-C₄) alkoxymethyl, hydroxycarbonyl, (C₁-C₄) alkoxycarbonyl, aminocarbonyl, mono-(C₁-C₄) alkylaminocarbonyl and di-(C₁-C₄) alkylaminocarbonyl,
and
where (C₃-C₇) cycloalkyl may be substituted by 1 or 2 substituents independently of one another selected from the group consisting of fluorine, (C₁-C₄) alkyl, (C₁-C₄) alkoxy, hydroxy, amino and oxo,
R² is phenyl, thienyl or furyl,
where phenyl, thienyl and furyl may be substituted by 1 to 3 substituents independently of one another selected from the group consisting of halogen, cyano, nitro, (C₁-C₄) alkyl, trifluoromethyl, hydroxyl, (C₁-C₄) alkoxy and trifluoromethoxy,
R³ is a 5- or 6-membered heterocyclyl or 5- or 6-membered heteroaryl,
where 5- or 6-membered heterocyclyl may be substituted by 1 to 3 substituents independently of one another selected from the group consisting of halogen, trifluoromethyl, (C₁-C₄) alkyl, hydroxyl, oxo, trifluoromethoxy, (C₁-C₄) alkoxy, amino, mono-(C₁-C₄)-alkylamino, di-(C₁-C₄)-alkylamino, (C₁-C₄) alkylthio and thiooxo,
where 5- or 6-membered heteroaryl may be substituted by 1 to 3 substituents independently of one another selected from the group consisting of halogen,
trifluoromethyl, (C₁-C₄) alkyl, hydroxyl, trifluoromethoxy, (C₁-C₄) alkoxy, amino, mono-(C₁-C₄)-alkylamino, di-(C₁-C₄)-alkylamino and (C₁-C₄) alkylthio,
R⁴ is phenyl, naphthyl or 5- to 10-membered heteroaryl,
where phenyl, naphthyl and 5- to 10-membered heteroaryl may be substituted by 1 to 3 substituents independently of one another selected from the group consisting of halogen, cyano, nitro, (C₁-C₄) alkyl, difluoromethyl, trifluoro-methyl, hydroxyl, (C₁-C₄) alkoxy, difluoromethoxy and trifluoromethoxy,
R⁵ is hydrogen, trifluoromethyl or (C₁-C₄) alkyl,
or a salt thereof.

2. The compound of claim 1, in which
L is a bond or —C(R^{6A}R^{6B})—*,
where
* is the attachment site to R³,
R^{6A} is hydrogen or methyl,
R^{6B} is hydrogen or methyl,
R¹ is (C₁-C₆) alkyl, (C₂-C₆) alkenyl or (C₃-C₆) cycloalkyl,
where (C₁-C₆) alkyl and (C₂-C₆) alkenyl may be substituted by 1 to 3 substituents independently of one another selected from the group consisting of fluorine, chlorine, cyano, oxo, hydroxyl, trifluoromethyl, (C₃-C₆) cycloalkyl, (C₁-C₄) alkoxy, trifluoromethoxy and phenyl,
in which (C₃-C₆) cycloalkyl may be substituted by 1 or 2 substituents independently of one another selected from the group consisting of fluorine, methyl, ethyl, oxo, hydroxyl, methoxy, ethoxy and amino,
and
in which phenyl may be substituted by a substituent selected from the group consisting of fluorine, chlorine, cyano, methyl, ethyl, trifluoromethyl, methoxy, ethoxy, trifluoromethoxy, methoxymethyl, ethoxymethyl, hydroxycarbonyl, methoxycarbonyl, ethoxycarbonyl and aminocarbonyl,
and
where (C₃-C₆) cycloalkyl may be substituted by 1 or 2 substituents independently of one another selected from the group consisting of fluorine, methyl, ethyl, methoxy, ethoxy, hydroxyl, amino and oxo,
R² is phenyl or thienyl,
where phenyl and thienyl may be substituted by 1 or 2 substituents independently of one another selected from the group consisting of fluorine, chlorine, methyl, ethyl, trifluoromethyl, hydroxyl, methoxy, ethoxy and trifluoromethoxy,
R³ is 2-oxo-1,3-oxazolidin-5-yl, 2-oxo-1,3-oxazolidin-4-yl, 2-oxoimidazolidin-4-yl, 2-oxo-2,3-dihydro-1H-imidazol-4-yl, 4,5-dihydro-1H-imidazol-2-yl, 4,5-dihydro-1H-imidazol-4-yl, 4,5-dihydro-1H-imidazol-1-yl, 2-oxo-2,3-dihydro-1,3-oxazol-4-yl, 2-oxo-2,3-dihydro-1,3-oxazol-5-yl, 4,5-dihydro-1,3-oxazol-2-yl, 4,5-dihydro-1,3-oxazol-4-yl, 4,5-dihydro-1,3-oxazol-5-yl, 4,5-dihydro-5-oxo-1H-1,2,4-triazol-3-yl, 4,5-dihydro-5-oxo-1H-1,2,4-oxadiazol-3-yl, 4,5-dihydro-5-oxo-1,3,4-oxadiazol-2-yl, 4,5-dihydro-5-oxo-1H-1,2,4-thiadiazol-3-yl, 2,3-dihydro-2-oxo-1,3,4-thiadiazol-5-yl, furyl, thienyl, thiazolyl, oxazolyl, isothiazolyl, isoxazolyl, pyrazolyl, imidazolyl, triazolyl, oxadiazolyl, thiadiazolyl, tetrazolyl, pyridyl, pyrimidinyl, pyridazinyl, pyrazinyl or triazinyl,
it being possible for 2-oxo-1,3-oxazolidin-5-yl, 2-oxo-1,3-oxazolidin-4-yl, 2-oxo-imidazolidin-4-yl, 2-oxo-2,3-dihydro-1H-imidazol-4-yl, 2-oxo-2,3-dihydro-1,3-oxazol-4-yl, 2-oxo-2,3-dihydro-1,3-oxazol-5-yl, 4,5-dihydro-5-oxo-1H-1,2,4-triazol-3-yl, 4,5-dihydro-5-oxo-1H-1,2,4-oxadiazol-3-yl, 4,5-dihydro-5-oxo-1,3,4-oxadiazol-2-yl, 4,5-dihydro-5-oxo-1H-1,2,4-thiadiazol-3-yl, 2,3-dihydro-2-oxo-1,3,4-thiadiazol-5-yl to be substituted by 1 or 2 substituents independently of one another selected from the group consisting of trifluoromethyl, methyl and ethyl,
and
it being possible for 4,5-dihydro-1H-imidazol-2-yl, 4,5-dihydro-1H-imidazol-4-yl, 4,5-dihydro-1H-imidazol-1-yl, 4,5-dihydro-1,3-oxazol-2-yl, 4,5-dihydro-1,3-oxazol-4-yl, 4,5-dihydro-1,3-oxazol-5-yl to be substituted by 1 or 2 substituents independently of one another selected from the group consisting of oxo, methyl and ethyl,
and
it being possible for furyl, thienyl, thiazolyl, oxazolyl, isothiazolyl, isoxazolyl, pyrazolyl, imidazolyl, triazolyl, oxadiazolyl, thiadiazolyl, tetrazolyl, pyridyl, pyrimidinyl, pyridazinyl, pyrazinyl and triazinyl to be substituted by 1 or 2 substituents independently of one another selected from the group consisting of fluorine, chlorine, trifluoromethyl, methyl, ethyl, hydroxyl, trifluoromethoxy, methoxy, ethoxy, amino, methylamino, ethylamino, dimethylamino, methylethylamino and diethylamino,
R⁴ is phenyl,
where phenyl may be substituted by 1 to 3 substituents independently of one another selected from the group consisting of fluorine, chlorine, cyano, methyl, ethyl, difluoromethyl, trifluoromethyl, hydroxyl, methoxy, ethoxy, difluoromethoxy and trifluoromethoxy,
R⁵ is hydrogen, methyl or ethyl,
or a salt thereof.

3. The compound of claim 1, in which
L is a bond or —C(R^{6A}R^{6B})—*,
where
* is the attachment site to R³,
R^{6A} is hydrogen,
R^{6B} is hydrogen,
R¹ is (C₂-C₄) alkyl, (C₂-C₄) alkenyl or cyclopropyl,
where (C₂-C₄) alkyl and (C₂-C₄) alkenyl are substituted by 1 or 2 substituents independently of one another selected from the group consisting of fluorine, hydroxyl, oxo and trifluoromethyl,
R² is phenyl,
where phenyl is substituted by a substituent selected from the group consisting of fluorine and chlorine,
R³ is a group of the formula 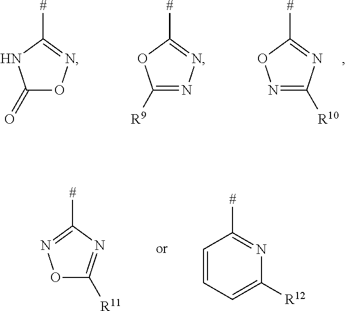
where
# is the attachment site to L,
R⁹ is hydrogen, trifluoromethyl, methyl or amino,
R¹⁰ is trifluoromethyl, methyl or amino,
R¹¹ is hydrogen, fluorine, trifluoromethyl or methyl,
R¹² is hydroxyl or methoxy,
R⁴ is a group of the formula 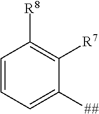
where
## is the attachment site to —C(R⁵)(LR³)N—,
R⁷ is hydrogen, fluorine, chlorine, trifluoromethyl and methoxy,
R⁸ is hydrogen, fluorine, chlorine, trifluoromethyl and methoxy,
where at least one of the radicals R⁷ and R⁸ is other than hydrogen,
R⁵ is hydrogen or methyl,
or a salt, solvate, or a solvate of a salt thereof.

4. A process for preparing a compound of the formula (I) as defined in claim 1, comprising
[A] coupling a compound of the formula (II) 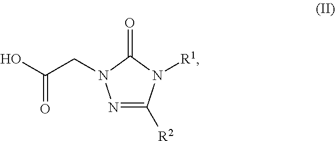
in which R¹ and R² are each as defined in claim 1
in an inert solvent, with activation of the carboxylic acid function, to a compound of the formula (III) 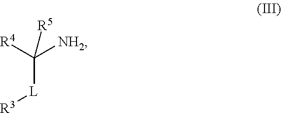
in which L, R³, R⁴ and R⁵ are each as defined in claim 1,
or
[B] reacting a compound of the formula (IV) 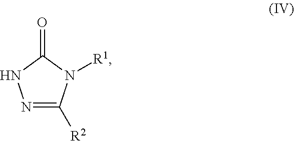
in which R¹ and R² are each as defined in claims 1 to 3
in an inert solvent, in the presence of a base, with a compound of the formula (V) 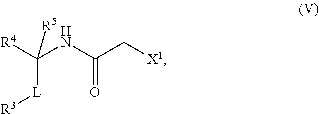
in which L, R³, R⁴ and R⁵ are each as defined in claim 1
and
X¹ is a leaving group, such as halogen, mesylate or tosylate, for example,
or
[C] reacting a compound of the formula (VI) 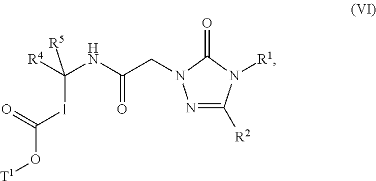
in which L, R¹, R², R⁴ and R⁵ are each as defined in claim 1,
and
T¹ is hydrogen or (C₁-C₄) alkyl,
in an inert solvent, optionally with activation of the carboxylic acid function with hydrazine, to give a compound of the formula (VII) 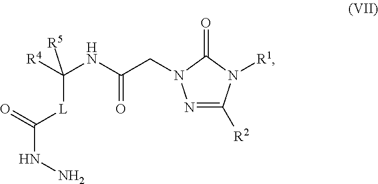
in which L, R¹, R², R⁴ and R⁵ are each as defined in claim 1,
cyclizing the compound of formula (VII) in an inert solvent, optionally in the presence of a suitable base, with cyanogen bromide or a compound of the formula (VIII) 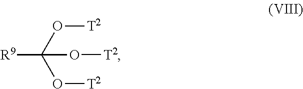
in which
R⁹ is (C₁-C₄) alkyl,
and
T² is (C₁-C₄) alkyl,
to give a compound of the formula (I-C1) or (I-C2) 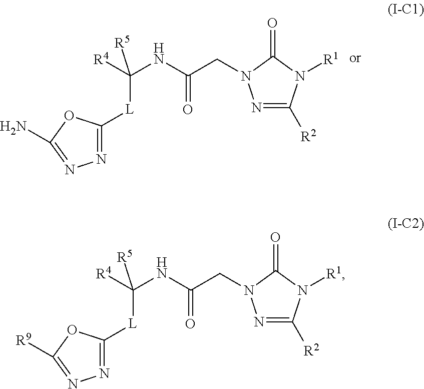
in which L, R¹, R², R⁴, R⁵ and R⁹ are each as defined in claim 1,
or
[D] reacting a compound of the formula (VI) in an inert solvent, optionally with activation of the carboxylic acid function, with a compound of the formula (IX) 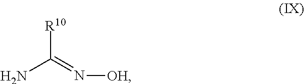
in which R¹⁰ is as defined in claim 1,
and cyclizing the resulting intermediate in a suitable solvent to give a compound of the formula (I-D) 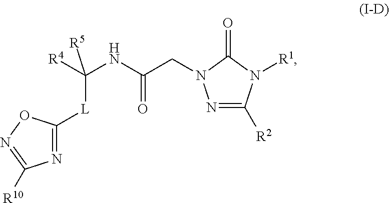
in which L, R¹, R², R⁴, R⁵ and R¹⁰ are each as defined in claim 1,
or
[E] reacting a compound of the formula (X) 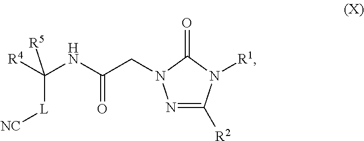
in which L, R¹, R², R⁴ and R⁵ are each as defined in claim 1,
in an inert solvent in the presence of suitable base with hydroxylamine hydrochloride to give a compound of the formula (XI) 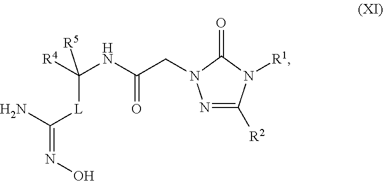
in which L, R¹, R², R⁴ and R⁵ are each as defined in claim 1,
and cyclizing the compound of formula (XI) in an inert solvent with a compound of the formula (XII-1) or (XII-2) 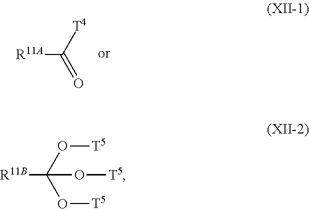
in which
R^{11A} is trifluoromethyl or (C₁-C₄) alkyl,
R^{11B} is hydrogen, trifluoromethyl or (C₁-C₄) alkyl,
T⁴ is chlorine, hydroxyl, (C₁-C₄) alkoxy, trifluoromethylcarbonyloxy or (C₁-C₄) alkylcarbonyloxy,
T⁵ is (C₁-C₄) alkyl,
to give a compound of the formula (I-E1) or (I-E2) 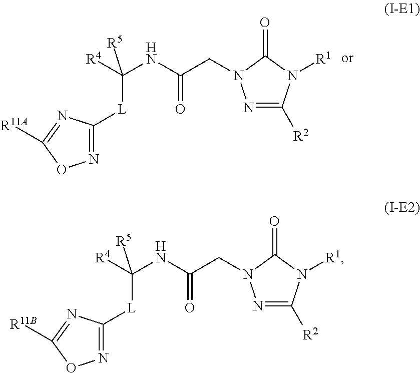
in which L, R¹, R², R⁴, R⁵, R^{11A} and R^{11B} are each as defined in claim 1,
or
[F]cyclizing a compound of the formula (X) 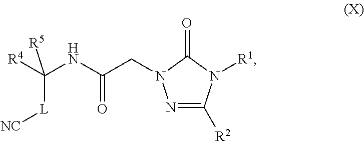
in which L, R¹, R², R⁴ and R⁵ are each as defined in claim 1,
in an inert solvent in the presence of a suitable base with an azide reagent to give a compound of the formula (I-F) 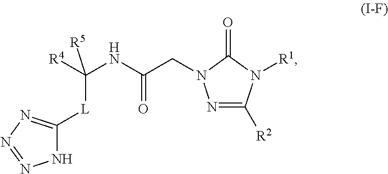
in which L, R¹, R², R⁴ and R⁵ are each as defined in claim 1,
or
[G] reacting a compound of the formula (XI) in an inert solvent in the presence of a suitable base with phosgene, a phosgene derivative such as di- or triphosgene, N,N-carbonyldiimidazole or a chloroformic ester, to produce an intermediate, and
cyclizing the intermediate is in an inert solvent, optionally in the presence of a suitable base, to give a compound of the formula (I-G) 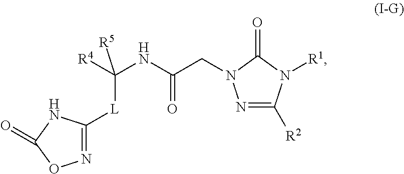
in which L, R¹, R², R⁴ and R⁵ are each as defined in claim 1,
and optionally converting the resulting compound of the formula (I), (I-C1), (I-C2), (I-D), (I-E1), (I-E2), (I-F) or (I-G) with a corresponding (i) solvent and/or (ii) base or acid into a salt thereof.

5. (canceled)

6. (canceled)

7. (canceled)

8. A pharmaceutical composition comprising a compound of claim 1 and an inert, non-toxic, pharmaceutically suitable excipient.

9. The pharmaceutical composition of claim 8, further comprising an active ingredients selected from the group consisting of a diuretic, an angiotensin AII antagonist, an ACE inhibitor, a beta receptor blocker, a mineralocorticoid receptor antagonist, an organic nitrate, an NO donor, and a substance with positive inotropic activity.

10. (canceled)

11. A method for the treatment and/or prophylaxis of acute and chronic cardiac insufficiency, hypervolaemic and euvolaemic hyponatraemia, liver cirrhosis, ascites, oedemas and the syndrome of inadequate ADH secretion (SIADH) in a human or animal, comprising administering an effective amount of at least one compound of claim 1 to the human or animal.

12. The method of claim 11 wherein the compound of claim 1 is administered in the form of the pharmaceutical composition of claim 8.
